(19) United States
(12) Patent Application Publication
(10) Pub. No.: US 2022/0095982 A1
(43) Pub. Date: Mar. 31, 2022

(54) ELECTROCARDIOGRAM PROCESSING SYSTEM FOR DETECTING AND/OR PREDICTING CARDIAC EVENTS

(71) Applicant: Cardiologs Technologies SAS, Paris (FR)

(72) Inventors: Marie-Albane de SAINT VICTOR, Paris (FR); Helene EVAIN, Maisons-Laffitte (FR); Aurelie DELEFORGE, Chaponost (FR); Armand FOUCAULT, Paris (FR); Wadii HAJJI, Vitry sur Seine (FR); Jeremy CALDAS, Asnieres-sur-seine (FR); Benjamin BARRE, Suresnes (FR); Gautier ZIMMERMANN, Paris (FR); Yann FLEUREAU, Paris (FR); Baptiste Rios CAMPO, Paris (FR); Chiara SCABELLONE, Paris (FR); Anastasiya BODROVA, Orsay (FR); Johanna LAVERSIN, Montrouge (FR)

(73) Assignee: Cardiologs Technologies SAS, Paris (FR)

(21) Appl. No.: 17/489,153

(22) Filed: Sep. 29, 2021

Related U.S. Application Data
(60) Provisional application No. 63/226,117, filed on Jul. 27, 2021.
(60) Provisional application No. 63/085,827, filed on Sep. 30, 2020.

(30) Foreign Application Priority Data
Dec. 15, 2020 (EP) 20306567.7

(51) Int. Cl. A61B 5/361; A61B 5/024; A61B 5/08; A61B 5/145; A61B 5/339; A61B 5/00

(52) U.S. Cl. CPC A61B 5/361; A61B 5/02427; A61B 5/0816; A61B 5/14542; A61B 5/6801; A61B 5/7267; A61B 5/7275; A61B 5/6846; A61B 5/339

(57) ABSTRACT

Systems and methods are provided for analyzing electrocardiogram (ECG) data of a patient using a substantial amount of ECG data. The systems receive ECG data from a sensing device positioned on a patient such as one or more ECG leads/electrodes that may be integrated in a smart device. The system may include an application that communicates with an ECG platform running on a server(s) that processes and analyzes the ECG data, e.g., using neural networks, to detect and/or predict various abnormalities, conditions and/or descriptors. The system may also determine a confidence score corresponding to the abnormalities, conditions and/or descriptors. The processed ECG data is used to generate a graphic user interface that is communicated from the server(s) to a computer for display in a user-friendly and interactive manner with enhanced accuracy.

### CROSS-REFERENCE TO RELATED APPLICATIONS

This application claims priority to U.S. Provisional Application Ser. No. 63/226,117, filed Jul. 27, 2021, European Patent Application No. 20306567.7, filed Dec. 15, 2020, and U.S. Provisional Application Ser. No. 63/085,827, filed Sep. 30, 2020, the entire contents of each of which are incorporated herein by reference.

### TECHNICAL FIELD

The present disclosure relates, in general, to an electrocardiogram (ECG) processing system, for example, an ECG system with artificial intelligence and machine learning functionality for detecting and/or predicting cardiac events such as arrhythmias and abnormalities.

### BACKGROUND

An electrocardiogram (ECG) receives electrical cardiac signals from the heart that may be digitized and recorded by a computing device. An ECG typically is generated from cardiac signals sensed by a number of electrodes placed in specific areas on a patient. It is a simple, non-invasive tool, that may be used by most any healthcare professional.

A cardiac signal is composed of one or multiple synchronized temporal signals. FIG. 1A illustrates a recording of a standard 12-lead resting ECG. As is shown in FIG. 1A, each lead generates an electrical signal, resulting in 12 electrical signals. Though the ECG illustrated in FIG. 1A involves 12 leads resulting in 12 recordings, some ECGs may involve fewer leads resulting in fewer recordings. As is shown in FIG. 1A, a cardiac signal displays repeating patterns usually comprising a P-wave, a QRS complex, and a T-wave. As the name suggests, a QRS complex includes a Q-wave, an R-wave and an S-wave. An exemplary P-wave, QRS complex, and T-wave is illustrated in FIG. 1B, which focuses on a couple of beats in one lead signal, showing one R-R interval.

To make a diagnosis, a trained healthcare professional may analyze the ECG recording to identify any abnormalities and/or episodes. It is estimated that about 150 measurable abnormalities may be identified on an ECG recordings today. However, specific expertise and/or training is required to identify abnormalities from an ECG. ECG analysis is only available to those patients that can afford healthcare professions having the appropriate expertise and who otherwise have access to these professionals.

Telecardiology centers have been developed to provide ECG analysis to patients that may not otherwise have access to these trained healthcare professionals. Typically, an ECG recording is generated offsite by a non-specialist and is sent to the telecardiology center for analysis by a cardiologist or by a specialized ECG technician. While the results are generally high quality, the process may be slow and expensive.

Software systems have also been developed as an alternative to analysis by a trained professional. Current software systems provide a low quality interpretation that often results in false positives. Today, these interpretation systems may generate two types of information about a cardiac signal, (1) temporal location information for each wave, referred to as delineation, and (2) global information providing a classification of the cardiac signal or labeling its abnormalities, referred to as classification.

Concerning delineation, two main approaches are used for finding the waves of cardiac signals. The first approach is based on multiscale wavelet analysis. This approach looks for wavelet coefficients reaching predefined thresholds at specified scales. (See Martinez et al., A wavelet-based ECG delineator: evaluation on standard databases, IEEE transactions on biomedical engineering, Vol. 51, No. 4, April 2004, pp. 570-58; Almeida et al., IEEE transactions on biomedical engineering, Vol. 56, No. 8, August 2009, pp 1996-2005; Boichat et al., Proceedings of Wearable and Implantable Body Sensor Networks, 2009, pp. 256-261; U.S. Pat. No. 8,903,479 to Zoicas et al.). The usual process involves identifying QRS complexes, then P-waves, and finally T-waves. This approach is made unstable by the use of thresholds and fails to identify multiple P-waves and “hidden” P-waves.

The second delineation approach is based on Hidden Markov Models (HMM). This machine learning approach treats the current state of the signal as a hidden variable that one wants to recover (Coast et al., IEEE transactions on biomedical engineering, Vol. 37, No. 9, September 1990, pp 826-836; Hughes et al., Proceedings of Neural Information Processing Systems, 2004, pp 611-618; U.S. Pat. No. 8,332,017 to Trassenko et al.). While this approach is an improvement upon on the first delineation approach described above, a representation of the signal must be designed using handcrafted “features,” and a mathematical model must be fitted for each wave, based on these features. Based on a sufficient number of examples, the algorithms may learn to recognize each wave. This process may however be cumbersome and inaccurate due to its dependence on handcrafted features. Specifically, features which have been handcrafted will always be suboptimal since they were not learnt and the process of handcrafting features may have ignored or eliminated crucial information. Further, the model, usually Gaussian, is not well adapted. Also, the current models fail to account for hidden P waves.

Regarding classification, in current systems analysis is only performed on the QRS complex. For example, analysis of a QRS complex may detect ventricular or paced beats. The training involves handcrafted sets of features and corresponding beat labels (Chazal et al., IEEE Transactions on Biomedical Engineering, 2004, vol. 51, pp. 1196- 1206). As explained above, features that have been handcrafted will always be suboptimal since they were not learnt and the process of handcrafting features may have ignored or eliminated crucial information.

To solve the above issues, recent works (Kiranyaz et al., IEEE Transactions on Biomedical Engineering, 2016, Vol. 63, pp 664-675) have turned to novel architectures called neural networks which have been intensively studied and had great results in the field of imaging (Russakovsky et al., arXiv: 1409.0575v3, 30 Jan. 2015). Neural networks learn from raw or mildly preprocessed data and thus bypass the need of handcrafted features. While the application of neural networks is an improvement on the delineation and classification approaches described above, current systems have certain drawbacks. For example, the current neural networks were only developed for QRS characterization. Further, current neural networks processes information in a beat-by-beat manner which fails to capture contextual information from surrounding beats.

Concerning identifying abnormalities and/or cardiovascular disease detection, most algorithms use rules based on temporal and morphological indicators computed using the delineation (e.g., PR interval, RR interval, QT interval, QRS width, level of the ST segment, slope of the T-wave). Often times, the algorithms are designed by cardiologists. (Prineas et al., The Minnesota Code Manual of Electrocardiographic Findings, Springer, ISBN 978-1-84882-777-6, 2009). However, the current algorithms do not reflect the way the cardiologists analyze the ECGs and are crude simplifications. For example, the Glasgow University Algorithm does not reflect the way cardiologist analyze ECGs. (Statement of Validation and Accuracy for the Glasgow 12-Lead ECG Analysis Program, Physio Control, 2009.)

More advanced methods have also been developed that use learning algorithms. In. Shen et al., Biomedical Engineering and Informatics (BMEI), 2010. vol. 3, pp. 960-964, for instance, the author used support vector machines to detect bundle branch blocks. However, in these methods, once again, it is necessary to represent the raw data in a manner that preserves the invariance and stability properties.

While more complex neural network architectures have been proposed, limitations arose when they were applied to ECGs. One team (Jin and Dong, Science China Press, Vol. 45, No 3, 2015, pp 398-416; CN104970789) proposed binary classification on a full ECG, hence providing one and only one classification for any analyzed ECG. The proposed architecture used convolutional layers which processes the leads independently before mixing them into fully connected layers. The authors also mention multi-class analysis, as opposed to binary analysis, aiming at recovering one class among several. However, they did not consider multi-label classification, wherein multiple labels (e.g., abnormalities) are assigned to a cardiac signal.

Other algorithms and neural network architectures have been proposed to detect the risk of atrial fibrillation. In Attia et al., “An artificial intelligence-enabled ECG algorithm for the identification of patients with atrial fibrillation during sinus rhythm: a retrospective analysis of outcome prediction,” The Lancet, Volume 394, Issue 10201, P861-867, Sep. 7, 2019, the entire contents of which are incorporated herein by reference, the author describes using artificial intelligence and convolutional neural networks to detect asymptomatic atrial fibrillation.

In view of the foregoing limitations of previously-known systems and methods, it would be desirable to accurately and efficiently process ECG data and to present this information in a way that is easily comprehendible. For example, it would be desirable to use enhanced computing technology to analyze ECG data sampled from a patient to accurately and efficiently detect and/or predict cardiac events, e.g., using artificial intelligence and/or machine learning technology specifically designed for ECG analysis.

### SUMMARY OF THE INVENTION

Provided herein are systems and methods for analyzing ECG data using machine learning algorithms and medical grade artificial intelligence with enhanced accuracy and efficiency. Specifically, systems and methods are provided for analyzing electrocardiogram (ECG) data of a patient using artificial intelligence and a substantial amount of ECG data. The systems receive ECG data from a sensing device positioned on a patient such as one or more ECG leads/electrodes that may be integrated into smart technology (e.g., a smartwatch). The system may analyze ECG data sampled from the patient to accurately and efficiently detect and/or predict cardiac events such as such as cardiac arrhythmias and/or abnormalities including atrial fibrillation (AFib). The system may include an application that communicates with an ECG platform running on a server that processes and analyzes the ECG data, e.g., using neural networks for delineation of the cardiac signal and classification of various abnormalities, conditions and/or descriptors. The ECG platform may be a cloud-based ECG platform that processes and analyzes the ECG data in the cloud. The processed ECG data is communicated from the server for display in a user-friendly and interactive manner with enhanced accuracy. Together the ECG application and ECG platform implement the ECG processing system to receive ECG data, process and analyze ECG data, display ECG data on a system device, and generate a report having ECG data.

A computerized-system is provided herein for analyzing ECG data of a patient generated by one or more electrodes across a plurality of time points and comprising a plurality of beats. The computerized-system may be designed to analyze the ECG data using a delineation algorithm to generate wave information corresponding to a likelihood of a presence of at least one wave at the plurality of time points and further to determine beat onset information and beat offset information for beats of the plurality of beats where at least one wave is determined to be present to generate a plurality of beat onsets and beat offsets. The computerized system may further be designed to extract a plurality of beat portions of ECG data based on the plurality of beat onsets and beat offsets, each beat portion of the plurality of beat portions of ECG data corresponding to a beat of the plurality of beats, and determine that at least two beats of the plurality of beats should be grouped together based on the plurality of beat portions of ECG data, the at least two beats forming a cluster. Determining that the at least two beats of the plurality of beats should be grouped together may involve determining that the group data satisfies a threshold value.

The computerized-system may further be designed to analyze the plurality of portions of ECG data using an embedding algorithm to generate embedding data representative of the plurality of beats, and analyze the embedding data using a grouping algorithm to generate group data. The at least two beats of the plurality of beats may be determined to be grouped together based on the group data. The group data may correspond to a distance between two beats. The delineation algorithm may utilize a first neural network and the embedding algorithm may utilize a second neural network. The grouping algorithm may utilize a third neural network. The computerized-system may further be designed to receive user input data from an input device regarding an inaccuracy corresponding to displayed data related to the ECG data. The computerized-system may further be designed to adjust one or more of the delineation algorithm, embedding algorithm, or grouping algorithm based on the user input data.

The computerized-system may further be designed to modify the displayed data based on the user input data. The user input data may correspond to adding, deleting, or splitting one or more QRS clusters, PVC clusters, or PAC clusters. The embedding data may involve a vector of data for each beat of the plurality of beats. The computerized-system may further be designed to transmit information indicative of the cluster to a computer for display on a graphic user interface. The computerized-system may further be designed to generate information to display at least one overlay comprising at least two beats of the plurality of beats overlaid over one another. The computerized-system may further be designed to analyze the beats in the cluster using a classification algorithm to determine a likelihood of a presence of the one or more abnormalities, conditions, or descriptors associated with cardiac events for the patient.

The computerized-system may further be designed to analyze the wave information from the delineation algorithm using a classification algorithm to determine a likelihood of a presence of the one or more abnormalities, conditions, or descriptors associated with cardiac events for the patient. The wave information may be inputted into the classification algorithm and separately used to determine that at least two beats of the plurality of beats should be grouped together. The computerized-system may further be designed to, prior to analyzing the ECG data using the delineation algorithm, pre-process the ECG data to remove noise from the ECG data. The computerized-system may assign the ECG data and information based on the ECG data to a user account for review. The computerized may receive user input data regarding the ECG data and information based on the ECG data from the user account based on the review.

A method for analyzing electrocardiogram (ECG) data of a patient generated by one or more electrodes across a plurality of time points and comprising a plurality of beats is described herein. The method may involve analyzing the ECG data using a delineation algorithm to generate wave information corresponding to a likelihood of a presence of at least one wave at the plurality of time points, and determining beat onset information and beat offset information for beats of the plurality of beats where at least one wave is determined to be present to generate a plurality of beat onsets and beat offsets. The method may further involve extracting a plurality of beat portions of ECG data based on the plurality of beat onsets and beat offsets, each beat portion of the plurality of beat portions of ECG data corresponding to a beat of the plurality of beats, and determining that at least two beats of the plurality of beats should be grouped together based on the plurality of beat portions of ECG data, the at least two beats forming a cluster.

The method may further involve analyzing the plurality of portions of ECG data using an embedding algorithm to generate embedding data representative of the plurality of beats, and analyzing the embedding data using a grouping algorithm to generate group data. The at least two beats of the plurality of beats may be determined to be grouped together based on the group data. The method may further involve assigning the ECG data and information based on the ECG data to a user account for review of the ECG data. The method may further involve submitting the ECG data and information based on the ECG data for quality review by one or more reviewers. The method may further involve receiving quality control input generated by the one or more reviewers. The method may further involve causing display of the quality control input for additional quality control review. The method may further involving receiving user input data from an input device regarding an inaccuracy corresponding to information based on the ECG data. The method may further involve adjusting one or more of the delineation algorithm, embedding algorithm, or grouping algorithm based on the user input data. The method may further involve assigning the displayed data to a user account for quality review.

A system for analyzing ECG data of a patient may, in one example, involve a first plurality of instructions designed to, when executed, obtain ECG data of the patient over a plurality of time points and may further cause transmission of the ECG data to at least one server. The ECG data may be sampled at a predetermined sampling rate such as a rate of at least 20 samples per second. The system for analyzing ECG data may further involve a second plurality of instructions designed to, when executed, cause the at least one server to receive the ECG data of the patient, analyze the ECG data of the patient using at least one algorithm trained from a plurality of ECG data sets from different patients, quantify a likelihood of a presence of one or more abnormalities, conditions, or descriptors, or any combination thereof, and transmit information corresponding to the presence of the one or more abnormalities, conditions, or descriptors, or any combination thereof, to a computer remote from the at least one server for display.

The system for analyzing ECG data may further involve a third plurality of instructions designed to, when executed by the computer, cause the computer to display information corresponding the presence of the one or more abnormalities, conditions, or descriptors, or any combination thereof, based on the transmitted information from the at least one server. It is understood that each set of the plurality of ECG data sets from the different patients may be generated at a sampling rate equal to the rate used to obtain the ECG data. It is further understood that the computer that executes the third plurality of instructions may also execute the first plurality of instructions.

The second plurality of instructions may, when executed, further cause the at least one server to pre-process the ECG data which may involve removing noise from the ECG data or expressing the ECG data at a predetermined baseline frequency. Further, the second plurality of instructions, when executed, may analyze the ECG data of the patient using at least one algorithm that applies the ECG data to a first neural network for delineation and may further quantify a likelihood of a presence of at least one of a P-wave, QRS complex, or T-wave at each of the plurality of time points. The second plurality of instructions may further calculate at least one onset and at least one offset for at least one of the P-wave, QRS-complex, or T-wave, and/or calculate at least one measurement from one or more of the onset, the offset, or the output of the first neural network.

It is further understood that the second plurality of instructions may, when executed, analyze the ECG data of the patient using at least one algorithm that applies the ECG data to a second neural network for classification. Specifically, the second plurality of instructions may quantify a likelihood of a presence of the one or more abnormalities, conditions, or descriptors, and may apply a threshold to at least one value in the output of the second neural network and assign at least one label corresponding to the one or more abnormalities, conditions, or descriptors if the value exceeds a threshold. The second plurality of instructions may also post-process the ECG data by removing redundant labels.

The system may further include a fourth and/or fifth plurality of instructions. The fourth plurality of instructions may, when executed, cause the at least one server to generate a report including at least the transmitted information corresponding to the presence of the one or more abnormalities, conditions, or descriptors. The fifth plurality of instructions may, when executed, receive user input related to the ECG data and cause the computer to transmit the user input to the at least one server such that the at least one server uses the user input to generate the report. The report may include at least one heart rate density plot representing density of heart rates of the patient as a function of time. It is understood that a third plurality of instructions is further configured to, when executed by the computer, cause the computer to display a heart rate density plot representing density of heart rates of the patient as a function of time.

A system for analyzing ECG data of a patient may, in another example, involve instructions stored on at least one server that are designed to, when executed, cause the at least one server to receive a set of ECG data of the patient over a plurality of time points. The set of ECG data may be sampled at a predetermined sampling rate such as a rate of at least 20 samples per second. The instructions may further be designed to cause the at least one server to analyze the set of ECG data of the patient using at least one algorithm, quantify, at each time point of the plurality of time points, a likelihood of a presence of one or more abnormalities, conditions, or descriptors, or any combination thereof and transmit information corresponding to the likelihood of the presence of the one or more abnormalities, conditions, or descriptors to a computer for display. The at least one algorithm may be trained using a plurality of sets of ECG data generated at a sampling rate of at least 20 samples per second from different patients.

A computerized-method for analyzing ECG data of a patient may similarly involve receiving a set of ECG data of the patient over a plurality of time points sampled at a sample rate and analyzing the set of ECG data of the patient using at least one algorithm trained using a plurality of sets of ECG data. Each set in the plurality of sets of ECG data may be generated at the same sample rate from different patients. The computerized method for analyzing ECG data may further involve identifying, at each time point, one or more abnormalities, conditions or descriptors, or any combination thereof and further may involve transmitting information including the one or more abnormalities, conditions, or descriptors, or any combination thereof to a computer for display. It is understood that the computerized-method may involve analyzing an entire set of sampled ECG data without discarding data from the set of ECG data. The computerized-method may, in one example, involve a sample rate of at least 20 samples per second.

The computerized-method may further involve assigning the set of ECG data and information based on the set of ECG data to a user account for review of the ECG data. The computerized-method may further involve submitting the set of ECG data and information based on the set of ECG data for quality review by one or more reviewers. The computerized-method may further involve receiving quality control input generated by the one or more reviewers. The method may further involve causing display of the quality control input for additional quality control review.

A computerized-system for analyzing electrocardiogram (ECG) data of a patient may, in another example, include a computerized-system to analyze the ECG data to determine a presence of a cardiac event. If the cardiac event is determined to be present based on the analysis of the ECG data, the computerized-system may generate information to identify the presence of the cardiac event for display. If the cardiac event is determined not to be present based on the analysis of the ECG data, the computerized-system may further analyze the ECG data to determine a risk score indicative of future risk of the cardiac event for display. The cardiac event may be atrial fibrillation.

A computerized-system for analyzing ECG data of a patient may, in another example, analyze the ECG data using a delineation algorithm to determine a likelihood of a presence of at least one wave and may analyze the ECG data using a classification algorithm to extract a plurality of feature maps corresponding to the ECG data. The computerized-system may further apply the plurality of feature maps to a recurrent neural network and analyze the plurality of feature maps using the recurrent neural network to determine a sequence label corresponding to a first beat based, at least in part, on a feature map of the plurality of feature maps indicative of a second beat occurring immediately before the first beat. The sequence label may be one of ectopic, supraventricular, or PVC.

A computerized-system for analyzing ECG data of a patient may, in another example, analyze the ECG data using a delineation algorithm to determine wave information indicating a likelihood of a presence of at least one wave and analyze the ECG data and wave information using a baseline classification algorithm. The computerized-system may further determine a first value using the baseline classification algorithm, the first value indicating a presence of at least one cardiac event, and may analyze the ECG data and wave information using a desensitized classification algorithm, the desensitized classification algorithm having decreased sensitivity compared to the baseline classification algorithm. Additionally, the computerized-system may determine a second value using the desensitized classification algorithm, analyze the ECG data and wave information using a sensitive classification algorithm, the sensitive classification algorithm having increased sensitivity compared to the baseline classification algorithm, may determine a third value using the sensitive classification algorithm, and may determine that the baseline classification is certain based on the second value and the third value indicating the presence of the at least one cardiac event. The computerized-system may further automatically generate a report corresponding to the presence of the at least one cardiac event.

A computerized-system for analyzing ECG data of a patient may, in another example, upload ECG data to the computerized-system from a database of ECG data, assign a profile to the ECG data, determine instructions to associate a predetermined label with the ECG data, assign the predetermined label to the profile associated with the ECG data, and determine instructions to filter a plurality of ECG profiles based on the predetermined label, the plurality of profiles including the profile. The computerized-system may further analyze the ECG data to determine a presence of a cardiac event and assign a second label to the profile associated with the ECG data, the second label based on the presence of the cardiac event.

A computerized-system for analyzing ECG data of a patient may, in another example, determine a plurality of ECG data, the plurality of ECG data including first ECG data corresponding to a first lead and second ECG data corresponding to a second lead, cause an ECG interface to display a first graphical representation of at least a portion of the first ECG data, determine instructions to display a second graphical representation of at least a portion of the second ECG data in addition to the first graphical representation, and cause the ECG interface to simultaneously display the second graphical display synced in time with the first graphical display. The computerized-system may further determine third ECG data corresponding to a third lead, the plurality of ECG data further including the third ECG data, may determine instructions to display a third graphical representation of at least a portion of the third ECG data and the second ECG data, and may cause the ECG interface to simultaneously display the third graphical representation synced in time with the second graphical representation.

A computerized-system for analyzing ECG data of a patient may, in another example, analyze the ECG data using a delineation algorithm to determine first information indicating a likelihood of a presence of at least one wave and may analyze the ECG data and the first information using a plurality of classification neural networks. Each of the plurality of classification neural networks may utilize weighted values unique to its classification neural network. The computerized-system may further determine a plurality of outputs using the plurality of classification neural networks. Each output of the plurality of outputs may correspond to a classification neural network of the plurality of classification neural networks. The computerized-system may further analyze the plurality of outputs using a combiner to determine a probability of atrial fibrillation and a confidence score indicative of an accuracy of the probability of atrial fibrillation. The combiner may determines an average value by averaging the plurality of outputs. Alternatively, the combiner may determines a minimum value of the plurality of outputs. In another example, the combiner may determines a maximum value of the plurality of outputs.

A computerized-system for analyzing ECG data of a patient may, in another example, analyze the ECG data using a delineation algorithm to determine first information indicating a likelihood of a presence of at least one wave, may analyze the ECG data and first information using an input transformer to modify the ECG data and generate a plurality of inputs, and may analyze the plurality of inputs using a classification neural network. Further, the computerized-system may determine a plurality of outputs using the classification neural network. Each output of the plurality of outputs may correspond to an input of the plurality of inputs. Further, the computerized-system may analyze the plurality of outputs using a combiner to determine a probability of atrial fibrillation and a confidence score indicative of an accuracy of the probability of atrial fibrillation. The combiner may determine an average value by averaging the plurality of outputs. The combiner may determine a minimum value of the plurality of outputs. The combiner may determine a maximum value of the plurality of outputs. The input transformer may perform an amplification transformation to amplify the ECG data using a float value. The input transformer may perform a dilation transformation to warp the ECG data in time.

A computerized-system for analyzing electrocardiogram (ECG) data of a patient, may in another example, determine the ECG data indicative of at least one ECG event, parse the ECG data to determine that the ECG data is abnormal, and generate a report using the ECG data, the report indicating that the ECG data is abnormal. The computerized-system may further update an electronic medical record (EMR) using the ECG data, and determine billing information based on the report. It is understood that the term EMR used herein is interchangeable with the term electronic health records (EHR).

A computerized-system for analyzing electrocardiogram (ECG) data of a patient, may in another example, determine the ECG data indicative of at least one ECG event, parse the ECG data to determine that the ECG data is abnormal, and generate a report using the ECG data, the report indicating that the ECG data is abnormal. The computerized-system may further display the report in response to a request to view the report, determine that the report is a high priority, and receiving instructions to affix a signature to the report.

A computerized method for analyzing electrocardiogram (ECG) data of a patient may, in another example, include obtaining, from a first device, a set of patient ECG data corresponding to a patient, the set of patient ECG data generated over a first plurality of time points as sampled by a sensing device, obtaining, from a second device, a set of patient sensor data corresponding to the patient, the set of patient sensor data generated over a second plurality of time points, the second plurality of time points corresponding to the first plurality of time points, processing at least a portion of the set of patient ECG data and at least a portion of the set of sensor data using an algorithm to determine a presence of one or more abnormalities, conditions, or descriptors corresponding to a cardiac event associated with the set of patient ECG data and the set of patient sensor data, the algorithm trained using a plurality of sets of ECG data different from the set of ECG data and a plurality of sets of sensor data different from the set of patient sensor data, generating information, based on the processing, to indicate the presence of the one or more abnormalities, conditions, or descriptors corresponding to a cardiac event associated with the set of patient ECG data and set of patient sensor data, and sending the information corresponding to the presence of the one or more abnormalities, conditions, or descriptors determined for the set of patient ECG data and the set of patient sensor data for display.

The second device may be a photoplethysmogram (PPG) sensor. The patient sensor data may include one or more of heart rate, SpO2, respiratory rate data. The first device may be an implantable loop recorder (ILR). The computerized method may further include generating a database associating the ECG data with the first device and the patient sensor data with the second device. The computerized-method may further include obtaining, from the second device, a set of second sensor data corresponding to the patient and different than the set of patient sensor data. The set of second sensor data may be generated over a third plurality of time points corresponding to the first plurality of time points. The computerized-method may further include processing at least a portion of the set of second sensor data using the algorithm, wherein the algorithm maybe further trained using a plurality of sets of second sensor data different from the set of second sensor data.

A computerized- method for analyzing electrocardiogram (ECG) data of a patient may, in another example, include determining patient ECG data indicative of at least one cardiac event, processing at least a portion of the patient ECG data using an algorithm to determine a presence of one or more descriptors corresponding to the at least one cardiac event associated with the patient ECG data, the algorithm trained using a plurality of sets of ECG data different from the patient ECG data, determining a cardiac event and a descriptor corresponding to the cardiac event, generating an event interface indicating the descriptor and including a graphical representation of the cardiac event, and receiving input corresponding to the descriptor.

The input may reclassify the cardiac event as a second descriptor. The computerized-method may further include generating an event interface indicating the second descriptor and including a graphical representation of the cardiac event. The second descriptor may be used to train the algorithm. The event interface may further include one or more of heart rate information or event duration information.

A computerized-method for analyzing electrocardiogram (ECG) data of a patient, may in another example, include determining ECG history data, the ECG history data corresponding at least one arrhythmia event and sampled at a variety of time points processing ECG history data using an algorithm trained to determine a time point corresponding to a risk of an arrhythmia, determining a first time period associated with the risk of an arrhythmia, and sending a request for ECG data corresponding to the time period. The request for ECG data may be sent to a user's mobile device. The request for ECG data may be sent to a sensor device. The risk of an arrhythmia may be a risk of atrial fibrillation. The algorithm may be trained to determine a premature atrial contraction (PAC) burden.

A computerized-method for analyzing electrocardiogram (ECG) data of a patient may, in another example, include determining the ECG data indicative of at least one ECG event, processing ECG history data using an algorithm trained to determine at least one of a condition, descriptor or abnormality, determining a plurality of results corresponding to the at least one the condition, descriptor or abnormality, determining an indication associated with the patient, determining a prioritized order of the plurality of results based on the indication, and causing the prioritized order of the plurality of results to be presented on a computing device. The computerized-method may further include receiving a request to reprioritize the order of the plurality of results and determining a second prioritized order of the plurality of results based on the request to reprioritize. The plurality of results may include a first condition, and the computerized-method may further include determining an association between the indication and a first condition, and prioritizing the first condition based on the association.

A computerized-method for analyzing electrocardiogram (ECG) data of a patient may, in another example, include generating an ECG report comprising at least one ECG strip and at least one selectable feature designed to generate a request to access a viewer application, receiving the request to access the viewer application, the request associated with the at least one selectable feature, granting access to the viewer application, generating a viewer interface to be viewed using the viewer application, the viewer interface designed to present a heart rate density plot corresponding to the at least one ECG strip, receiving a request to perform an action on the viewer application, and determining to grant the request to perform the action. The computerized-method may further include requesting user credentials in response to the request to access the viewer application. Granting access to the viewer application may be based on the user credentials. The user credentials may correspond to a user profile and granting the request to perform the action may be based on the user profile. The action may be one or more of adding comments to the viewer application and modifying the ECG report.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1A is a recording of a standard 12-lead resting ECG and FIG. 1B is a recording of an exemplary P-wave, QRS complex and T-wave.

FIG. 2 is a diagram illustrating exemplary components for executing systems and methods in accordance with aspect of the present disclosure.

FIGS. 3A-3B are schematic views of the exemplary hardware and software components of an exemplary system device and an exemplary server, respectively.

FIG. 4 is a flow chart of an exemplary method of processing ECG data using, displaying ECG data, and generating a report including ECG data.

FIGS. 5A-5B are line graphs representing an exemplary ECG signal and an exemplary output of a first neural network for each wave type analyzed, respectively.

FIGS. 6A-6B are exemplary representations of classification neural networks in the form of a convolutional neural network and a recurrent neural network, respectively.

FIG. 7 is an exemplary representation of a variable number of lead entries and a constant number of outputs.

FIG. 8 is an exemplary user interface having a heart rate density plot generated in accordance with aspects of the recent disclosure.

FIG. 9 is a zoomed-in view of the heart rate density plot shown in FIG. 8.

FIG. 10 is an exemplary user interface having a heart rate density plot generated in accordance with aspects of the present disclosure.

FIG. 11 is a flow chart illustrating an exemplary approach for generating a heart rate density plot.

FIG. 12 is an exemplary heart rate density plot generated in accordance with aspects of the present disclosure.

FIG. 13 is an exemplary user interface having a zoomed-in heart rate density plot.

FIGS. 14A-14E are side-by-side comparisons of various R-R plots and heart rate density plots generated from the same cardiac signal.

FIGS. 15A-15D is an exemplary report generated by the ECG processing system having information corresponding to the patient and processed ECG data and displaying a heart rate density plot and ECG strips.

FIG. 16 illustrates an exemplary process flow for determining ECG data and associating the ECG data to a user profile.

FIGS. 17A-17B illustrate an exemplary process and data flow for determining ECG data, parsing the ECG data, and determining reports based on the ECG data.

FIG. 18 illustrates an exemplary process flow for determining ECG data, determining a report, prioritizing the report, and signing the report.

FIGS. 19A-19C illustrate an exemplary ILR event monthly summary report.

FIG. 20 illustrates an exemplary ILR event report.

FIGS. 21A-21C illustrate an exemplary monthly report and events list user interface.

FIGS. 22A-22B illustrate exemplary user registration and profile interfaces.

FIG. 23A illustrates an exemplary event interface including a reclassification menu. FIG. 23B illustrates an exemplary process for reclassifying an event.

FIG. 24 illustrates color bands that may be displayed on an event interface.

FIG. 25A is a diagram illustrating an exemplary multi-user device system for analyzing ECG and other data. FIG. 25B is a process for analyzing ECG data and other data to determine an anomaly, descriptor, or condition using multiple user devices.

FIG. 26 illustrates an exemplary mobile device interface for presenting ECG data and results.

FIG. 27 is an exemplary process for prioritizing certain data for review by the healthcare provider based on a user indication.

FIG. 28. is an exemplary process for determining a time period for which an arrhythmia is likely and determining ECG data during that time period.

FIG. 29. is an exemplary process for determining a time period for which atrial fibrillation is likely based on the PAC burden and determining ECG data during that time period.

FIGS. 30A-30B illustrate an events report including a graphical representation of events detected.

FIGS. 31A-31F illustrate various user interfaces for displaying patients, indications, classifications and/or events.

FIG. 32 is a portion of an ECG report including selectable ECG strips and selectable links to be redirected to a viewer application.

FIG. 33 illustrates a viewer interface of a viewer application including a heart rate density plot and ECG strips.

FIG. 34 is an exemplary process for redirecting a user from the report to a viewer application including a viewer interface.

FIGS. 35A-35C are exemplary report, patients and event list interfaces.

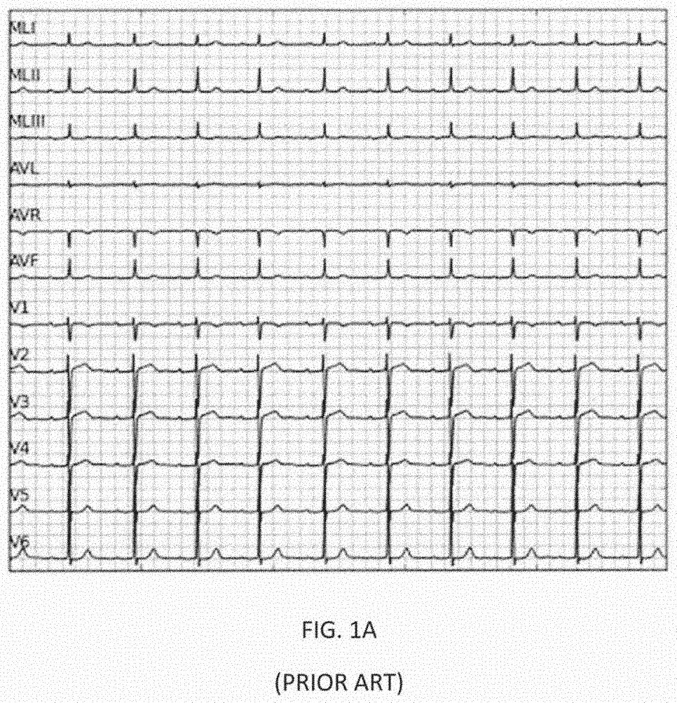

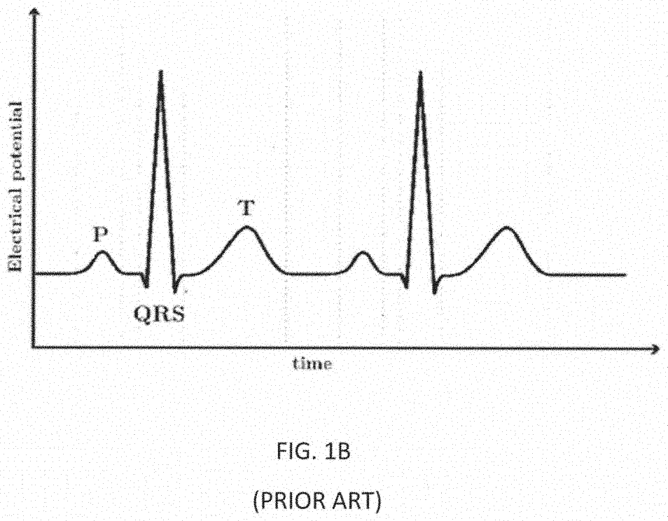

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION OF THE INVENTION

The present invention is directed to an electrocardiogram (ECG) processing system having medical grade artificial intelligence involving an ECG application run on a system device and an ECG platform run on a server(s). The ECG application and ECG platform implement the ECG processing system by processing and analyzing the ECG data using machine learning algorithms to detect and/or predict cardiac events such as such as cardiac arrhythmias and/or abnormalities including atrial fibrillation (AFib). The system may achieve delineation of the cardiac signal and classification of various abnormalities, conditions, and descriptors. The server(s) may be located in a different location than the system device(s) and the servers need not be in the same physical location as one another (e.g., the server(s) may be a remote server(s)). Alternatively, the server(s) and the system device(s) may be located in the same general area (e.g., on a local area network (LAN)). The ECG platform may be a cloud-based ECG platform that may implement the ECG processing system by processing and analyzing the ECG data in the cloud.

To implement the ECG processing system, ECG application running on the system device may receive ECG data (i.e., cardiac signal) from a sensing device and may transmit the ECG data to a ECG platform running on the server. The ECG platform may execute a first and second neural network and may apply the ECG data to the first and second neural network. The first neural network may be a delineation neural network having machine learning functionality. The second neural network may be a classification neural network having machine learning functionality. The output of the first and/or second neural networks may be processed by the ECG platform to achieve delineation and classification of the ECG data. The ECG data and/or data generated by the ECG platform may be communicated from the ECG platform to the ECG application. The ECG application may cause the ECG data and/or data generated by the ECG platform to be displayed in an interactive manner. The ECG platform may generate reports including ECG data and/or data generated by the ECG platform, and may communicate the reports to the ECG application.

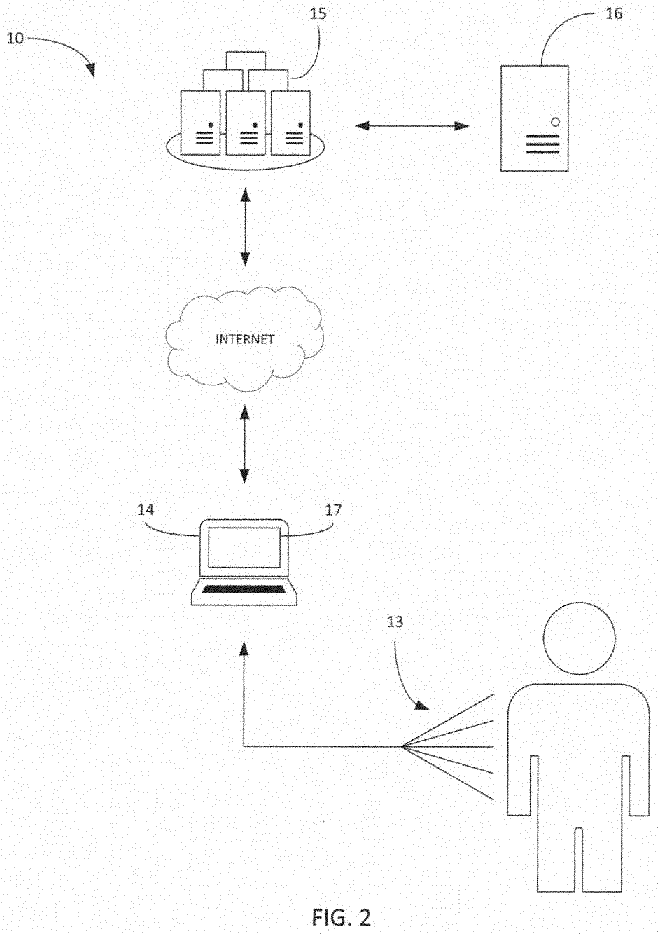

Referring now to FIG. 2, exemplary components for executing electrocardiogram (ECG) processing system 10 are illustrated. FIG. 2 shows ECG sensing device 13, system device 14, and server 15, as well as drive 16.

ECG sensing device 13 is designed to sense the electrical activity of the heart for generating ECG data. For example, sensing device 13 may be one or more electrodes that may be disposed on one or more leads. ECG sensing device 13 may be an ECG-dedicated sensing device such as a conventional 12-lead arrangement or may be a multi-purposes device with sensing hardware for sensing electrical activity of the heart for ECG generation such as the Apple Watch available from Apple, Inc., of Cupertino, Calif. Sensing device 13 may be placed on the surface of the chest of a patient and/or limbs of a patient. Sensing device 13 may be in electrical communication with system device 14 running the ECG application 29 such that the electrical signal sensed by sensing device 13 may be received by the ECG application 29. ECG application 29 may include instructions that cause sensing device 13 to sense or otherwise obtain ECG data.

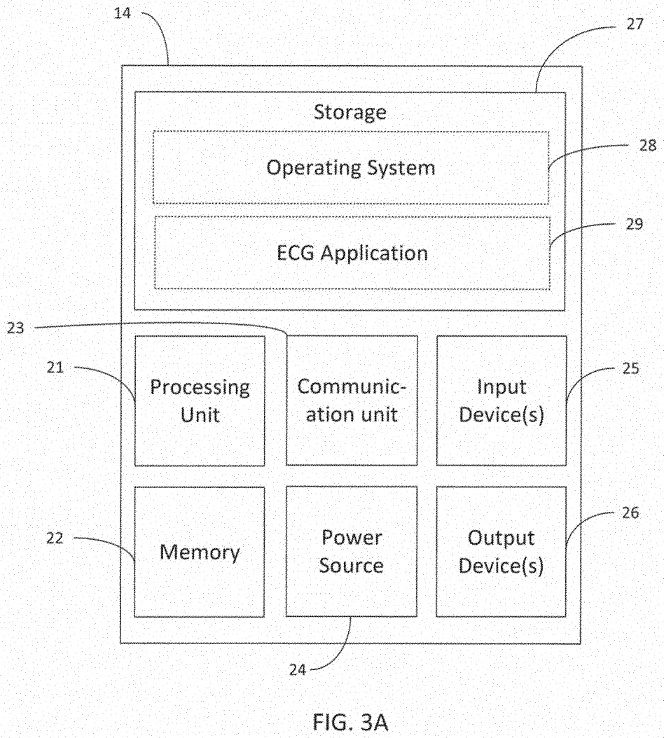

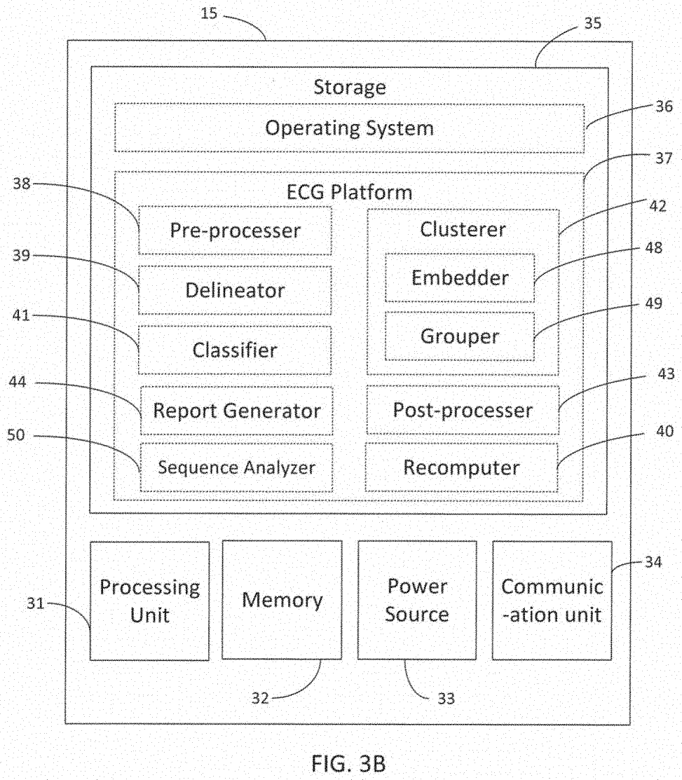

System device 14 is preferably one or more computing devices (e.g., laptop, desktop, tablet, smartphone, smartwatch, etc.) having the components described below with reference to FIG. 3A and the functionality described herein. System device 14 running ECG application 29 may connect with server 15 running ECG platform 37 via any well-known wired or wireless connection. For example, system device 14 may connect to the Internet using well known technology (e.g., WiFi, cellular, cable/coaxial, and/or DSL) and may communicate with server 15 over the Internet.

Server 15 is preferably one or more servers having the components described below with reference to FIG. 3B and the functionality described herein. Server 15 preferably has processing power superior to system device 14 such that server 15 can process and analyze cardiac signals having a sampling rate above a predetermined threshold, such as at least 20 samples per second, at least 250 samples per second, or at least 1000 samples per second. As will be readily apparent to one skilled in the art, server 15 may include a plurality of servers located in a common physical location or in different physical locations. In a preferred embodiment, server 15 is located in a different, remote location (e.g., on the cloud) than system device 14, although server 15 and system device 14 may be located in a common location (e.g., on a local area network (LAN)).

Server 15 may optionally communicate with drive 16 which may be one or more drives having memory dedicated to storing digital information unique to a certain patient, professional, facility and/or device. For example, drive 16 may include, but is not limited to, volatile (e.g. random-access memory (RAM)), non-volatile (e.g. read-only memory (ROM)), flash memory, or any combination thereof. Drive 16 may be incorporated into server 15 or may be separate and distinct from server 15 and may communicate with server 15 over any well-known wireless or wired connection.

Aspects of ECG processing system 10 and/or any other ECG processing systems described throughout this application may be the same or similar to the ECG processing system described in WO2020161605A1, which is the published application of PCT/IB2020/050850, filed on Feb. 3, 2020, (corresponding to U.S. Ser. No. 17/390,714), which claims priority to U.S. Pat. No. 10,959,660 to Li, the entire contents of each of which are incorporated herein by reference. Additional technology that may be utilized is described in commonly-assigned U.S. Ser. No. 17/397,782, the entire contents of which are incorporated herein by reference.

Referring now to FIGS. 3A-3B, exemplary functional blocks representing the hardware and software components of system device 14 and server 15 are shown. Referring now to FIG. 3A, hardware and software components of system device 14 may include one or more processing unit 21, memory 22, storage 27, communication unit 23, and power source 24, input devices 25 and output devices 26.

Processing unit 31 may be one or more processors configured to run collaboration operating system 28 and ECG application 29 and perform the tasks and operations of system device 14 set forth herein. Memory 22 may include, but is not limited to, volatile (e.g. random-access memory (RAM)), non-volatile (e.g. read-only memory (ROM)), flash memory, or any combination thereof. Communication unit 23 may receive and/or transmit information to and from other components in ECG processing system 10 including, but not limited to, sensing device 13 and server 15. Communication unit 23 may be any well-known communication infrastructure facilitating communication over any well-known wired or wireless connection, including over any well-known standard such as any IEEE 802 standard. Power source 24 may be a battery or may connect system device 14 to a wall outlet or any other external source of power. Storage 27 may include, but is not limited to, removable and/or non-removable storage such as, for example, magnetic disks, optical disks, or tape.

Input device 25 may be one or more devices coupled to or incorporated into system device 14 for inputting data to system device 14. Input device 25 may further include a keyboard, a mouse, a pen, a sound input device (e.g., microphone), a touch input device (e.g., touch pad or touch screen), a location sensor, and/or a camera, for example. Output device 26 may be any device coupled to or incorporated into system device 14 for outputting or otherwise displaying data and includes at least a display 17. Output device 26, may further include speakers and/or a printer, for example.

ECG application 29 may be stored in storage 27 and executed on processing unit 21. ECG application 29 may be a software application and/or software modules having one or more sets of instructions suitable for performing the operations of system device 14 set forth herein, including facilitating the exchange of information with sensing device 13 and server 15. For example, ECG application 29 may cause system device 14 to receive ECG data from sensing device 13, to record ECG data from sensing device 13, to communicate ECG data to server 15, to instruct server 15 to process and analyze ECG data, to receive processed and/or analyzed ECG data from server 15, to communicate user input regarding report generation to server, and to generate a graphic user interface suitable for displaying raw, analyzed and/or processed ECG data and data related thereto.

Operating system 28 may be stored in storage 27 and executed on processing unit 21. Operating system 28 may be suitable for controlling the general operation of system device 14 and may work in concert with ECG application 29 to achieve the functionality of system device 14 described herein. System device 14 may also optionally run a graphics library, other operating systems, and/or any other application programs. It of course is understood that system device 14 may include additional or fewer components than those illustrated in FIG. 3A and may include more than one of each type of component.

Referring now to FIG. 3B, hardware and software components of server 15 may include one or more processing unit 31, memory 32, storage 35, power source 33, and communication unit 34. Processing unit 31 may be one or more processors configured to run operating system 36 and ECG platform 37 and perform the tasks and operations of server 15 set forth herein. Given the volume of data and processing tasks assigned to processing unit 31, it is understood that processing unit 31 has superior processing power compared to processing unit 21.

Memory 32 may include, but is not limited to, volatile (e.g. random-access memory (RAM)), non-volatile (e.g. read-only memory (ROM)), flash memory, or any combination thereof. Storage 35 may include, but is not limited to, removable and/or non-removable storage such as, for example, magnetic disks, optical disks, or tape. Communication unit 34 may receive and/or transmit information to and from other components of ECG processing system 10 including, but not limited to, system device 14 and/or drive 16. Communication unit 34 may be any well-known communication infrastructure facilitating communication over any well-known wired or wireless connection. Power source 33 may be a battery or may connect server 15 to a wall outlet or other external source of power.

Operating system 36 and ECG platform 37 may be stored in storage 35 and executed on processing unit 31. Operating system 36 may be suitable for controlling general operation of server 15. ECG platform 37 may be a software application and/or software modules having one or more sets of instructions. ECG platform 37 may facilitate and oversee the processing and analysis of ECG data received from system device 14, report generation, and otherwise may be suitable for performing the operations of server 15 set forth herein.

ECG platform 37 may include several sub-modules and/or applications including, but not limited to, pre-processor 38, delineator 39, classifier 41, clusterer 42 which may include embedder 48 and grouper 49, post-processor 43, report generator 44, recomputer 40 and/or sequence analyzed 50. Each sub-module and/or application may be a separate software application and/or module having one or more sets of instructions. Pre-processor 38 may pre-process raw ECG data, delineator 39 may execute a first neural network to achieve delineation, classifier 41 may execute a second neural network to achieve classification, clusterer 42 may identify clusters in data processed by the first neural network, post-processor 43 may post-process data processed by the second neural network, embedder 48 may execute one or more algorithms and/or a third neural network to achieve embedding, grouper 49 may execute one or more algorithms and/or a fourth neural network to generate cluster groups, report generator 44 may generate reports based on raw ECG data and ECG data processed by ECG platform 37, and recomputer 40 may recompute and/or adjust embedder 48 and/or grouper 49 based on user input data. For example, recomputer 40 may recalculate episodes based on corrected wave information. Sequence analyzer 50 may be one or more algorithms and/or a third neural network which may be a recurrent neural network. Sequence analyzer 50 may analyze feature maps to determine one or more sequence labels and thereby achieve sequence identification as explained below. ECG platform 37 may also perform various other functions including, but not limited to, receiving requests from system device 14 to process and/or analyze ECG data, communicating processed and/or analyzed ECG data to system device 14, receiving a request to generate a report, requesting and/or receiving user interaction and/or instructions from system device 14, receiving user input data and/or instruction information from system device 14 regarding report generation, and/or communicating a report to system device 14.

Server 15 may also optionally run a graphics library, other operating systems, and/or any other application programs. It of course is understood that server 15 may include additional or fewer components than those illustrated in FIG. 3B and may include more than one of each type of component.

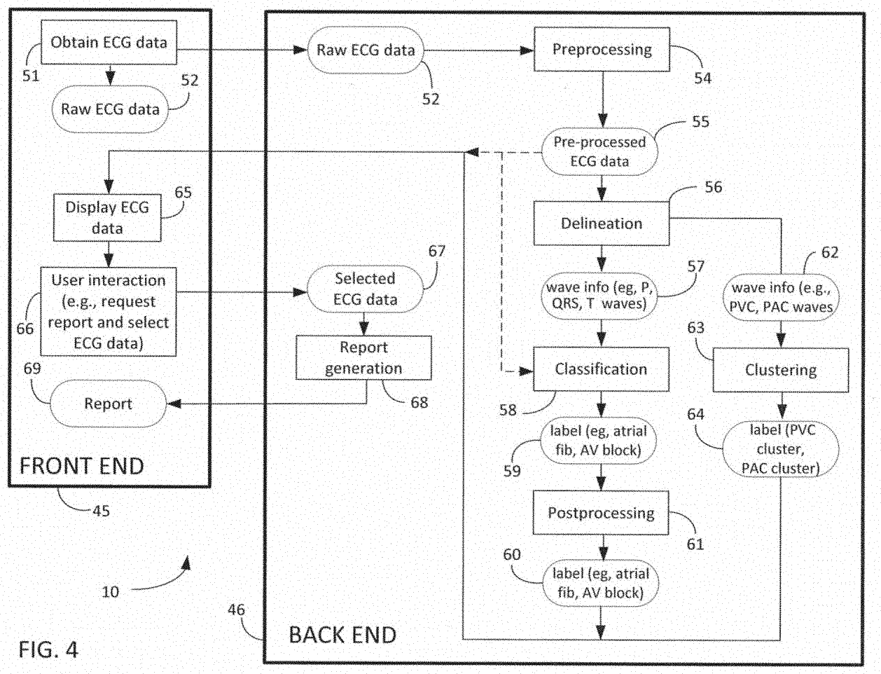

FIG. 4 illustrates an exemplary process for implementing ECG processing system 10 to receive and record ECG data, process and analyze ECG data, and generate reports involving ECG data, and further shows the flow of information between front end 45 and back end 46 of ECG processing system 10, as described, for example, in U.S. Patent Pub. Nos. 2019/0167143, U.S. Patent Pub. No. 2019/0223739, and U.S. Pat. No. 10,426,364, the entire contents of each of which are incorporated herein by reference. Front end 45 includes at least ECG application 29 running on system device 14. Back end 46 includes at least ECG platform 37 running on server 15.

As is shown in FIG. 4, at step 51, ECG application 29 may cause system device 14 to receive and/or otherwise obtain raw ECG data 52 from sensing device 13. For example, ECG application 29 may cause sensing device 13 to sense the cardiac signal and communicate the cardiac signal sensed by sensing device 13 to system device 14. Raw ECG data is the cardiac signal sensed by sensing device 13. Raw ECG data 52 has not been processed or analyzed by ECG processing system 10. Raw ECG data 52 preferably involves data sampled multiple times per heartbeat over a plurality of heartbeats. It is understood that sensing device 13 may convert an analog cardiac signal into a digital signal, a different component not shown in FIG. 2 may convert the analog cardiac signal to a digital signal, or ECG application 29 may cause system device 14 to convert the analog cardiac signal to a digital signal. Raw ECG data in both analog and digital form are referred to herein as raw ECG data 52.

Upon receiving raw ECG data 52, ECG application 29 may cause system device 14 to record raw ECG data 52 and may optionally save some or all of raw ECG data 52 to system device 14. As explained above, the signals may correspond to one or more leads. When multiple leads are used, all leads may be processed simultaneously. It is understood that the cardiac signal generated by each lead may have varying lengths. It is further understood that the cardiac signal may be short term (e.g., 10 seconds in standard ECGs) or long term (several days in holters). System device 14 may optionally display raw ECG data 52 or a portion thereof on display 17.

As is shown in FIG. 4, raw ECG data 52 may be transmitted from front end 45 to back end 46. Specifically, ECG application 29 may cause system device 14 to communicate raw ECG data 52 to ECG platform 37 running on server 15. Upon receiving raw ECG data 52, ECG platform 37 may cause server 15 to save some or all of raw ECG data 52 to server 15. Further, after receiving raw ECG data 52, ECG platform 37 cause raw ECG data 52 to be preprocessed at step 54 by pre-processor 38. It is understood that pre-processor 38 may be a stand-alone component of ECG platform 37 or subcomponent of delineator 39.

Pre-processor 38 may process raw ECG data 52 or a portion thereof by removing the disturbing elements of the cardiac signal, such as noise from the raw ECG data. For noise filtering, a multivariate functional data analysis approach may be used (Pigoli and Sangalli. Computational Statistics and Data Analysis, Vol. 56, 2012, pp 1482-1498). As the signal sensed by sensing device 13 may vary due to a patient's movements, the baseline frequency of raw ECG data 52 may be removed by pre-processor 38 and the cardiac signal may be expressed at a chosen frequency. The frequencies of the signal corresponding to the patient's movements may be removed using median filtering (Kaur et al., Proceedings published by International Journal of Computer Applications, 2011, pp 30-36). Applying raw ECG data 52 to pre-processor 38 generates pre-processed ECG data 55. At this point, ECG platform 37 may cause pre-processed ECG data 55 to optionally be communicated to ECG application 29 running on system device 14 for display on display 17. ECG platform 37 may alternatively, or additionally, cause pre-processed ECG data 55 to be used as an input at classification step 58, discussed in more detail.

At step 56, ECG platform 37 causes pre-processed ECG data 55 to be applied to delineator 39 for delineation. Delineator 39 applies a first neural network that is a delineation neural network to pre-processed ECG data 55. A neural network refers to a mathematical structure or algorithm that may take an object (e.g., matrix or vector) as input and produce another object as an output though a set of linear and non-linear operations called layers. For example, the input of the first neural network may be one or more multi-lead cardiac signals that are pre-processed to remove noise and/or baseline wandering.

To apply pre-processed ECG data 55 to the first neural network, delineator 39 may cause some or all of raw ECG data 52 to be expressed as matrix X, which may be a matrix of real numbers. For example, matrix X may be a matrix of size m×n at the frequency used for training the networks, described in more detail below. The constant “m” may be a number of leads in sensing device 13, which is typically 12, though any number of leads may be used. In this example, the number of samples “n” provides the duration of the cardiac signal “n/f” with f being the sampling frequency of the cardiac signal. The sample rate is above a predetermined rate and is preferably relatively high, such as, for example, at least 20, at least 250, at least 500 or at least 1000 samples per second, etc. In one embodiment, all of the sampled ECG data is transferred to the server for input into the processing algorithms without filtering out ECG data. While the ECG data applied to the first neural network is preferably pre-processed ECG data 55, it is understood that a non-preprocessed cardiac signal (i.e., raw ECG data 52, or a portion thereof) may be applied to the first neural network.

The first neural network may provide as an output, values corresponding to the likelihood of the presence of or one or more waves at a plurality of time points in the cardiac signal. The time points may be dictated by the raw ECG data, may be selected by the user of system device 14, or may be preprogrammed. The first neural network may be a convolutional neural network, and is preferably a fully convolutional neural network. Convolutional neural networks are a particular type of neural network where one or more matrices, which are learned, do not encode a full linear combination of the input elements, but the same local linear combination at all the elements of a structured signal, such as a cardiac signal, through a convolution (Fukushima, Biol. Cybernetics, Vol. 36, 1980, pp 193-202, LeCun et al., Neural Computation, Vol. 1, 1989, pp 541-551). A network which only contains convolutional networks is called a fully convolutional neural network.

Accordingly, at step 56, delineator 39 causes the first neural network to read each time point of the cardiac signal, spatio-temporally analyze each time point of the cardiac signal, and assign a score at each time point corresponding to one or more types of waves. In this manner, all types of waves in the cardiac signals may analyzed and the likelihood of their presence at each time point, quantified, in a single step. Accordingly, each score generated by delineator 39 is indicative of the likelihood of the presence of a particular wave type at a given time point of the cardiac signal. The wave types may be any well know wave type such as, P-waves, Q-wave, R-wave, S-wave, Q-waves, R-waves, S-waves, QRS complexes, and/or T-waves, for example. In this manner, delineator 39 may process data sampled multiple times per heart beat across a plurality of heart beats.

The output of the first neural network may be a matrix Y, which may be a matrix of real numbers. For example, matrix Y may be a matrix of the size p×n. Matrix Y may include scores for each type of wave at each time point of the cardiac signal. In matrix Y, “n” is the number of samples, as discussed above with respect to Matrix X, and “p” is the number of wave types plus the number of wave characterizations. As explained in more detail below, wave characterization may correspond to conductivity, prematurity, ectopy, and/or origin of the waves in the cardiac signal, for example. In one example, the wave types include (1) P-waves, (2) QRS complexes, and (3) T-waves, and the wave characterizations include (1) premature waves, (2) paced waves, (3) ventricular QRS complexes, (4) junctional QRS complexes, (5) ectopic P waves, and (6) non-conducted P waves. Accordingly, in this example, p=3+6=9. Each wave type may be expressed according to certain characteristics of that wave, such as start and end points (i.e., onset and offset)).

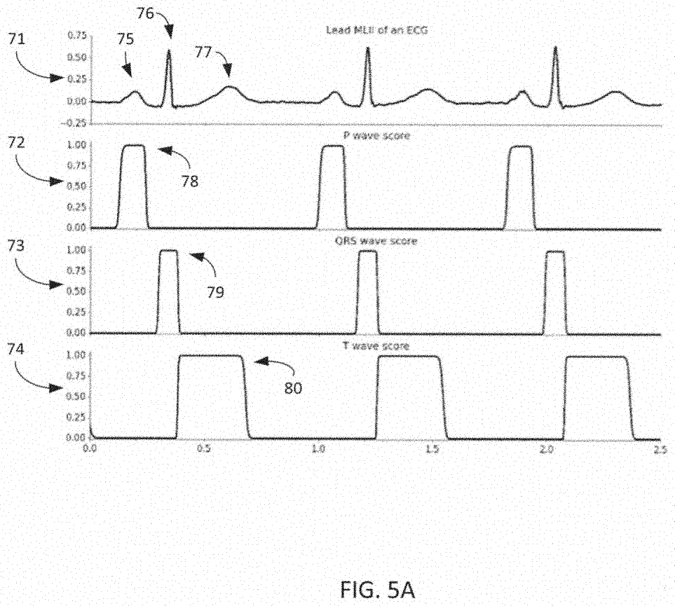

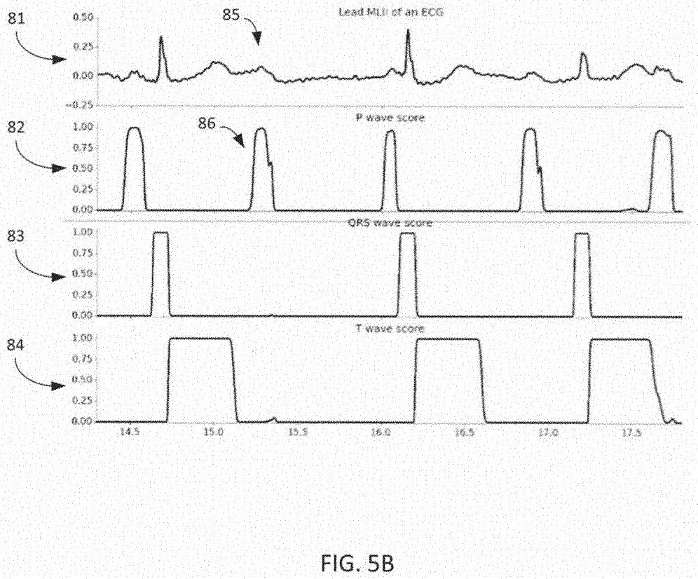

Referring now to FIGS. 5A and 5B, exemplary outputs of the first neural network are graphed for each wave type to illustrate the value of generating scores at each time point corresponding to a plurality of types of waves. Specifically, FIG. 5A illustrates an exemplary output where the delineation neural network processed a normal cardiac signal (with no abnormalities) and FIG. 5B illustrates an exemplary output where the delineation neural network processed a cardiac signal having “hidden” P-waves, for example due to an atrioventricular block.

Referring now to FIG. 5A, four line graphs are illustrated, each graph showing time on the x-axis. Line graph 71 represents the cardiac signal over multiple beats. The plotted signal reflects the well-known ECG waveform having a P-Wave (point 75), QRS complex (point 76), and T-wave (point 77). Line graph 72 is a graph the P-wave score over the same time points in the cardiac signal. Similarly, line graph 73 and line graph 74 are graphs of the QRS score and T-wave scores, respectively, over the same time points. The y-axis for each line graphs 72-74 is the score assigned at each time point, ranging from 0 to 1, with 0 indicating a low likelihood of the presence of a particular wave and 1 indicating a high likelihood of the presence of a particular wave. For example, line graph 72 indicates a very high likelihood of the presence of P-waves at score 78 which corresponds to the time points near point 75, line graph 73 indicates a very high likelihood of the presence of a QRS complex at score 79 which corresponds to the time points near point 76, and line graph 74 indicates a very high likelihood of the presence of a T-wave at score 80 which corresponds to the time points near point 77.

FIG. 5B, like FIG. 5A, illustrates four line graphs, line graphs 81-82, which are similar to line graphs 71-74. Specifically, line graph 81 represents the cardiac signal over several beats, line graph 82 represents the P-wave score over the cardiac signal, line graph 83 represents the QRS score over the cardiac signal, and line graph 84 illustrates the T-wave score over the cardiac signal. Unlike FIG. 5A, the ECG signal in line graph 81 includes hidden P-waves such as the hidden P-wave shown at point 85. Hidden P-waves are P-waves that occur during another wave or complex such as a T-wave. As the cardiac signal processed by the delineation network involves a high sample rate and the delineation network generates data for each wave type at each time point, the output recovered is robust enough (i.e., includes enough sample points) to identify two waves occurring at the same time, such as the case with hidden P-waves. For example, line graph 82 indicates a very high likelihood of the presence of P-waves at score 86 which corresponds to the time points near point 85. Accordingly, it is understood that the delineation neural network is not limited to recovering only one wave at each time point and therefore can identify several waves at any time point. It is further understood that signals from one or more leads may be processed simultaneously by the first neural network.

Using the scores assigned to each time point corresponding to each wave type (e.g., P-wave, QRS complex, T-wave, etc.), delineator 39 may post-process the cardiac signal. Post-processing involves, assigning to each time point, none, one, or several waves, calculating the onset and offset of each of the identified waves, and optionally determining the characterization of the waves. Waves may be assigned to each time point by determining that a wave exists at that time point if a certain value is achieved. Computing the “onset” and “offset” of each wave involves computing the time points of the beginning and the end of each wave in the cardiac signal, the beginning referred to as the “onset” and the end referred to as the “offset.” This may involve analyzing the time points corresponding begging and end of the highest values for each wave type. Delineator 39 may characterize the waves by identifying prematurity, conductivity and ectopy. Wave characterization leverages the contextual information between each wave and/or each beat. For example, the premature label may be applied to the wave if a certain threshold value is achieved at a certain time point or an average value over several time points.

After computing the onset and offset of each wave type in the cardiac signal, delineator 39 may calculate global measurements. Global measurements are derived from the onset and offset of each wave type and may relate to features and characteristics of the cardiac signal such as intervals between waves and wave durations. For example, global measurements may include, but are not limited to, PR interval, P-wave duration, QRS complex duration, QRS axis, QT interval, corrected QT interval (Qtc), T-wave duration, JT interval, corrected JT interval, heart rate, ST elevation, Sokolov index, number of premature ventricular complexes, number of premature atrial complexes (PAC), ratio of non-conducted P waves, and/or ratio of paced waves.

Delineator 39 may further deduce labels solely from the information generated by delineator 39. For example, the following labels may be deduced by delineator 39: short PR interval (i.e., PR interval<120 ms), first degree AV block (e.g., PR interval>200 ms), axis deviations, long QTc, short QTc, wide complex tachycardia, and/or intraventricular conduction blocks. Labels determined solely from information generated by delineator 39 are referred to as delineation based labels.

Referring again to FIG. 4, ECG platform 37 may cause the output of step 56 (e.g., wave information 62) as well as pre-processed ECG data 55 to be communicated or otherwise applied to clusterer 42 for clustering at step 63. Wave information 62 may include scores regarding PVC waves and PAC waves including onsets and offsets generated and relevant durations. Clusterer 42 may process wave information 62 and identify clusters of PAC or PAV waves during the duration of the cardiac signal. Once identified, clusterer 42 may assign cluster label 64 to one or more time windows, identifying either a PVC or a PAC cluster for each time window. A time window is defined by two time points in the cardiac signal.

Referring again to FIG. 4, ECG platform 37 may also cause the output of step 56 (e.g., wave information 57) as well as pre-processed ECG data 55 to be communicated or otherwise applied to classifier 41 for classification at step 58. Classification at step 58 involves applying a second neural network (i.e., classification neural network) to pre-processed ECG data 55. Accordingly, in one example, the input of the second neural network may be one or more multi-lead cardiac signals with variable length that is pre-processed. Classifier 41 may also process wave information 57 and/or other information such as patient-specific information including the patient's age or any relevant clinical information. As explained above, ECG platform 37 may cause optionally cause pre-processed ECG data 55 to be communicated directly to classifier 41 and processed by classifier 41 if delineation at step 56 is not necessary. In this manner, classifier 41 may process data sampled multiple times per heart beat across a plurality of heart beats.

The second neural network generates an output having values that correspond to the likelihood of the presence of one or more abnormality, condition and/or descriptor at each time point of the cardiac signal. If a time point or time window is determined to correspond to a certain abnormality, condition, and/or descriptor, a label corresponding to that abnormality, condition, and/or descriptor will be assigned to that time point or window. In one example, one or more labels 59 may be assigned to a time point or time window if a score achieves a predetermined threshold. Accordingly, multi-label localization may be achieved for abnormalities, conditions, and/or descriptors by generating a plurality of values at each time point and assigning one or more labels at each time point.

Classifier 41 may recover the output of the classification neural network as a vector of size q. The values in the vector correspond to the presence of each label at each time point or each time window. For example, the output of the classification neural network may be the vector [0.98: 0.89; 0.00] with the corresponding labels for each element of the vector: Right Bundle Branch Bloc; Atrial Fibrillation; Normal ECG. The scores may be between 0 and 1. For the vector above, a threshold of 0.5 would result in the labels “Right Bundle Branch Block” and “Atrial Fibrillation” being assigned by classifier 41 to the time point or time window corresponding to the score. It is understood that the threshold may be preprogrammed and/or selected by the user and may be modified to provide varying degrees of sensitivity and specificity. By assigning one or more labels for each time point, onsets and offsets corresponding to each label may be computed to identify durations of episodes (e.g., abnormalities episodes).

Abnormalities and conditions may include any physiological abnormality or condition which may be identifiable on the cardiac signal. Today about 150 measurable abnormalities may be identified on cardiac signal recordings. Abnormalities and conditions may include but are not limited to, sinoatrial block, paralysis or arrest, atrial fibrillation, atrial flutter, atrial tachycardia, junctional tachycardia, supraventricular tachycardia, sinus tachycardia, ventricular tachycardia, pacemaker, premature ventricular complex, premature atrial complex, first degree atrio-ventricular block (AVB), 2nd degree AVB Mobitz I, 2nd degree AVB Mobitz II, 3rd degree AVB, Wolff-Parkinson-White syndrome, left bundle branch block, right bundle branch block, intraventricular conduction delay, left ventricular hypertrophy, right ventricular hypertrophy, acute myocardial infarction, old myocardial infarction, ischemia, hyperkalemia, hypokalemia, brugada, and/or long QTc. Descriptors may include descriptive qualities of the cardiac signal such as “normal” or “noisy ECG.”

Upon applying the second neural network at step 58, classifier 41 may read each time point of the cardiac signal as well as each global measurement, analyze each time point of the cardiac signal and each global measurement, compute time windows by aggregating at least two time points, and compute scores for each time window, the scores corresponding to a plurality of non-exclusive labels.

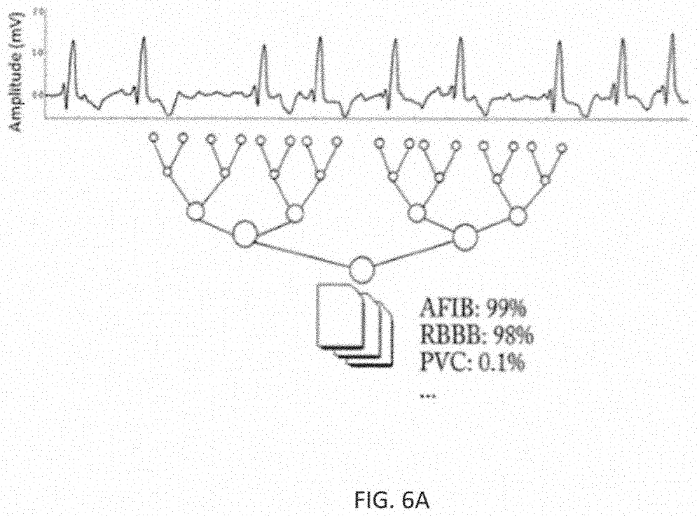

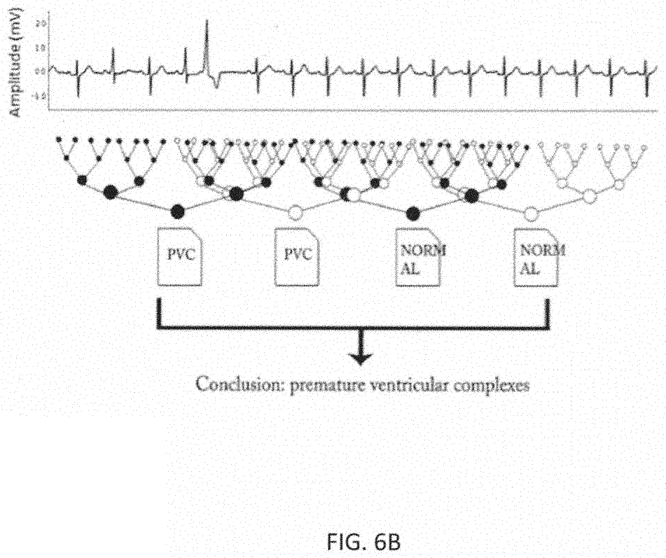

The classification neural network may be a convolutional neural network or a recurrent neural network. Referring now to FIG. 6A, a classification neural network in the form of a convolutional neural network is illustrated applied to an ECG signal. Most convolutional neural networks implement a few convolutional layers and then standard layers so as to provide a classification. The ECG signal is given as input to the network, which aggregates the information locally and then combines it layer by layer to produce a high-level multi-label classification of the ECG. For each label a score is provided. The labels of the convolutional neutral network shown in FIG. 6A include atrial fibrillation (AFIB), right bundle branch block (RBBB) and, and premature ventricular complex (PVC).

Referring now to FIG. 6B, a classification neural network in the form of a recurrent convolutional neural network is illustrated. Similar to FIG. 6A, the ECG signal is given as input to the network. A recurrent convolutional neural network refers to a particular convolutional neural network structure able to keep memory of the previous objects it has been applied to. A recurrent convolutional neural network is composed of two sub-networks: a convolutional neural network which extracts features and is computed at all time points of the cardiac signal, and a neural network on top of it which accumulates through time the outputs of the convolutional neural network in order to provide a refined output. In this manner, the convolutional neural network acts as a pattern detector whose output will be accumulated in time by the recurrent neural network.

As is shown in FIG. 6B, the output of the convolutional neural network identified four labels at various time points including premature ventricular complex (PVC) and Normal. Those labels were then applied to the second neural network which produced the refined output “premature ventricular complex.” In this example, the network correctly recognized a premature ventricular complex (PVC, the fifth and largest beat) in the first part of the signal while the second part of the signal is considered normal. As the cardiac signal includes abnormality, it cannot therefore be considered as normal, and the accumulated output is therefore PVC.

The first neural network (i.e., delineation neural network) and the second neural network (i.e., classification neural network) must be trained to achieve the behavior and functionality described herein. In both the delineation and the classification embodiments, the networks may be expressed using open software such as, for example, Tensorflow, Theano, Caffe or Torch. These tools provide functions for computing the output(s) of the networks and for updating their parameters through gradient descent.

Training the neural networks involves applying numerous datasets containing cardiac signals and known outputs to the neural networks. A database of the datasets containing cardiac signals collected across a plurality of patients using the systems and methods described herein may be stored on server 15 and/or drive 16 (e.g., in the cloud). The datasets in the database may be used by server 15 to analyze new cardiac signals inputted into the system for processing. In a preferred embodiment, any cardiac signal applied to the trained neural network will have the same sampling rate and/or frequency as the cardiac signals in the datasets used to train the neural network. For example, training of the classification neural network begins with a dataset containing cardiac signals and their known delineation. As explained above, the cardiac signal is expressed as a matrix of size m×n at a predefined frequency. For example, the network may be trained at 250 Hz, 500 Hz or 1000 Hz, though any frequency could be used. The delineation is then expressed in the form of a Matrix Y of size p×n where p is the number of types of waves. Each wave is expressed with their start and end points such as, for example: (P, 1.2 s, 1.3 s), (QRS 1.4 s 1.7 s), (T, 1.7 s, 2.1 s), (P, 2.2 s, 2.3 s). In this example, the first row of Matrix Y corresponds to P-waves, and will have a value of 1 at times 1.2 s and 1.3 s, and as well as 2.2 s and 2.4 s, and 0 otherwise. The second row of Matrix Y corresponds to QRS complexes and will have a value of 1 at times 1.4 s and 1.7 s, and otherwise 0. Finally, the third row of Matrix Y corresponds to T-waves and will have a value of 1 at times 2.2 s and 2.3 s, and otherwise 0. The parameters of the network may then be modified so as to decrease a cost function comparing the known delineation and the output of the network. A cross-entropy error function is used so as to allow for multi-labeling (i.e., allowing for multiple waves at a given instant). This minimization can be done though a gradient step, repeating the foregoing steps at least once for each cardiac signal of the dataset. It is understood that a similar approach may be used to train the delineation neural network (i.e., second neural network).

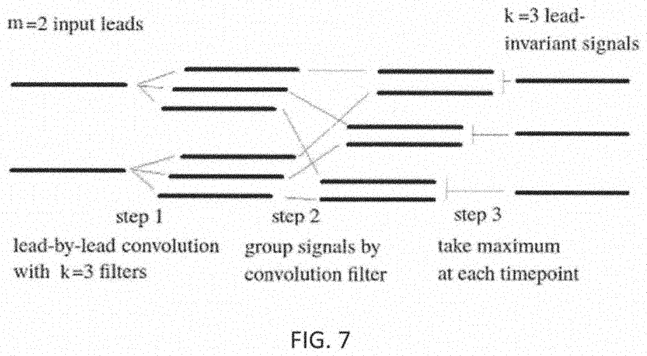

It is further understood that ECG platform 37 may cause neural networks described herein to process cardiac signals having a differing number of leads in entry. For example, the neural network may include a sequence of layers at the beginning of the network so as to obtain a network which is independent of the number of input leads and can therefore process cardiac signals with any number of leads m. For example, FIG. 7 illustrates two input leads (m=2) and three output signals (k=3). However, the same structure can process any number of input leads m and will still provide the same number of output signals, which can be fed to the rest of the network for which a fixed number of input signals is required. For this reason, the number of input leads may vary and need not be fixed.

As is shown in FIG. 7, to obtain k signals from an m input leads, the leads may be convoluted using a lead-by-lead convolution with k filters. The signal may then be grouped by a convolution filter in order to obtain k groups of m leads and a mathematical function is finally applied to each group to obtain k leads. The mathematical function may be the maximum at each time point or may be any other function known to one skilled in the art.

Referring again to FIG. 4, at step 61, ECG platform 37 may cause labels for each time window (i.e., labels) to be aggregated by post-processor 43 to generate processed labels 60. The labels may be derived from global measurements based on delineation. For example, the label corresponding to first degree atrioventricular block may be derived from a PR interval longer than 200 ms. As explained above, the PR interval is a global measurement based on the delineation. Post-processor 43 may also aggregate the delineation-based labels with the classification labels corresponding to the same time points.

Post-processor 43 may also filter the labels to remove redundant labels, assemble labels according to a known hierarchy of labels, or ignore labels that are known to be of lesser importance according to a hierarchy or weighted values. Post-processor 43 may also aggregate the labels through time so as to compute the start (onset) and end (offset) times of each abnormality. It is understood that post-processor 43 may be a standalone component or may be a subcomponent of classifier 41.

As is shown in FIG. 4, the information generated on back end 46 by ECG platform 37 in steps 54, 56, 58 and 61, and optionally, 63, may be communicated by ECG platform 37 to ECG application 29 on front end 45. ECG application 29 may cause the foregoing information to be displayed, at step 65, on display 17 of system device 14. The information generated on back end 46 may be automatically transmitted by ECG platform 37 or ECG platform 37 may cause the information to be stored on server 15 until requested by ECG application 29. Upon generating the data, ECG platform 37 may transmit a message to ECG application 29, informing ECG application 29 that the data is available from ECG platform 37.

ECG application 29 may receive data (e.g., raw ECG data, pre-processed ECG data, wave information, labels and any other data generated during steps 54, 56, 58, 61, and/or 63) and cause system device 14 to display as described in U.S. Patent Pub. No. 2020/0022604, the entire contents of which are incorporated herein by reference. Specifically, the '604 publication explains that the ECG signal, features of the ECG signal, and/or descriptors of the ECG signal may be displayed in a multiple field display in an interactive manner.

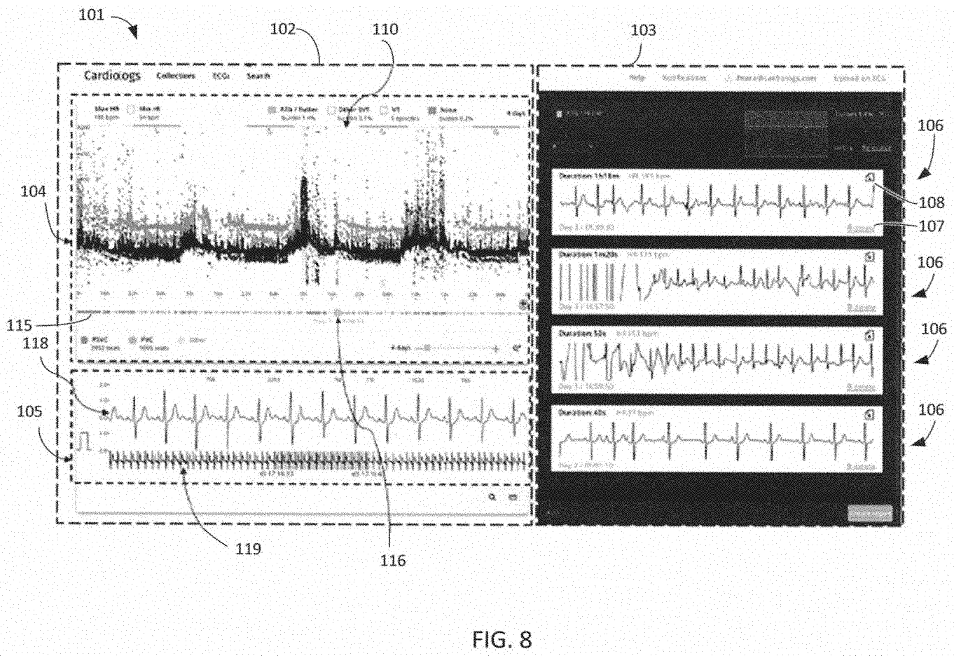

Referring now to FIG. 8, an exemplary display, interactive display 101, is illustrated. Interactive display 101 includes first side 102 and second side 103. First side 102 further includes second graphic window 105 and first graphic window 104, having plot 110 which includes data corresponding to the ECG signal. First graphic window 104 includes plot 110 providing a global view of an ECG signal.

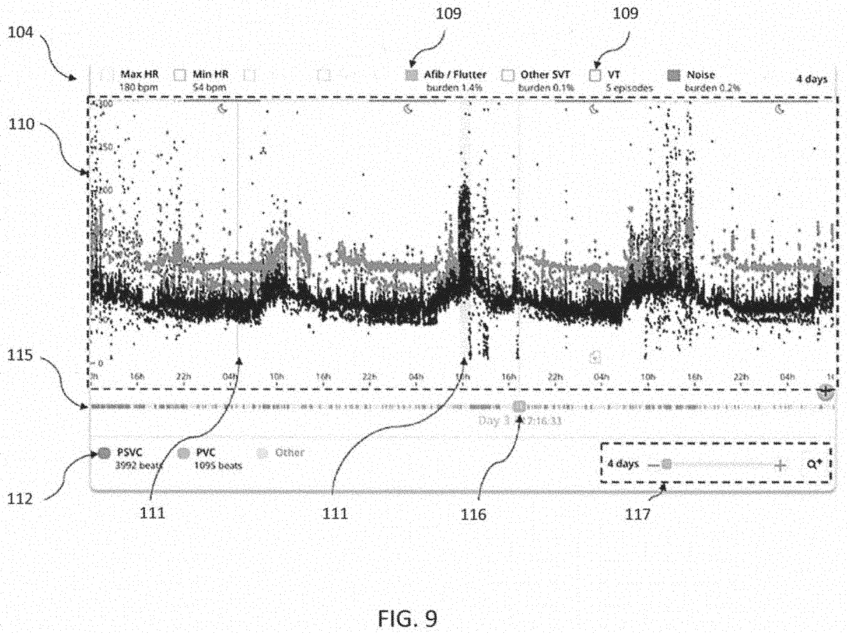

Referring now to FIG. 9, a zoomed-in version of first graphic window 104 is illustrated. In this exemplary display, plot 110 is an heart rate density plot (HRDP) which represents R-R intervals (interval between two QRS waves) through time. As shown in FIG. 9, the upper region of first graphic window 104 comprises multiple label buttons 109. Each label button 109 has, displayed in its proximity, text describing the label to which it is associated. Each label button 109 is associated with a color so that, when label button 109 is selected by the user, graphic portion 111 is displayed on the plot 110 to visually indicate the presence the episodes and/or events corresponding to the label associate with label button 109. This provides visual references for the user permitting easy identification of a specific category of events and/or episodes along the cardiac signal. In the exemplary display illustrated in FIG. 9, secondary labels 112 are included. In this exemplary display, secondary labels 112 include beat label PVC (premature ventricular complex) and PSVC (premature supraventricular complex), though it is understood that other secondary labels may be included. The points in the plot 110 associated with the label PVC and PSVC are colored, as shown in FIG. 9 by the presence of points of color different from black.

First graphic window 104 further comprises, parallel to the time axis of the plot 110, temporal bar 115. Temporal bar 115 provides a linear representation of the total ECG acquisition time wherein the time periods associated to episodes or events are represented as colored segments. As is shown in FIG. 9, the darker grey zones on temporal bar 115 correspond to time periods of noisy signal (e.g., when the signal is too artifacted and the analysis algorithm cannot propose a delineation and proper detection). First graphic window 104 further comprises interactive cursor 116. A user using ECG application 29 may move interactive cursor 116 along temporal bar 115 to allow a navigation of the plot 110 along the total ECG acquisition time. In the right bottom corner of first graphic window 104, first graphic window 104 comprises second interactive means 117 configured to cause plot 110 to zoom in and out.

Referring again to FIG. 8, second side 103 includes multiple episode plots 106. Each episode plot 106 displays at least one segment of the ECG strip corresponding to a detected episode and may include text regarding the duration (e.g., “Duration: 1 h 38 m”) and/or the starting time of the episode (e.g., “Day 3/09:39:30”). Each episode plot 106 includes third interactive icon 108 to select the corresponding episode plot for inclusion in a report. Each episode plot 106 further includes fourth interactive icon 107 which permits the user to remove the respective ECG plot from interactive display 101. Second side 103 may further include text describing one or more of episode plots 106.

Interactive display 101 further includes graphic window 105 including ECG strip 118 in a second time window starting at the time point selected by the cursor 116. Second graphic window 105 further includes ECG strip 119 in a third time window which is larger than the second time window which is inclusive of the second time window. The third time window includes a shaded portion which corresponds to the second time window.

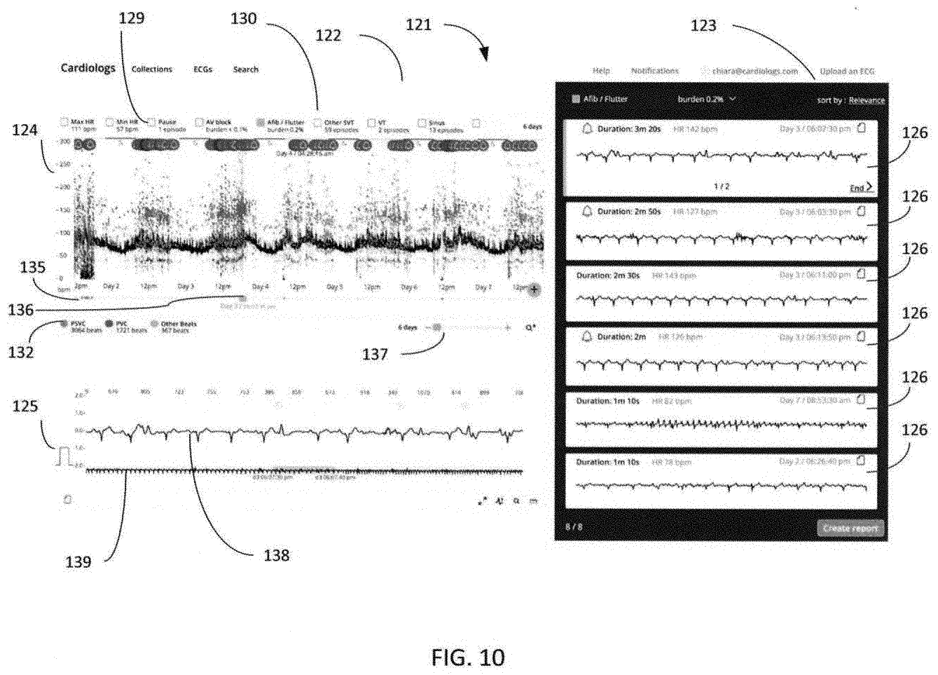

Referring now to FIG. 10, a similar display, interactive display 121, is illustrated. Interactive display 121 includes first side 122 and second side 123. First side 122 further includes first graphic window 124 and second graphic window 125. Second side 113 has the same functionality as second side 103 described above, and includes episode plots 126 similar to episode plots 106. Further, second graphic window 125 has the same functionality as second graphic window 105, and includes ECG strip 138 and ECG strip 139 similar to ECG strip 118 and ECG strip 119.

First graphic window 124 is similar to first graphic window 104 except for plot 130. Like first graphic window 104, first graphic window 124 includes multiple label buttons 129 having the same functionality as multiple label buttons 109, secondary labels 132 having the same functionality as secondary labels 112, temporal bar 135 and curser 136 having the same functionality as temporal bar 115 and cursor 116, and second interactive means 137 having the same functionality as second interactive means 117. Unlike plot 110, plot 130 is a heart rate density plot which is the projection onto a bivariate intensity plot of the histogram of the density of heart rates as a function of time.

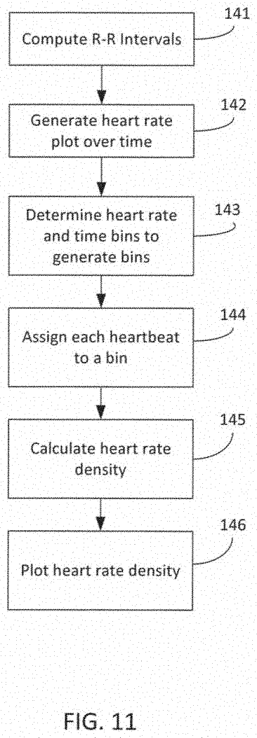

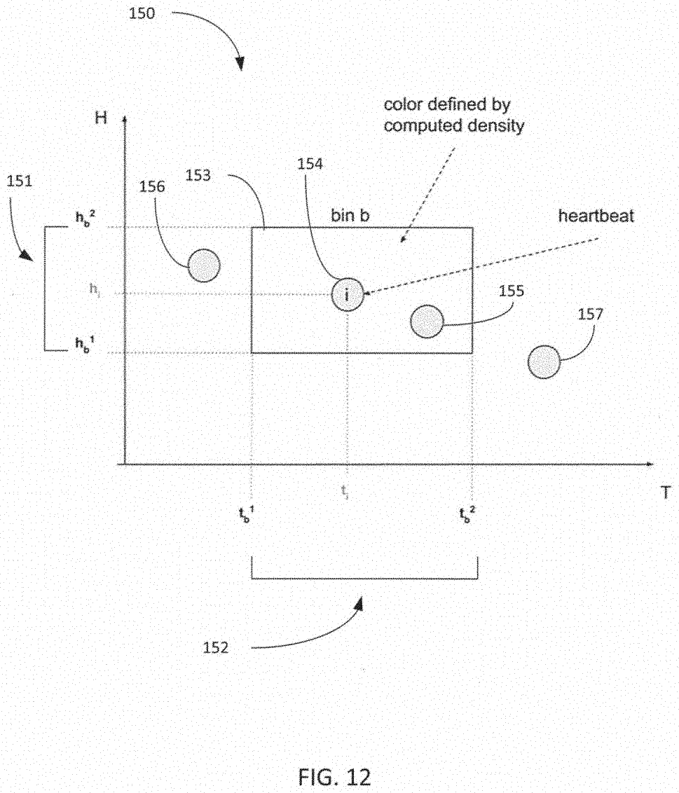

Referring now to FIG. 11, steps for generating and plotting a heart rate density plot, such as plot 130, are provided. At step 141, ECG platform 37 computes R-R intervals in the cardiac signal (i.e., ECG data). For example, ECG platform 37 may apply the cardiac signal to the delineation neural network to determine the RR intervals, as described above. At step 142, ECG platform 37 may generate the heart rate plot over time. An exemplary heart rate plot, HRDP 150, is illustrated in FIG. 12.

As is shown in FIG. 12, time is projected along the x-axis and the heart rate (e.g., beats per minute) is projected along the y-axis. In one embodiment, both time and heart rate are scaled linearly. However, time and/or heart rate may be scaled logarithmically or using other well-known scales. For simplicity, only four heart beats are shown in FIG. 12.

Referring again to FIG. 11, at step 143, ECG platform 37 may cause the y-axis and the x-axis may be divided into elementary elements, referred to as HR bins and time bins respectively. For example, in FIG. 12, HR bin 151 and time bin 152 are illustrated. HR bin 151 is defined by a first and second heart rate value (e.g., h_{b}¹ and h_{b}²). Similarly, time bin 152 is defined by a first and second time value (e.g., t_{b}¹ and t_{b}²). The intersection of a HR bin and a time-bin will be referred to as a bin. In other words, a bin will be defined by a first and second heart rate value and a first and second time value. In FIG. 12, bin 153 is illustrated and defined by HR bin 151 and time bin 152.

Referring again to FIG. 11, at step 144, ECG platform 37 will cause each heartbeat to be assigned to a bin. Specifically, a heartbeat (e.g., QRS complex) that occurs during a time window of a given time bin is included in the computation of the column corresponding to that time bin. Further, a heart rate corresponding to that heartbeat determines which HR bin it belongs to in the column defined by the time bin. For example, in FIG. 12, heartbeat 154 and heartbeat 155 each have a corresponding time and heart rate value that fall within time bin 152 and HR bin 151, respectively. Conversely, heartbeat 156 and heartbeat 157 each have a time value that falls outside time bin 151 and thus neither are included in bin 153.

Referring again to FIG. 11, at step 145, ECG platform 47 will calculate the heart rate density for each time bin. For a given bin, the area defined by the respective time bin and heart rate bin will be represented according to the density of the heart beats comprised in the bin (i.e., number of heartbeats within the bin). Each bin may then be color coded according to the density. For example, each bin may have certain shades of colors or patterns, such as grey levels, for example. In the example in FIG. 12, bins may be represented as levels of grey that get darker as the density of heart rates increases. As is shown in FIG. 12, bin 153, which includes 2 heartbeats, may be represented by a darker shade of grey than a bin with only 1 heartbeat, but a lighter shade of grey than a bin having 3 or more heartbeats.

In a preferred embodiment, the density is calculated as a function of the number of R-waves in the bin divided by the heart rate of the HR bin (e.g. the mean of the minimum and maximum bounds of the time window). This preferred computation of density considers the time spent in a specific bin. For example, in a time bin of 3 minutes, if there occurs 100 beats at a heart rate of 50 bpm (beats per minute) in a first HR bin and 100 beats at 100 bpm in a second HR bin, there will be as many beats in each bin, but 2 minutes will be spent at 50 bpm and only one minute at 100 bpm. Therefore, this bin would have the same density representation if only the number of beats are considered. However, when considering the count of beats divided by the heart rate, the first bin corresponding to the heart rate bin of 50 bpm will be darker than the bin corresponding to the heart rate bin of 100 bpm, as dividing by the heart rate gives higher weight to lower heart rate values. The preferred embodiment therefore captures this temporal information better than only considering the count of beats.

Referring again to FIG. 11, at step 146, ECG platform 37 will plot the heart rate density for each bin. It is understood that capturing temporal information in the column (time bin), in addition to the temporal information naturally given as function of the x-axis, facilitates expression of the density in a manner superior to other forms of aggregated representations of the ECG signal, such as the HRDP plot in plot 110.

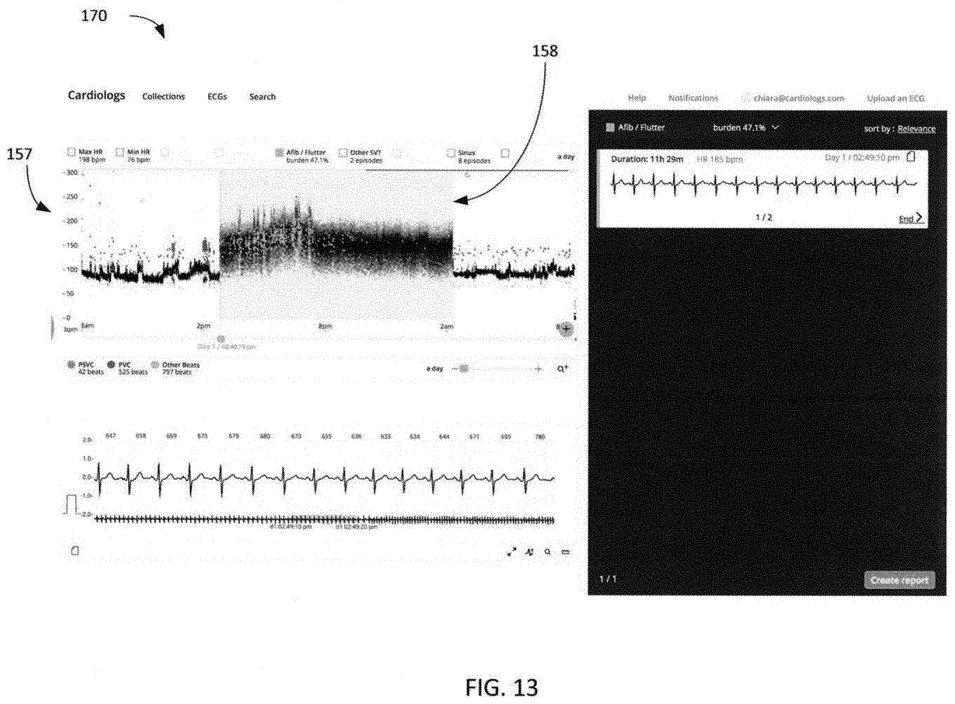

It is understood that the bounds of the x-axis of the HR density plot may be the beginning and end of the signal. However, in a preferred embodiment, the bounds of the x-axis may interactively vary with the action of zooming in and out performed by the user. The bounds of the y-axis remain fixed when performing this action. Referring again to FIG. 10, plot 130 includes interactive means 137 which may be used to zoom-in on the heart rate density plot. The zoom action may only change the size of the plot display. Alternatively, zooming in and out changes the size of the time window corresponding to a time-bin. With the zooming-in action, a bin represented with the same number of pixels covers a shorter time window. Zooming in therefore causes a new computation of the histogram with finer temporal divisions, and consequently, finer temporal information. This allows for a representation of the ECG signal that shows varying levels of aggregation of the information as a function of the time scale one chooses to display, in order for the histogram to remain both readable and informative at any level of zoom. Referring now to FIG. 13, an interactive display, interactive display 170, is illustrated which is similar to the interactive display in FIG. 10. Interactive display 170 has been zoomed-in resulting in plot 159 having zoomed in portion 158.

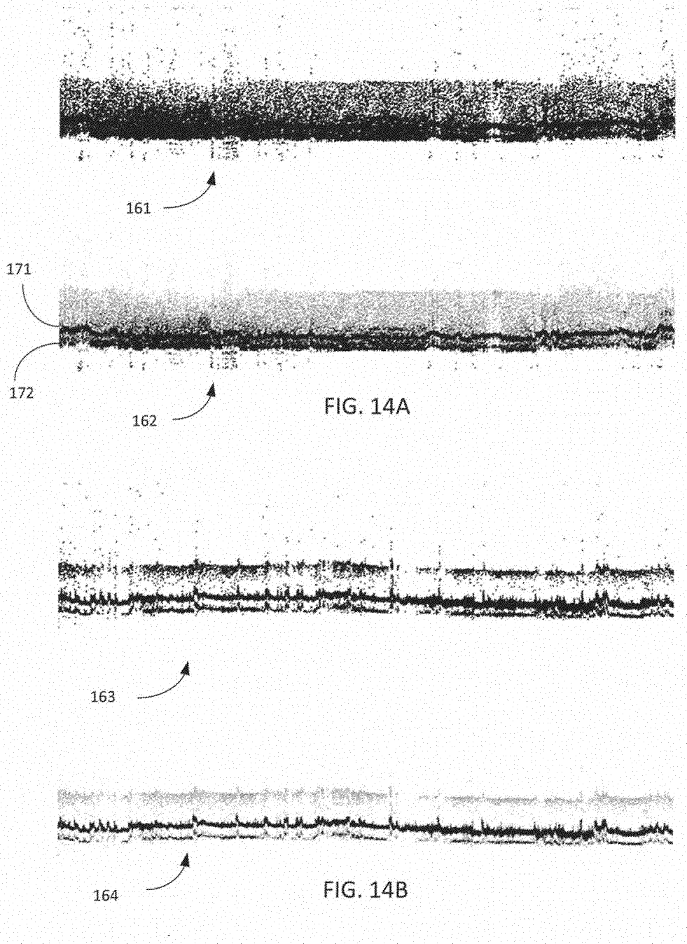

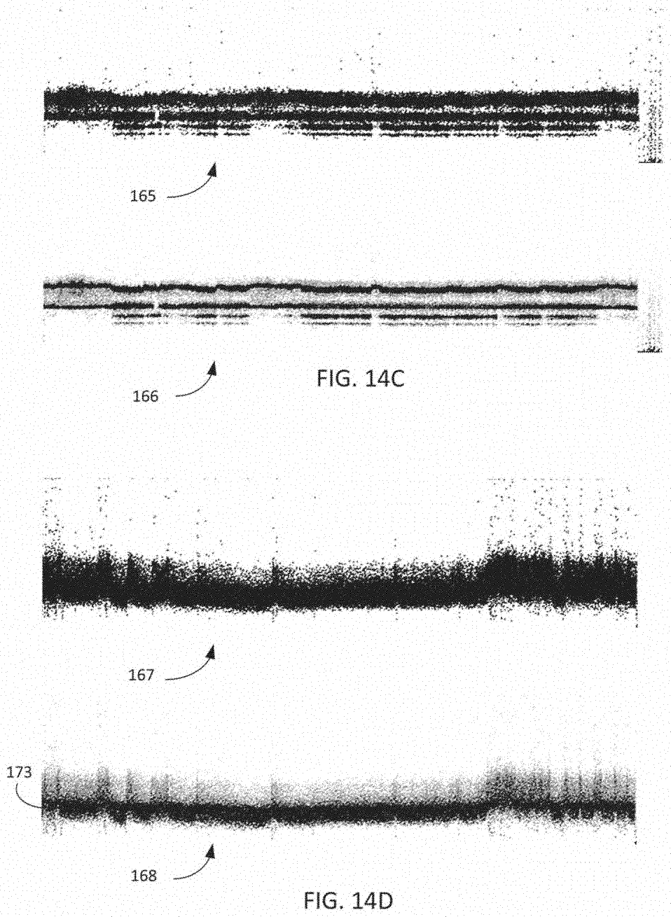

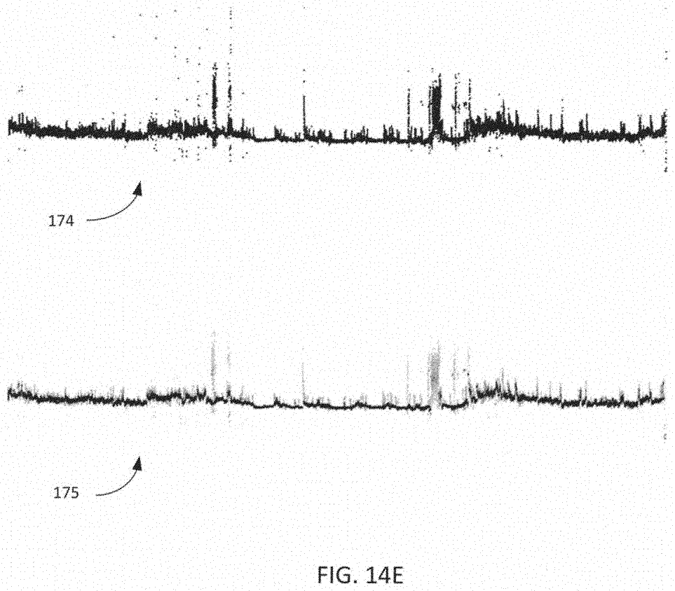

FIGS. 14A-E illustrate the superiority of the HRDP over the typical R-R plot. Referring now to FIG. 14A, a signal generated by a holter having a very high number of PVCs with varying coupling is illustrated as RR plot 161 and density plot 162. In density plot 162, the underlying rhythm is clearly visible as line 171. Further, the compensatory rest is illustrated as line 172 at the bottom. In R-R plot 161, this pattern is less clear. Referring now to FIG. 14B, a signal generated by a holter having less premature complexes than the one in FIG. 14A is illustrated as R-R plot 163 and density plot 164. The main rhythm is clearly illustrated in density plot 164 and is less clear in R-R plot 163. Referring now to FIG. 14C, a signal generated by a holter with vary conduction flutter is illustrated as R-R plot 165 and density plot 166. As is shown in FIG. 14C, the conduction flutter is more emphasized by the four clear black lines in density plot 166 than the four diffuse clouds that appear in the R-R plot 165. Referring now to FIG. 14D, a signal generated by a holter with permanent atrial fibrillation is illustrated as R-R plot 167 and density plot 168. As is shown in this figure, density plot 168 gives more precise information on the variations of the heart rate within the fibrillation. Specifically, darker lower half 173 shows that more time is spent at a low heart rate than at a high heat rate. Density plot 168 further illustrates spikes where the upper half becomes a bit darker corresponding to the heart rate increasing. These nuances are not visible in R-R plot 167. Referring now to FIG. 14E, a signal generated by a holter having paroxysmal atrial fibrillation and otherwise regular rhythm is illustrated as R-R plot 174 and density plot 175. The pattern of a regular rhythm is more visible in density plot 175 where a clear black line emerges. Also, the pattern of atrial fibrillation contrasts more in density plot 175 than R-R plot 174 as the color changes as well (density diminishes which makes the plot lighter).

Referring again to FIG. 4, at step 66, a user using ECG application 29 may interact with an interactive active display described above using input devices 25 to request a report and/or customize the report. A report typically includes portions of the cardiac signal and may involve information pertaining to abnormalities and/or episodes (e.g., episode plots) and/or other information generated during pre-processing (step 54), delineation (step 56), classification (step 58), clustering (step 63) and/or post-processing (step 61). A report may further include patient specific medical data such as the patient's name, age, health history, and/or other medical information. It is understood that any individually identifiable health information, and/or protected health information may be encrypted when communicated between ECG application 29 and ECG platform 37.

As explained above, interactive icons in interactive displays may be engaged to incorporate data and images displayed in a report. For example, third interactive icon 108 may be selected by a user using ECG application 29 to include the corresponding episode plot in a report. Accordingly, at step 66, the user may request a report and may select customized features such as certain data to be included in the report (e.g., abnormality data, episode data, episode plots, etc.).

At step 67, ECG application 29 may transmit the request for a report and selected customizable features (e.g., ECG data to be included in the report) to ECG platform 37 and ECG platform 37 may receive the request and information. ECG platform 37 may log the request and save the information received from ECG application 29. At step 68, ECG platform 37 may cause report generator 44 to generate a report 69 according to the information received from system ECG application 29.

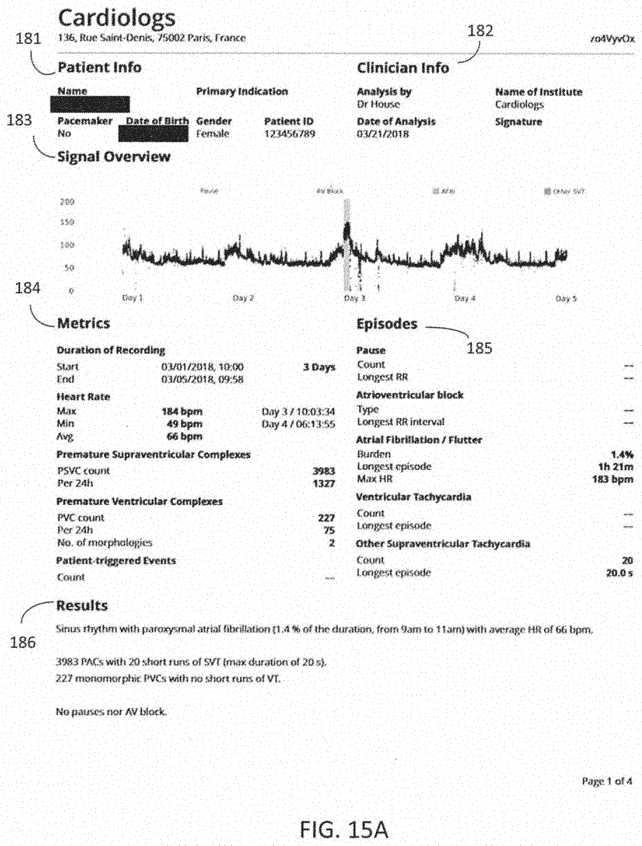

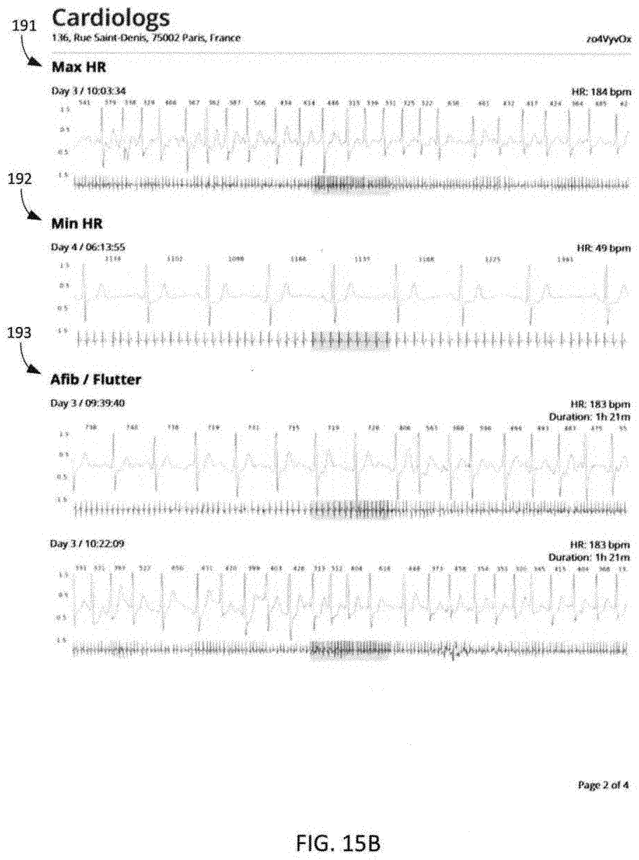

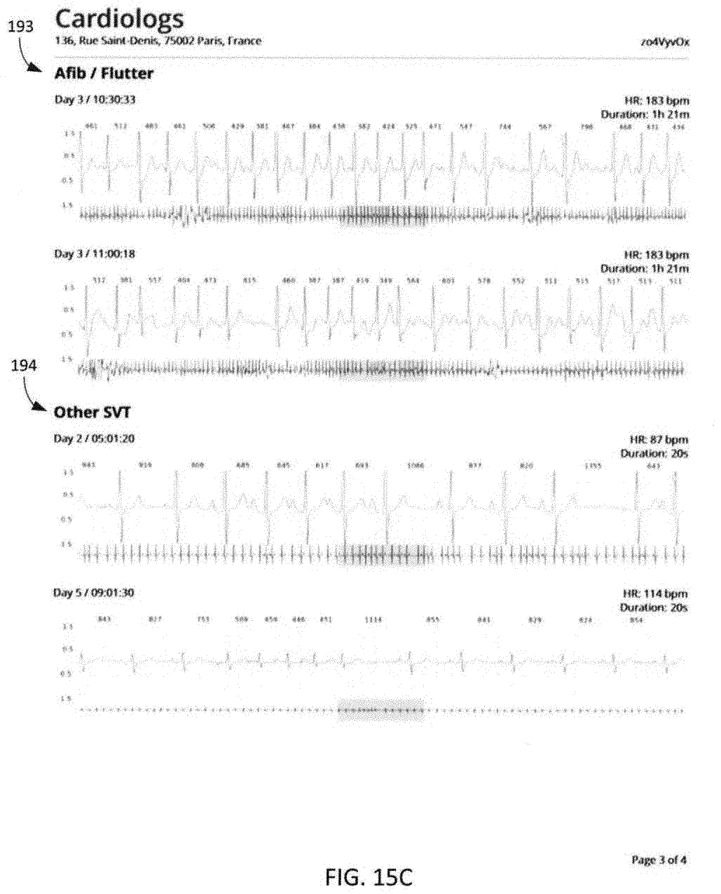

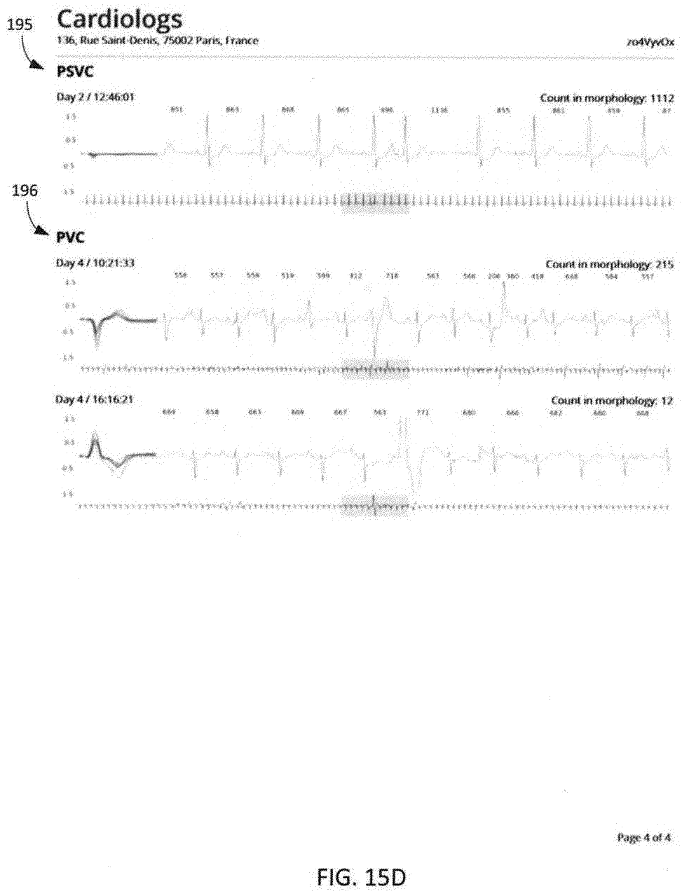

Referring now to FIGS. 15A-15D, an exemplary report generated at step 68 is illustrated. The first page of the exemplary report is illustrated in FIG. 15A. The first page may be presented in several sections such as first section 181, second section 182, third section 183, fourth section 184, fifth section 185, and sixth section 186. First section 181 may include patient specific information such as, for example, the patient's name, primary indication, whether the patient has a pace maker, the patients date of birth, gender and/or a patient ID. Second section 182 may include clinician information such as, for example, the overseeing physician, the name of the institute, the date of the analysis and/or a signature.

Third section 183 may include a plot of the ECG data. In FIG. 15A, section 183 includes a heart rate density plot similar to the one shown in FIG. 12. The window of time shown may be a default time or may be a user defined time window. Like the heart rate density plot in FIG. 12, a certain label may be selected to indicate the occurrence of an abnormality on the density plot. The time window is usually selected according to the relevant episodes and/or events. It is understood, however, that other plots may be included in the report such as an R-R plot.

Fourth section 184 may include metrics from the cardiac signal recording. For example, fourth section 184 may include the duration of the recording, the maximum, minimum and average heart rate, premature supraventricular complexes and any patient-triggered events, and/or any other metrics concerning the cardiac signal. Fifth section 185 may include information corresponding to any episodes detected. For example, fifth section 185 may include pause information (count and/or longest R-R interval), atrioventricular block information, atrial fibrillation/flutter information, ventricular tachycardia information, other supraventricular tachycardia information, and/or any other information concerning any episodes or abnormalities. Sixth section 186 may include results information such as, for example, a summary of the episodes and/or abnormalities, a diagnosis, and/or any other information analyzed, aggregated, computed, determined, identified, or otherwise detected from the cardiac signal. For example, sixth section 186 may identify a sinus rhythm with paroxysmal atrial fibrillation.

FIG. 15B-D illustrates the second, third and fourth pages of an exemplary report. As is shown in FIG. 15B-D, the report may further include ECG strips previously selected by the user, or selected under default settings. For example, a user may select Max HR strip 191, Min HR strip 192, Afib/Flutter strips 193, other SVT strips 194, PSVC strip 195, and PVC strip 196. Max HR strip 191 may be an ECG strip indicating the maximum heart rate during a given cardiac signal recording. Similarly, Min HR strip 191 may be an ECG strip indicating the minimum heart rate during a given cardiac signal recording. Afib/Flutter strips 193 may be ECG strips indicating each episode of atrial fibrillation/flutter. Other SVT strip 194 may be ECG strips indicating each episode of supraventricular tachycardia. PSVC strip 195 may be an ECG strip indicating an episode of premature supraventricular complex. PVC strip 197 may be ECG strips indicating episodes of premature ventricular complex. ECG strips may be displayed with the related relevant associated metrics and comments as added by the user. It is understood that the report shown in FIGS. 15A-B is merely exemplary and that the report generated at step 68 may have a different structure or configuration and/or may include different ECG and patient related information contemplated herein.

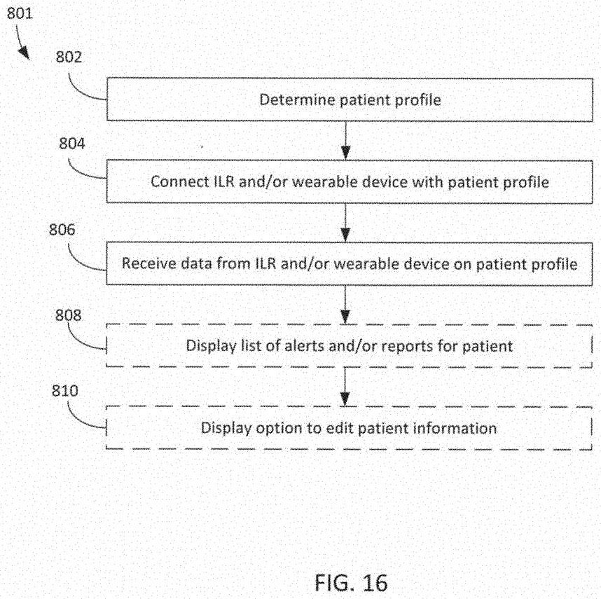

Referring now FIGS. 16-21C, the illustrated platform may be used by a user (e.g., physician, healthcare provider, technician), efficiently determine important data, to identify billable actions, tasks, and/or processes, and to label and/or classify certain actions, tasks, and/or processes for an electronic medical records (EMR) system. It is understood that the platform may be used for triaging data (e.g., classifying data as important or not), receiving and/or determining clinical decisions (e.g., writing a prescription, scheduling an appointment, etc.), determining certain billing information corresponding to the data (e.g., whether certain billing requirements for ILR monthly reports are satisfied). This may permit a physician, healthcare provider, and/or technician to bill for time related to an ILR and/or wearable device follow-up. Further the platform may generate a report that may be used to document a certain task and/or actions for the EMR. It is understood that the platform and the tasks and operations describe with respect to FIGS. 16-21C may be performed by ECG platform 37 illustrated in FIG. 3B.

Referring now to FIG. 16, an exemplary process for associating an implantable loop recorder (ILR) and/or wearable device (e.g., smart watch) with a patient profile on a platform is illustrated. For example, process 801 may be employed by a platform to determine ECG data from the ILR and/or wearable device, associate the ECG data from the ILR and/or wearable device with a patient profile, and determine alerts and/or reports corresponding to the data.

At block 802, a patient profile may be determined. For example, a user (e.g., physician, healthcare provide, and/or technician) may generate a profile for a particular patient. At block 804, a ILR and/or wearable device of a patient may be connected and/or associated with the patient profile such that data from the ILR and/or wearable device is periodically sent to and/or shared with the platform.

At block 806, the platform may receive data from the ILR and/or wearable device and may archive the data on a server and associate the data with the patient profile. For example, a server running the platform may receive data from the ILR and/or wearable device and may determine, based on a device identifier or a user identifier that the device is known and associated with a user profile and may archive that data in a manner that associates the data with the user profile.

At optional block 808 the platform may optionally display a list of the data, alerts and/reports based on the data. The platform may automatically generate alerts after processing the data using the techniques described herein (e.g., using delineation, classification, clustering, etc.). The platform may also automatically and/or at the direction of the user, generate reports corresponding to the data as described herein. At optional block 810, the platform may display an option to edit the patient information and/or any other information in the patient profile. For example, the user may alter the arrangement of the alerts and/or data displayed at optional block 808.

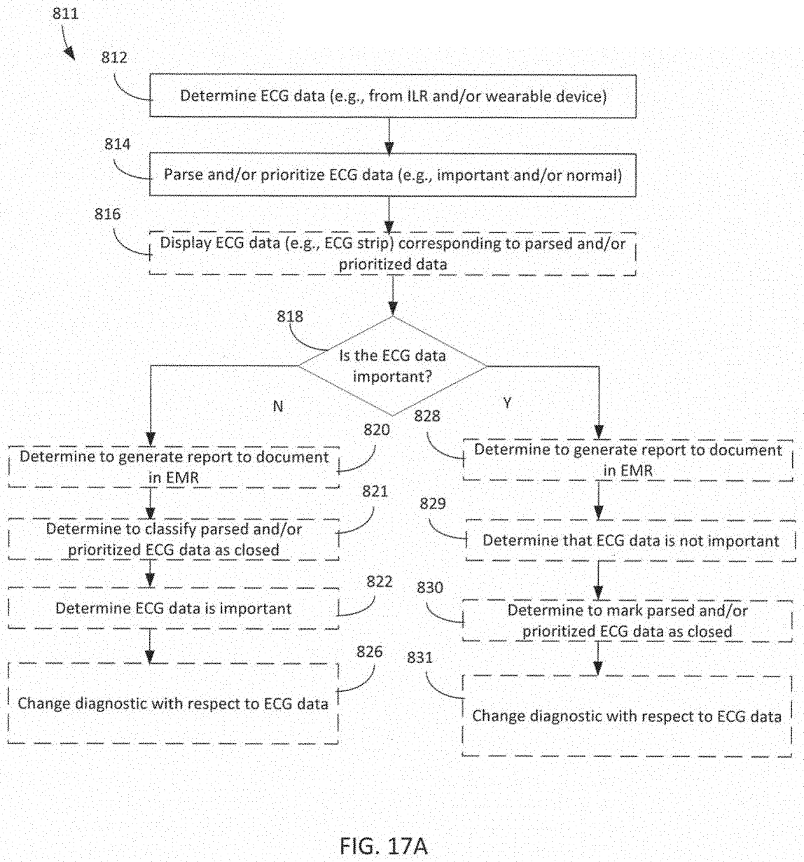

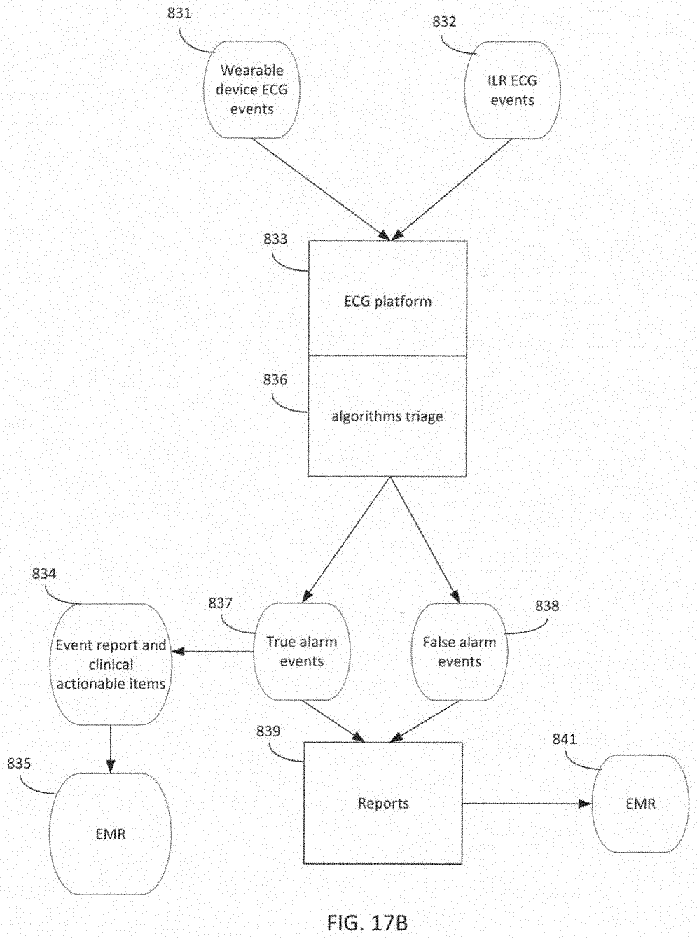

Referring now to FIG. 17A, an exemplary process for determining data from a loop recorder implantation (ILR) and/or wearable device (e.g., smart watch), determining if the data is important, and determining to take certain actions with respect to the data is illustrated. At block 812, the platform may determine ECG data (e.g., from a loop recorder implantation (ILR) and/or wearable device (e.g., smart watch). This may be the same step as step 806 of FIG. 16. At block 814, the data may be parsed and/or prioritized. For example strips of ECG may be determined and may be assigned a label as described herein (e.g., using delineation, classification, clustering, etc.). As shown in FIG. 17A, the ECG data may be determined to be either normal or important. The normal and/or important important label may be determined using the algorithms and techniques described herein and/or patient medical history and/or physician preference At optional block 816, the ECG data may be displayed based on the determination made at block 814. For example, ECG strips may be labeled as either important or normal. A user may elect to display the important and/or normal ECG strips.

At decision 818, if the ECG data is not important, at optional block 820, the platform (e.g., either automatically and/or at the direction of the user) may generate a report to document the important ECG data for EMR purposes. This may include generating a report as described herein. At optional block, the platform may determine to classify parsed and/or prioritized ECG data as closed. At optional block 822, the platform may further determine that the ECG data that was initially categorized as normal is important based on user feedback. For example, a user may view displayed ECG strips classified as normal and may instruct the platform that the ECG is important. At optional block 826, the user may change one or more diagnostics with respect to the ECG data.

If instead, at decision 818, the ECG data is important, the platform (e.g., either automatically and/or at the direction of the user) may generate a report to document the important ECG data for EMR purposes at optional block 828. This may include generating a report as described herein. At optional block 829, the platform may determine that the ECG data is not important (e.g., based on user feedback). At optional block 830, the platform may further determine to mark the parsed and/or prioritized ECG data and/or an event corresponding thereto as closed. For example, a user may view displayed ECG strips classified as important and may instruct the platform to mark the event and/or data as closed. At optional block 831, the user may change one or more diagnostics with respect to the ECG data.

Referring now to FIG. 17B, an exemplary data flow for determining ECG event data, determining alarms, and applying the data to EMR is illustrated. As shown in FIG. 17B, wearable device ECG events and/or ILR ECG events may be communicated to ECG platform 833, which may be the same as ECG processing system 10 and/or ECG platform 37. For example, ECG processing system 10 and/or ECG platform 37 may include algorithms triage module 836 which may determine whether ECG data and/or events are normal or important. As explained above with respect to FIG. 17A, an event may be normal even if there is noise. The ECG platform may process ECG events (e.g., ECG data) and classify it as important if it is abnormal (e.g., atrial fibrillation).

Based on the data received by ECG platform 836, true alarm events 837 and/or false alarm events 838 may be determined. For example ECG platform 836 may employ the techniques described herein (e.g., delineation, classification, clustering, etc.) to analyze wearable device ECG events 831 and/or ILR ECG events 832. True alarm events may correspond to the ECG platform correctly classifying the ECG event and/or data. False alarm events may correspond to the ECG platform incorrectly classifying the ECG event and/or data (e.g., based on user feedback). True alarm events and/or false alarm events may be used by reports module 839 to update EMR 841 and otherwise cause EMR 841 to incorporate this information.

The true alarm events may be used by the platform to generate item 834, which may include an event report and/or clinical action items. For example, ECG platform 833 may generate a report for important ECG events. The report may include ECG strips. Additionally, or alternatively, ECG platform may determine clinical actionable items and/or recommendations (e.g., in the form of a message and/or alarm). The information in item 834 may be used by and/or incorporated in EMR 835.

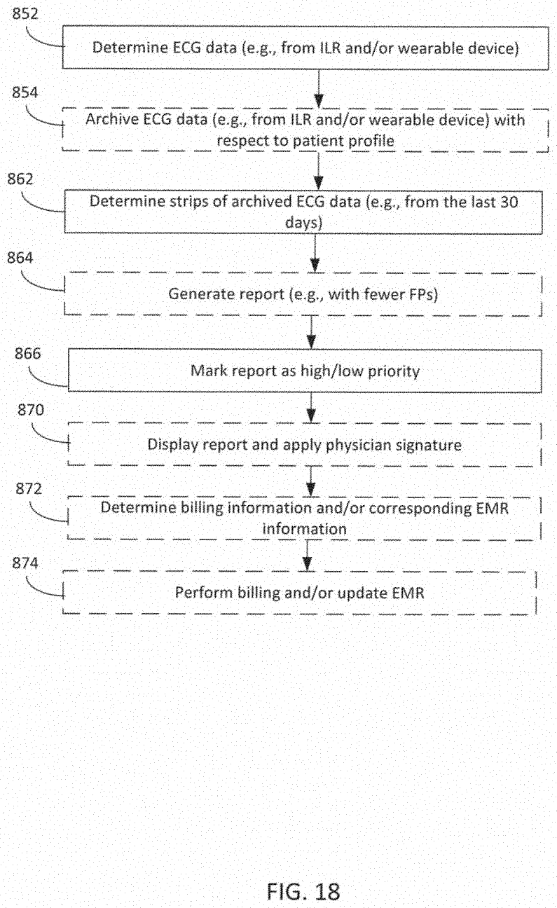

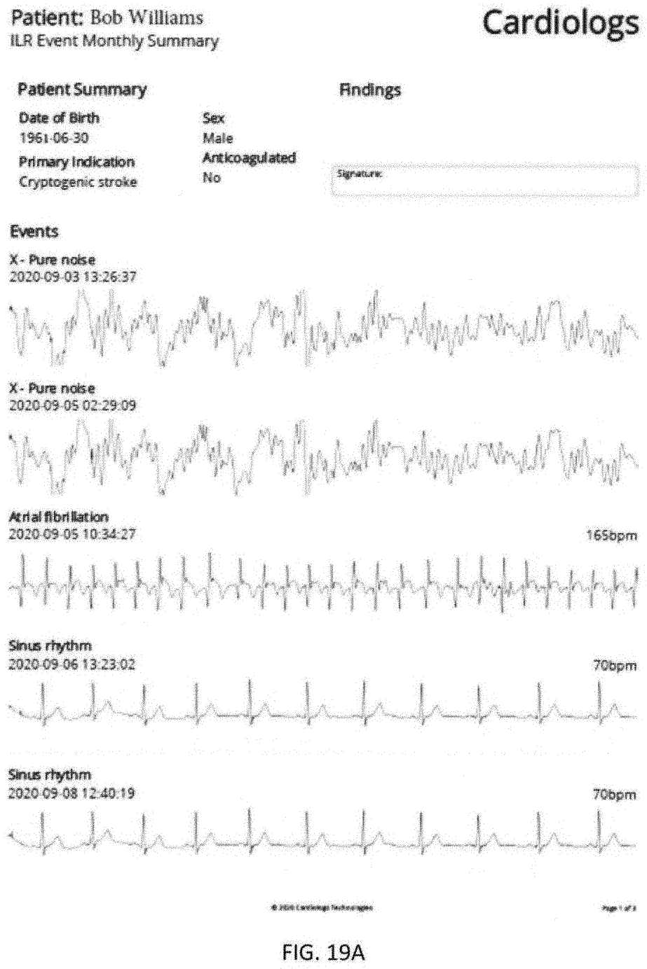

Referring now to FIG. 18, an exemplary process for determining data from a loop recorder implantation (ILR) and/or wearable device (e.g., smart watch), determining a priority and applying a physician signature to a report. At block 852, ECG data may be determined (e.g., from ILR and/or a wearable device). This step may be the same as step 812 of FIG. 17A. At optional block 854, the ECG data (e.g., from the ILR and/or wearable device) may be archived and/or otherwise saved (e.g., on a server). The data maybe be associated with a patient profile. At block 862 strips of archived ECG data may be determined. For example, a number of strips over a period of time may be determined (e.g., 30 strips over 30 days). At optional block 864, a report may be generated based on the ECG data (e.g., with fewer FPs).

At block 866, a report generated (e.g., at block 864) may be classified as a high or low priority. The priority designation may be assigned based on the presence of important information. The reports may include billing information and/or requirements, all ECG strips for a given period of time, and/or certain trends (e.g., HR trends). Alternatively, or additionally, a physician may review the report and determine the priority designation (e.g., high or low). At optional block 870, a report may be displayed and the platform may receive instructions to affix a signature to the report. At optional block 872, the platform may determine billing information and/or corresponding EMR information based on the report and/or data in the report. At optional block 874, billing may be performed based on information in the report and/or EMR may be updated such that relevant information from the report is applied to or otherwise incorporated into the EMR.

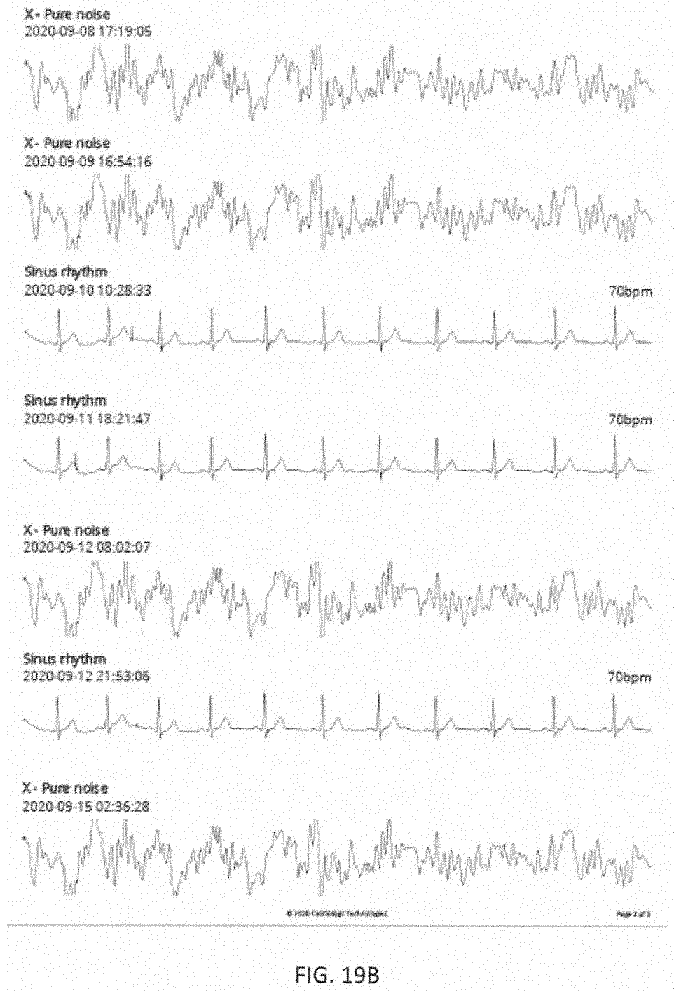

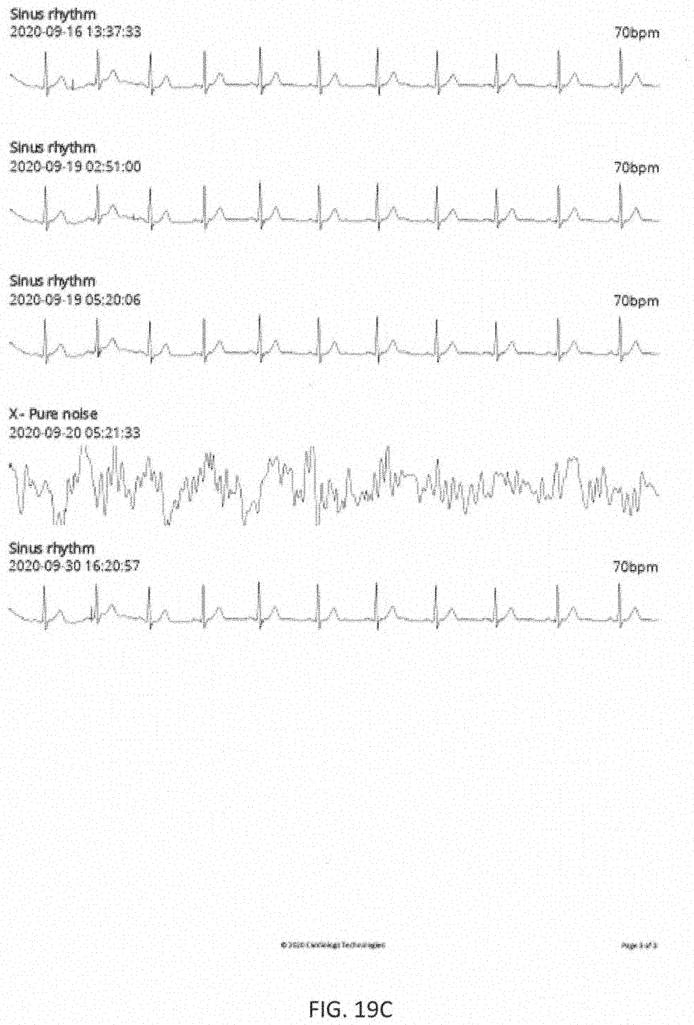

Referring now to FIGS. 19A-C an exemplary ILR/wearable device event report is illustrated. As shown in FIGS. 19A-C, the report may include patient information and ECG strips for various events (E.g., atrial fibrillation, sinus rhythm, etc.). While the report illustrates a month summary, it is understood that any other time frame may be included in a report. It is understood that the physician may add comments and/or sign the report.

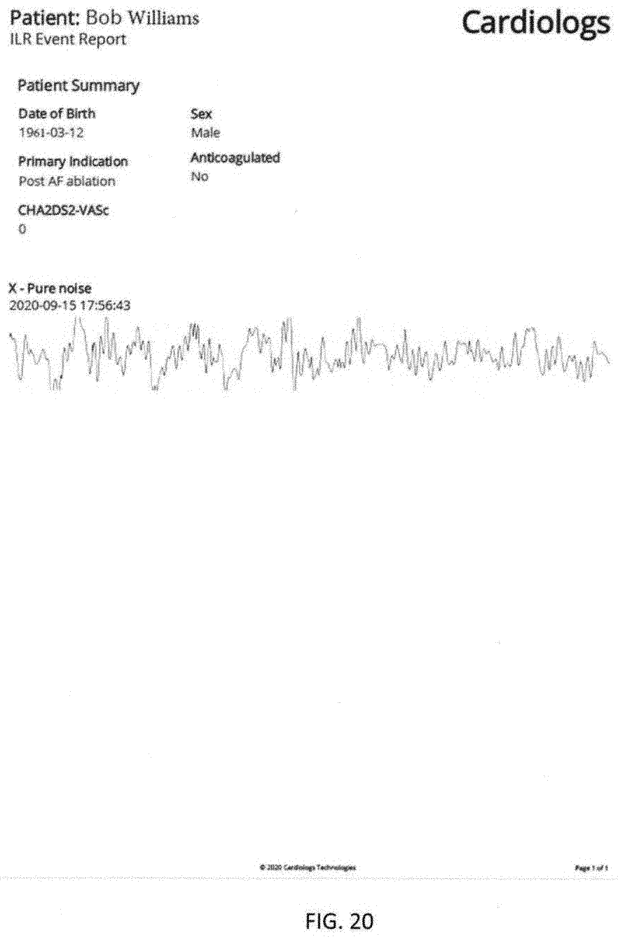

Referring now to FIG. 20, an exemplary ILR/wearable device event report is illustrated. The ILR event report may include information such as patient summary (e.g., including a primary indication) and/or an event ECG strip.

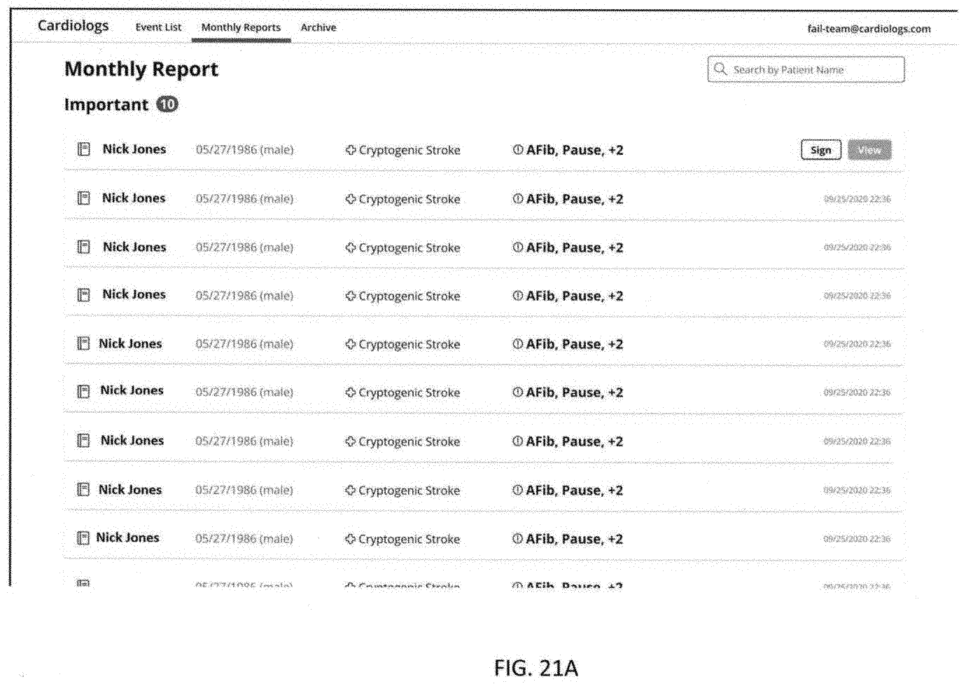

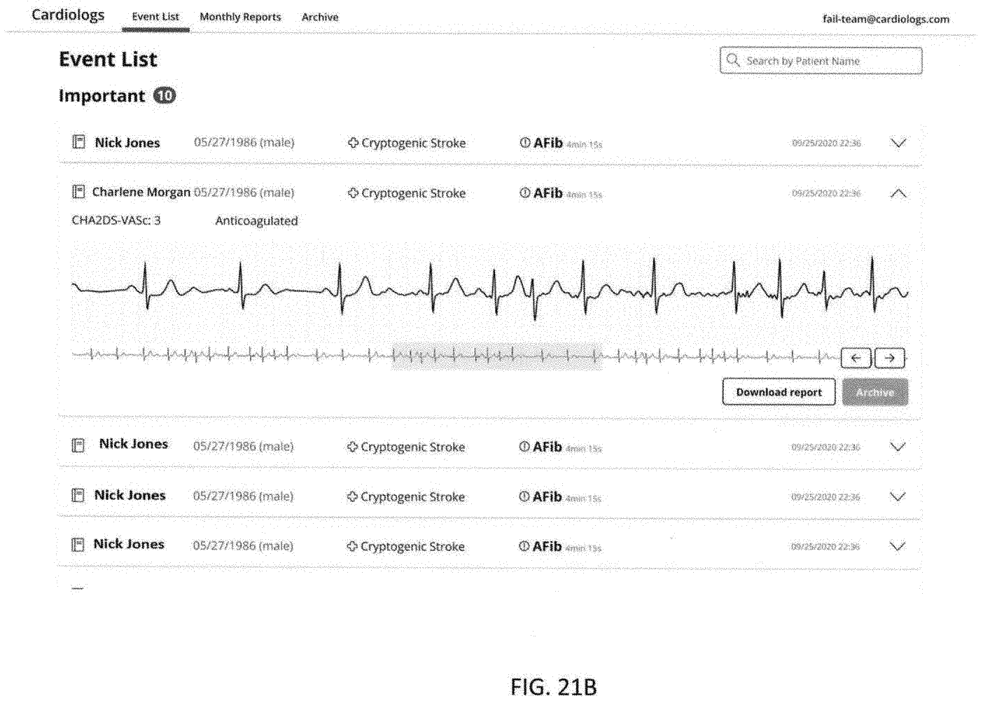

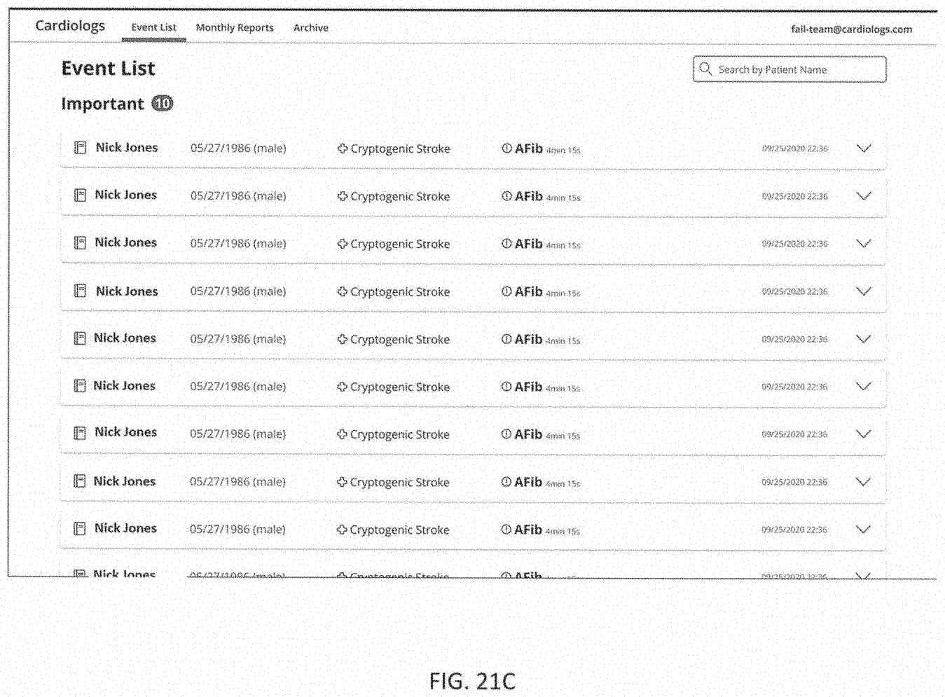

Referring now to FIGS. 21A-C, exemplary monthly report and event list user interfaces are illustrated. As shown in FIG. 21A, a monthly report may include a list of reports that have been identified as important and/or normal. Each event may include the patient's name, birthday, indication, event classification and/or description, and/or any other information (e.g., event data). Each report may be viewed and/or signed by a user, as described above with respect to FIG. 18. As shown in FIG. 21B, the platform may display an event list for events that are classified as important and/or normal. Each event may include the patient's name, birthday, indication, event classification and/or description, and/or any other information. The invention list may include one or more ECG strips for viewing the event. Each event in the event list may include the option to download a report, archive, and/or change priority level. As shown in FIG. 21C, the event list may optionally only include the patient's name, birthday, indication, event classification and/or description to streamline viewing.

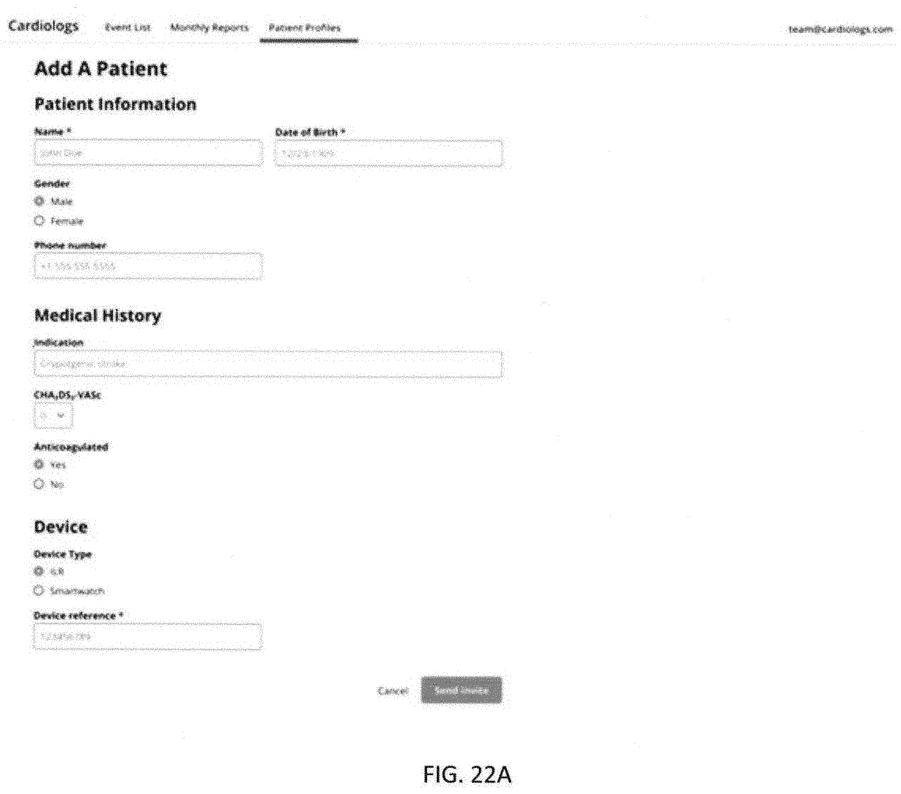

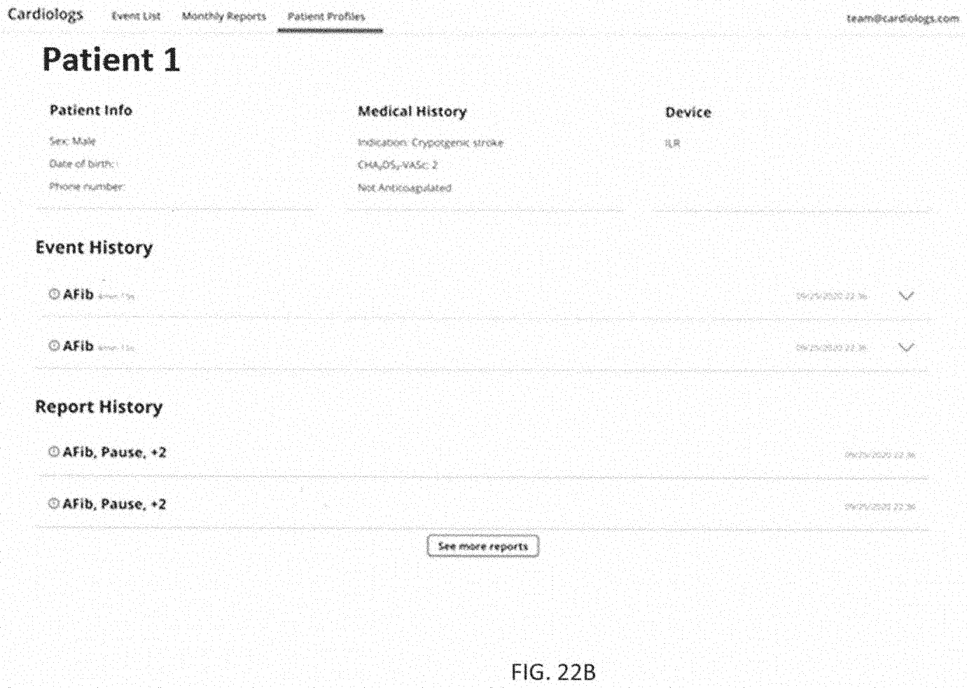

Referring now to FIGS. 22A-22B, exemplary user registration and profile interfaces are illustrated. As shown in FIG. 22A, an exemplary user registration interface may be used to add a patient and generate a user profile including the user name, date of birth, gender, contact information, medical history, device, and the like. As shown in FIG. 22B, an exemplary user profile may include patient information, medical history information, device information, event history, report history, and the like.

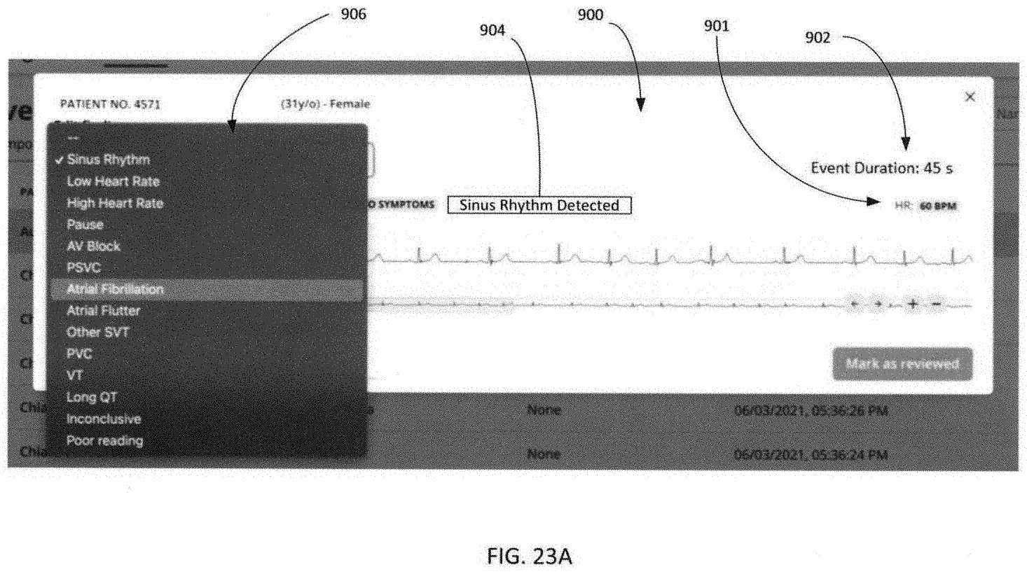

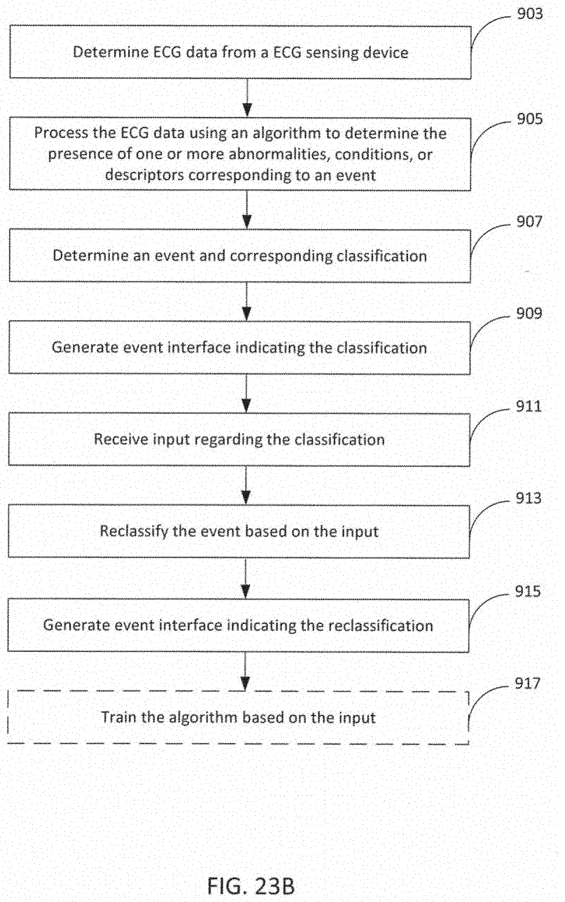

Referring now to FIGS. 23A-B, an exemplary event interface and process for reclassifying the event interface are illustrated. Referring now to FIG. 23A, event interface 900 is illustrated. Event interface 900 may display a portion of an ECG signal where an event was detected (e.g., using one or more approaches described herein). Event interface 900 may include heart rate indicator 901 which may display a detected or estimated heart rate corresponding to a point or interval of the ECG signal or alternatively an average, minimum, or maximum heart rate. Additionally, event interface 900 may include event duration 902, which may correspond to an event on-set and an event off-set. It is understood that any other relevant information (e.g., QTc) may displayed in event interface 900. Such information may be based on the delineation analysis described herein, for example.

FIG. 23A may further include classification box 904 and reclassification menu 906. Classification box 904 may display one or more classifications (e.g., conditions, abnormalities, descriptors, etc.) associated with the ECG signal. For example, classification box 904 may state “sinus rhythm detected.” Reclassification menu 906 may include a menu of selectable options for reclassifying the event detected in the ECG signal. For example, reclassification menu may include one or more of low heart rate, high heart rate, pause, AV block, PSVC, atrial fibrillation, atrial flutter, other SVT, PVC, VT, Long QT, or any other condition or abnormality. Reclassification menu 906 may further include additional classifications such as “inconclusive” and/or “poor reading.” By selecting an abnormality, condition or other information in reclassification menu 906, the event identified in event interface 900 may be reclassified. The reclassified event may be used to train the algorithms, neural network architectures, and models used to initially classify the event.

Referring now to FIG. 23B, an exemplary process for generating (e.g., by the ECG platform) an event interface including a classification of the event and reclassifying the event based on the event interface is illustrated. Some or all of the blocks of the process in FIG. 23B may be performed in a distributed manner across any number of devices (e.g., computing devices and/or servers). Some or all of the operations of the process in FIG. 23B may be optional and may be performed in a different order.

To initiate the process set forth in FIG. 23B, at step 903, ECG data from an ECG sensing device (e.g., ILR) is determined and/or obtained. At step 905, the ECG data may be processed using an algorithm to determine the presence of one more abnormalities, conditions, or descriptors corresponding to an event (e.g., cardiac event, ECG event, and/or any other physiological event). At step 907, one or more classifications corresponding to theevent may be determined using the algorithm. For example, the classification “sinus rhythm” may be determined based on the presence of one or more abnormalities, conditions, or descriptors.

At step 909, an event interface may be generated indicating (e.g., displaying) the classification and/or cardiac event determined at step 907. For example, the event interface may display “sinus rhythm” and may include a representation of the ECG signal corresponding to the event. At step 911, input regarding the classification may be received. For example, a system device (e.g., healthcare provider device) may present the event interface and the healthcare provider may send the ECG platform a message regarding the classification (e.g., regarding the accuracy of the classification).

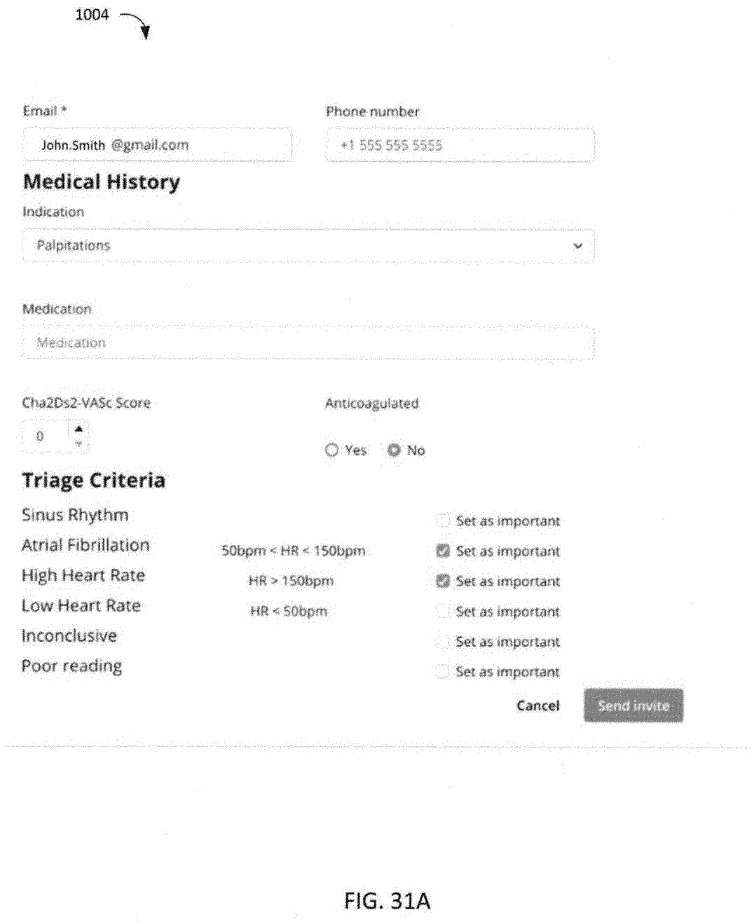

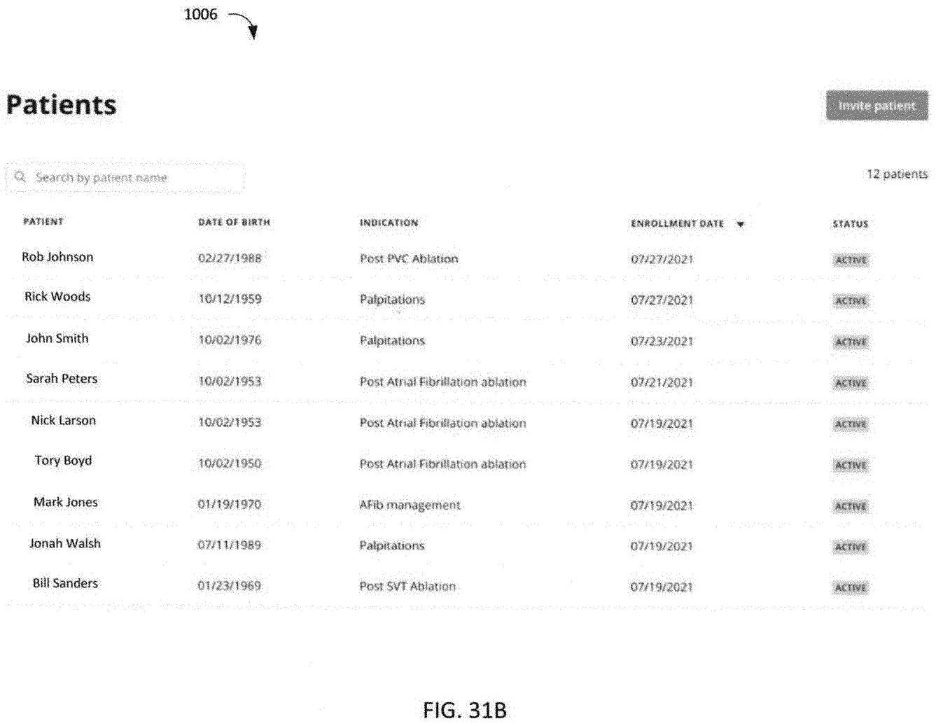

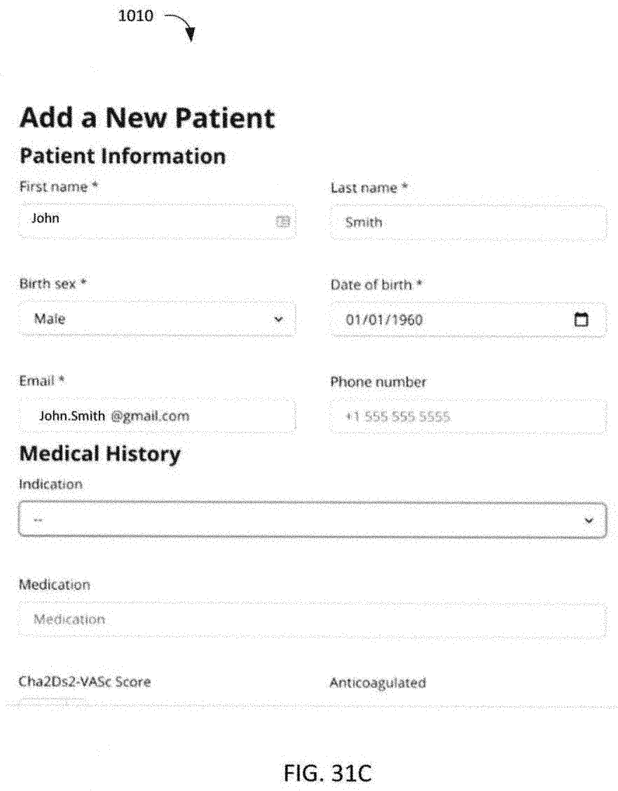

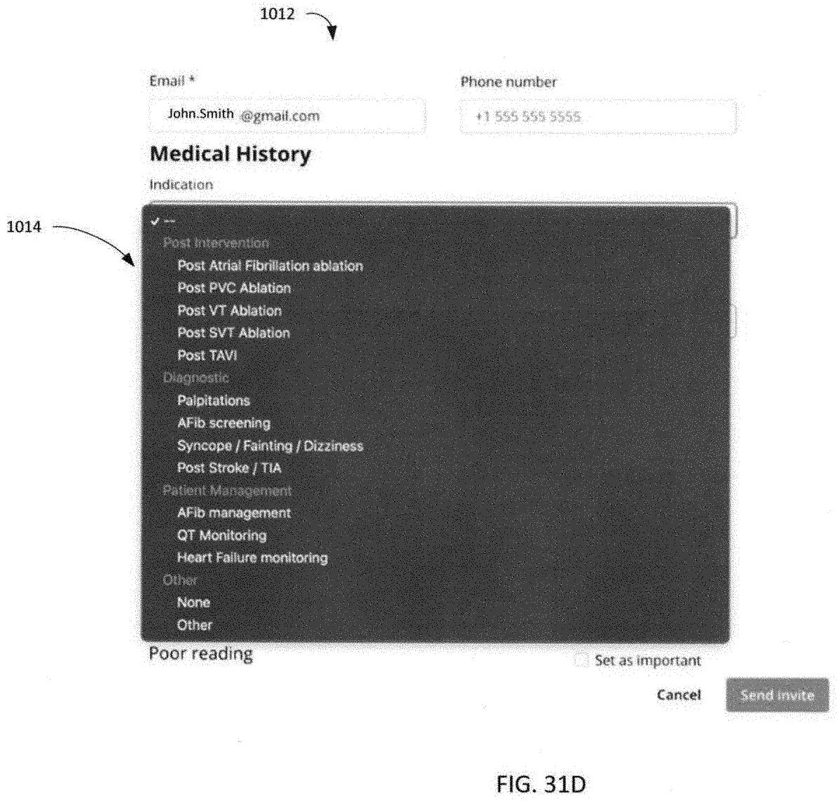

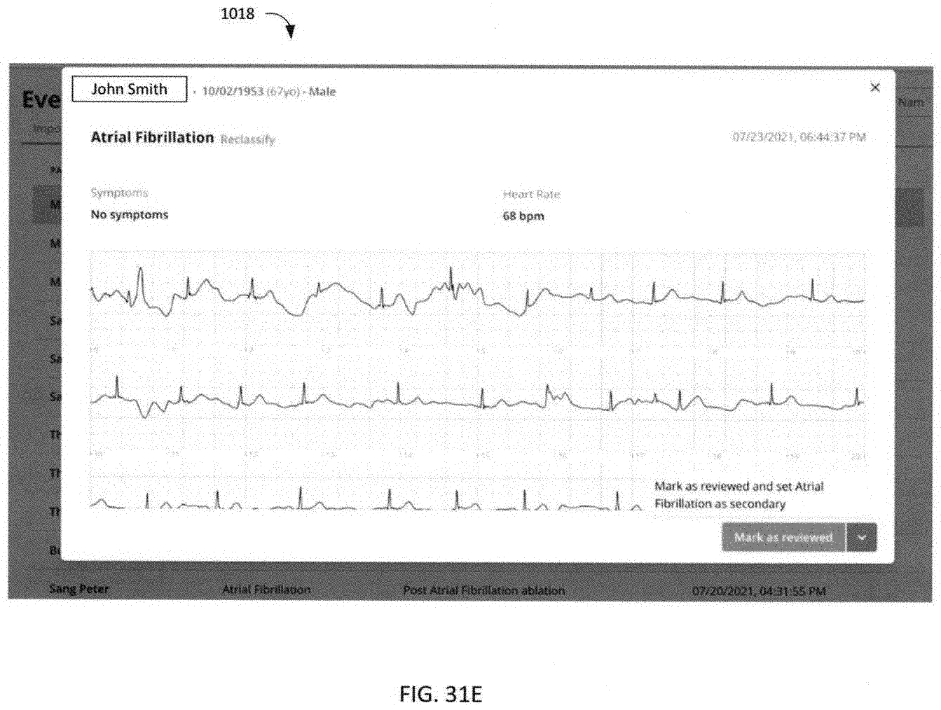

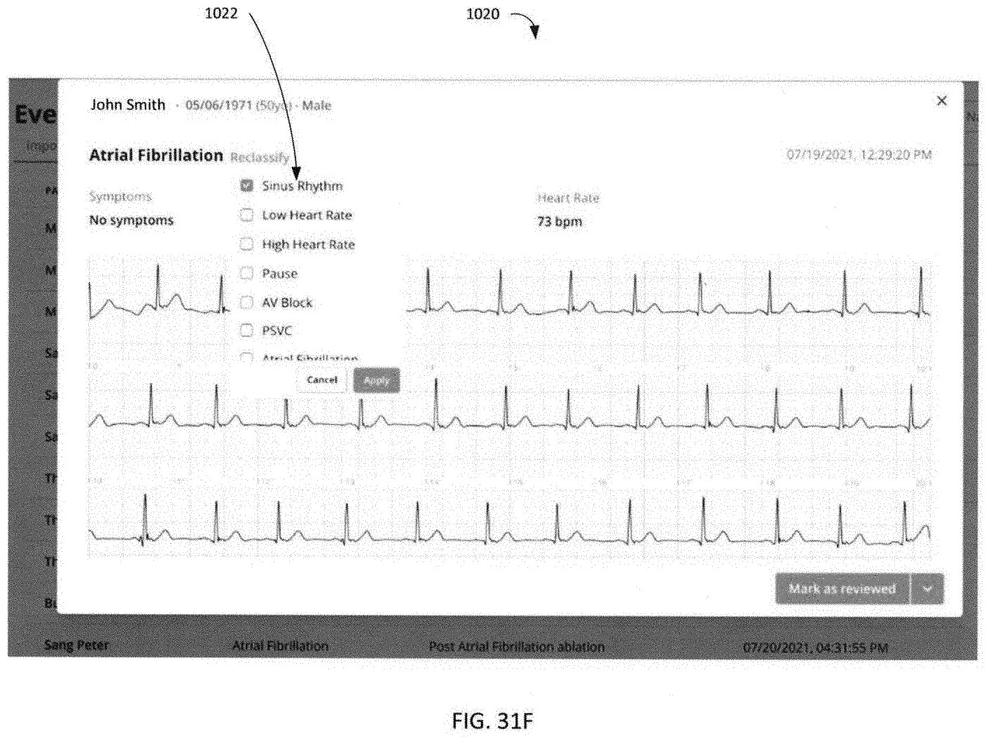

At step 913, the cardiac event may be reclassified based on the input received. For example, the input may indicate that the classification determined at step 907 was not accurate and may even identify a new classification. The new classification may be used to reclassify the event. At optional step 915, an event interface may be generated indicating the reclassification determined at step 913. At optional step 917, the algorithm used to process the ECG data at step 905 may be trained and/or otherwise modified based on the reclassification. Event interfaces and reclassification are described in greater detail below with respect to FIGS. 31E-31F.

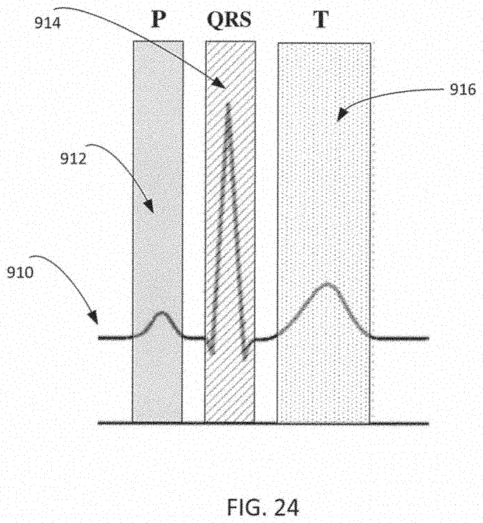

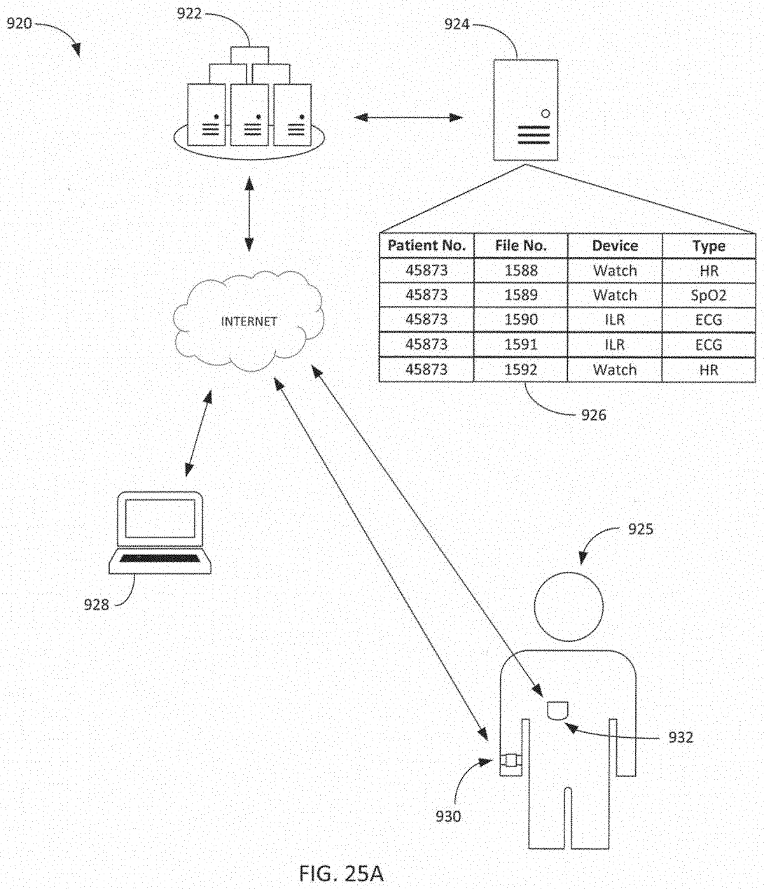

Referring now to FIG. 24, an exemplary ECG signal with color bands is illustrated. Specifically, ECG display 910 may be a portion of the ECG signal displayed in the event interface illustrated in FIG. 23A and/or any other presentation of an ECG signal and may include color indictors 912, 914, and 916. Color indicator 912 may be any color and/or pattern different from color indicators 914 and 916 and may indicate this portion of the ECG signal corresponds to a p-wave, for example. Color indicator 914 may be any color and/or pattern different from color indicators 912 and 916 and may indicate that this portion of the ECG signal corresponds to a QRS complex, for example. Color indicator 916 may be any color and/or pattern different from color indicators 912 and 914 and may indicate that this portion of the ECG signal corresponds to a t-wave, for example. It is understood that any color or pattern may be used to differentiate various portions of the ECG signal. It is further understood that color indicators may be used to indicate any portion and/or feature of an ECG signal (e.g., hidden p-wave, QT interval, ST segment, RR interval, TP segment, PR segment, and the like). The color indicators may be based on the delineation analysis and/or any other analysis described herein.

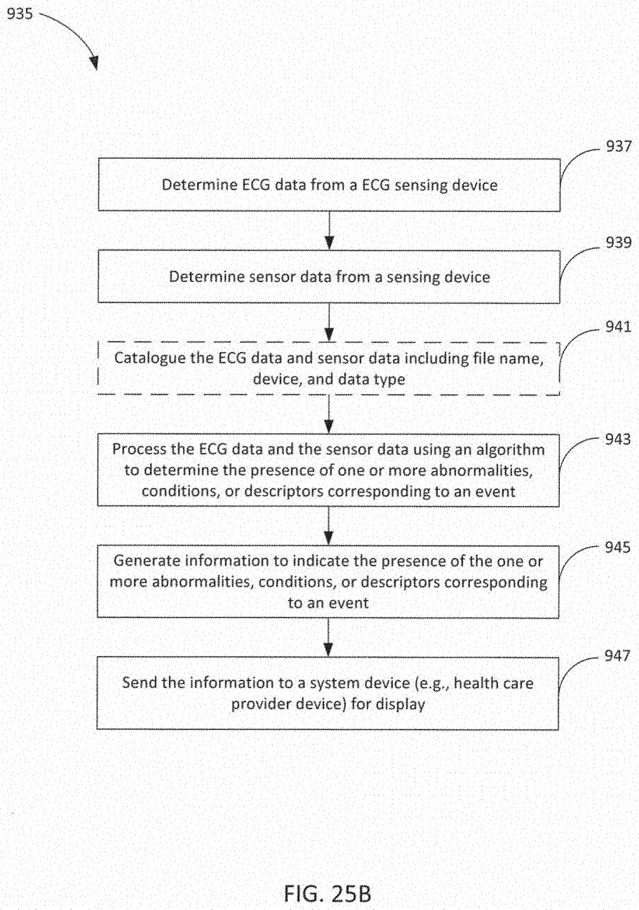

Referring now to FIGS. 25A-B, an exemplary system and process for multi-device ECG processing is illustrated. Referring now to FIG. 25A, ECG processing system 920 may include server 922, drive 924, system device 928, sensing device 930, and sensing device 932. Server 922 may be the same or similar to server 15 described above with respect to FIG. 2 and may run an ECG platform (e.g., ECG platform 37 described above with respect to FIG. 3A). Drive 924 may be the same or similar to drive 16 described above with respect to FIG. 2. System device 928 may be the same or similar to system device 14 described above with respect to FIG. 2. Sensing device 930 and/or sensing device 932 may be similar to sensing device 13 described above with respect to FIG. 2. Drive 924 may be incorporated into server 922 or may be separate and distinct from server 922 and/or may communicate with server 922 over any well-known wireless or wired connection. System device 928 may be in communication with server 922, sensing device 930 and/or sensing device 932 via any well-known wireless or wired connection. Further, sensing device 930 and/or sensing device 932 may be in communication with server 922 and/or system device 928 via any well-known wireless or wired connection.

Sensing device 930 and sensing device 932 may any type of device for sensing electrical activity of the heart, generating ECG data (e.g., ECG signals), and/or generating any other biometric or physiological data (e.g., heart rate, temperature, motion, oxygen levels (SpO2), respiratory rate, humidity, blood pressure, etc.). Sensing device 930 and sensing device 932 may be the same or different devices. For example, sensing device 930 may be a smart watch worn by user 925 and sensing device 932 may be an implantable ECG recording device (e.g., ILR). While only two sensing devices are illustrated in FIG. 25A, it is understood that processing system 920 may include more than two devices. Sensing devices may include other wearable devices and/or implantable devices.

Sensing device 930 and sensing device 932 may generate sensed data (e.g., ECG data and/or other biometric or physiological data) and may send such data to server 922. Sensing device 930 and sensing device 932 may send the data directly to server 922 or may send the data to server 922 via a computing device such as system device 928. Upon receiving the sensed data, server and/or drive 924 may analyze the data using one or more approaches or techniques described herein (e.g., process the sensed data to determine an anomaly, abnormality or condition). System device 928 may be used to analyze and otherwise oversee processing and analyzing the sensed data on server 922.

As shown in FIG. 25A, drive 924, which may be incorporated into server 922, may maintain databases such as database 926 to keep track of the different types of sensed data received from the various sensing devices (e.g., sensing device 930 and sensing device 932). For example, database 926 may assign a name (e.g., file name) to each of the received data and may associate the file name with the user or user account (e.g., patient no.) and may even identify the device that provided and/or generated the data as well as the type of data (e.g., heart rate (HR), SpO2, ECG, etc.). It is understood that the sensed data generated by the sensed devices and received by the server may be data other than ECG data, such as heart rate, respiratory rate, and other non-ECG data.

Referring now to FIG. 25B, a process for analyzing ECG and other data generated by a multi-device system for determining conditions, abnormalities, and/or descriptors is illustrated. To initiate multi-device process 935 (e.g., on an ECG platform), at step 937, ECG data from a sensing device is obtained and/or determined over a given time period (e.g., at a given sampling rate). At step 939, sensor data from a different sensing device (e.g., a smart watch or any other sensing device) is obtained and/or determined over a given time period. The sensor data may be any type of well-known physiological or biometric data (e.g., heart rate, SpO2, respiratory rate, etc.). In one example, the sensor data is generated by a photoplethysmogram (PPG) sensor. The time period for the sensor data and the ECG data may be the same or may overlap, even if the sampling rates are different.

At optional step 941, the ECG data and the sensor data may be catalogued or otherwise saved in an organized fashion (e.g., in a database) such that the ECG data and sensor data may be associated with the device from which it originated, the type of data, a file number, and/or any other information relevant to the ECG and/or sensor data. At step 943, the ECG data and sensor data may be processed using an algorithm to determine the presence of one or more abnormalities, conditions and/or descriptors corresponding to an event (e.g., cardiac event, ECG event, and/or any other type of physiological event). For example, techniques and/or algorithms similar to those described above (e.g., the techniques and/or algorithms described above with respect to FIG. 4) may be employed to analyze and/or process the sensor data and/or ECG data. It is understood that the various algorithms, neural networks, and models described above (e.g., the delineator and classifier) may be trained and/or otherwise designed to process both ECG data and other sensor data.

At step 945, information indicative of the presence of the one or more abnormalities, conditions, or descriptors corresponding to the event may be generated. For example, such information may be used to generate a display on a system device and/or generate a report regarding the one or more abnormalities, conditions, or descriptors. At step 947, the information generated at step 945 may be communicated to a system device for display. For example, the information may be sent or otherwise accessed by a health care provider device for display on the healthcare provider device.

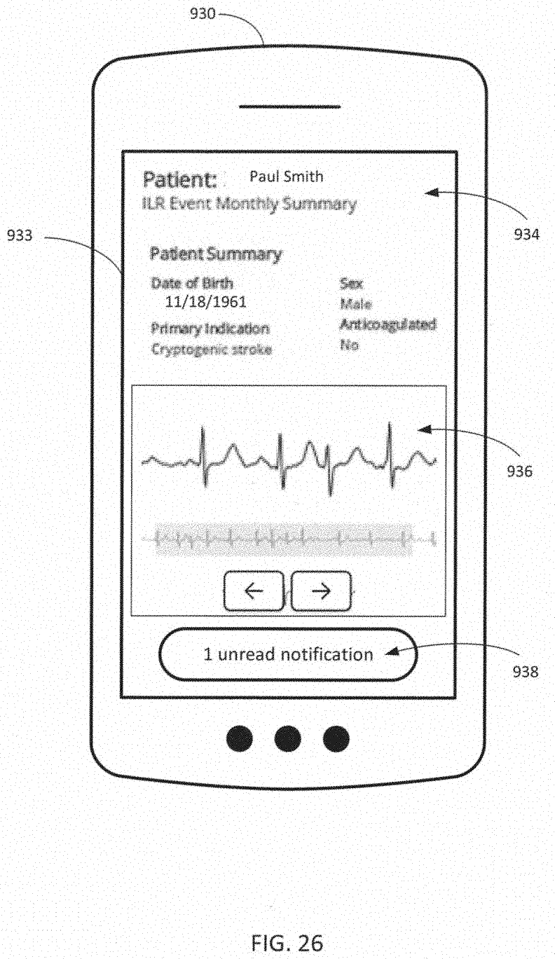

Referring now to FIG. 26, a mobile device presenting a mobile interface is illustrated. Mobile device 930 may be any type of computing device having a processor and a display and in communicate with a server, such as server 922, running an ECG platform (e.g., ECG platform 37 described above with respect to FIG. 3B). Mobile device 930 may have the same components or similar components to those described above with respect to FIG. 3A. For example, mobile device 930 may run an application (e.g., a local application) and may present mobile interface 933 on mobile device 930. Mobile interface 933 may include, for example, patient information 934, ECG information 936, and/or notification information 938.

The server running the ECG platform may communicate all or a portion of mobile interface 933 to mobile device 930. For example, mobile device 930 may communicate patient information 934, ECG information 936, and/or notification information 938 to mobile device 930, which may be presented by the application running on mobile device 930. Alternatively, and/or additionally, certain information presented on mobile interface 933 may be saved locally on mobile device 930. Patient information 934 may include information about the patient (e.g., date of birth, sex, indication, etc.). ECG information 936 may include ECG representation 936 which may be a representation of the ECG signal, such as portion of the signal at a detected ECG event.

ECG information 936 may optionally include information about a detected anomaly, descriptor and/or condition. Notification information 938 may include a notice that the user has a notification or message (e.g., from a health care provider and/or from the ECG platform running on the server). In one example, the notification may be a diagnosis or detected abnormality, condition, and/or anomaly determined by the ECG platform and/or the healthcare provider. Alternatively, or additionally, a notification may include a treatment recommendation Information displayed and provided by the ECG platform may have to be reviewed and/or released by a healthcare professional. Alternatively, the ECG platform may permit the mobile device to display such information once it has been reviewed and/or released by the healthcare professional. It is understood that different data and/or information than that illustrated in FIG. 26 may be presented by mobile interface 933.

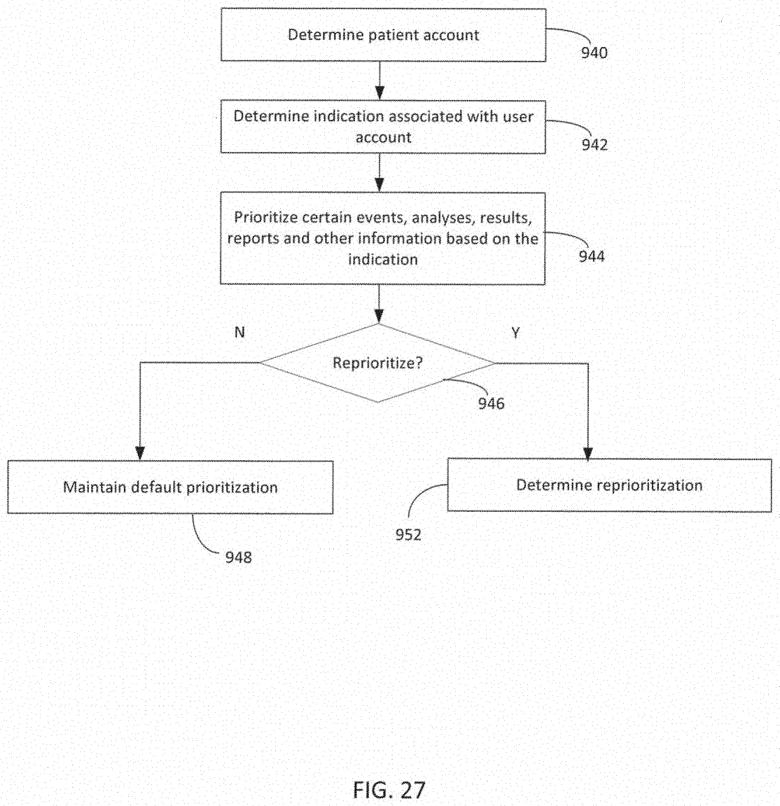

Referring now to FIG. 27, an exemplary process for prioritizing certain information for review by the healthcare provider is illustrated. As there may be many different types of analyses performed on various sensor data, and many different types of results, data and information generated or determined based on the sensed data, it may be useful to prioritize certain results, data, and/or information over others based on known information about the patient, such as an indication relevant to a particular patient. In this manner, the most important data, results, and information for the relevant indication may be presented to the healthcare provider before other less relevant data, results, and information. Some or all of the blocks of the process in FIG. 27 may be performed in a distributed manner across any number of devices (e.g., computing devices and/or servers). Some or all of the operations of the process in FIG. 27 may be optional and may be performed in a different order.

To initiate the process illustrated in FIG. 27 (e.g., on an ECG platform), step 940 may be executed to determine a patient account. For example, a patient name or identification may be used to identify a user account relevant to a specific patient. At step 942, an indication relevant to the patient account may be identified. For example, it may be determined that a particular patient has had a stroke or a heart attack. The patient account may include medical history about that patient and/or medical history about the family of the patient. The indication may be determined from the medical history or otherwise noted in the patient account.

At step 944, the system (e.g., ECG platform) may priority certain events, analyses, results, data, or other information determined by the system based on the indication identified at step 942. For example, results, data and/or other information determined by the system by analyzing sensed data (e.g., ECG data) may be prioritized for review by a healthcare professional. The prioritized data, results, and information may be known by the system to be associated or relevant to the indication. The system may include default settings making such associations between the data, results, identified abnormalities, conditions and/or events and/or information and certain indications.

At decision 946, the system may determine if the events, analyses, data, results, and/or information should be reprioritized. For example, the system may include a reprioritize button on a user interface presenting the events, analyses, data, results and/or information and the healthcare provider may engage the button to indicate that the presentation of the foregoing should be reprioritize or otherwise modified. If the data, results, and/or information should not be reprioritized (e.g., the healthcare provider did not engage the button), then at step 948, the default prioritization should be maintained. Alternatively, input from a user indicating that the data, results, and/or information associated with the indication should be reprioritized (e.g., the button was engaged), then at step 952, the data, results, and/or information prioritized for the indication should be reprioritized. For example, the healthcare provider may manually reprioritize such data, results, and/or information. Prioritization is described further below with respect to FIG. 31A.

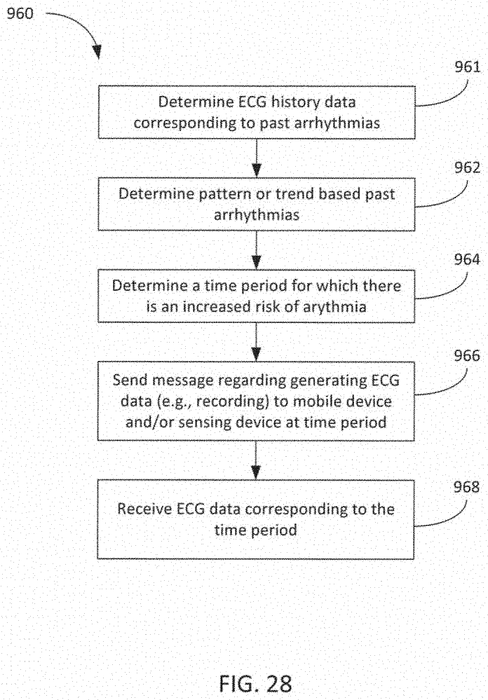

Referring now to FIG. 28, a process for determining a time period for recording ECG data likely to include an arrhythmia event is illustrated. It may be ideal to record ECG data when one or more events occur. However, it may be difficult to predict when such events will occur. Process 960 is an exemplary process for determining a time period for which there is an increased likelihood of an arrhythmia occurring and requesting ECG data corresponding to the time period. Some or all of the blocks of the process in FIG. 28 may be performed in a distributed manner across any number of devices (e.g., computing devices and/or servers). Some or all of the operations of the process in FIG. 28 may be optional and may be performed in a different order.

To initiate the process set forth in FIG. 28 (e.g., on an ECG platform), at step 961 a history of ECG data corresponding to past arrhythmias may be determined. For example, previous events corresponding to arrhythmias may be identified. At step 962, ECG data corresponding to previous events corresponding to arrhythmias may be processed or analyzed to determine a pattern or trend corresponding to the arrhythmias. For example, one or more trained models may be used to detect such patterns and/or trends. At step 964, the patterns and/or trends may be used to determine a time period for which there is an increased risk and/or likelihood of an arrhythmia occurring. The time period may correspond to a time of day, such as between 9:00 am and 9:30 am, for example.

At step 966, a message may be sent to a mobile device and/or to a sensing device to cause the sensing device to generate or obtain ECG data and/or other data relevant to the arrhythmia at the time period. For example, the message may be sent to a mobile device and the mobile device may request such data from the sensing device. Alternatively, the request may be sent directly to the sensing device. In yet another example, a user may need to manually cause the sensing device to record ECG data and the message may instruct the user to start recording the ECG at a certain time and/or for a certain duration. At step 968, the system may receive ECG data and/or other data relevant to the arrhythmia and corresponding to the time period. In this manner, the system and/or mobile device may trigger ECG recordings at times when the patient is likely to experience arrhythmias.

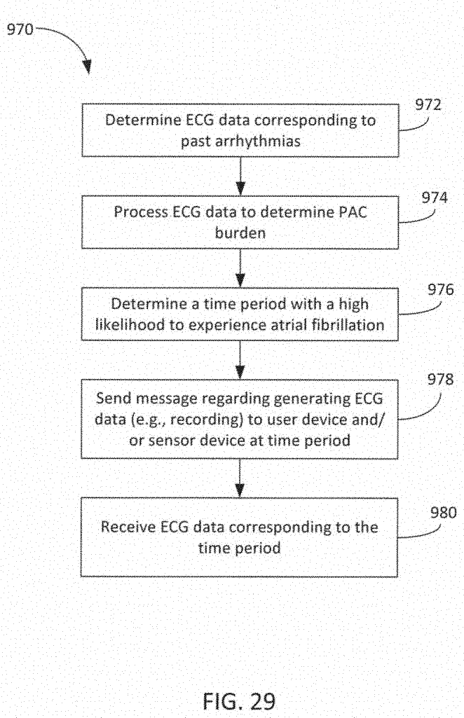

Referring now to FIG. 29, a process for determining a time period (e.g., interval) for recording ECG data likely to include an atrial fibrillation event based on a PAC burden is illustrated. As explained above, it may be difficult to predict when such arrhythmias will occur. Process 970 is an exemplary process for determining an interval for which there is an increased likelihood of an arrhythmia, and specifically atrial fibrillation occurring and requesting ECG data corresponding to the time period. Some or all of the blocks of the process in FIG. 29 may be performed in a distributed manner across any number of devices (e.g., computing devices and/or servers). Some or all of the operations of the process in FIG. 29 may be optional and may be performed in a different order.

To initiate the process set forth in FIG. 29 (e.g., on an ECG platform), at step 972 a history of ECG data corresponding to past arrhythmias may be determined. For example, previous events corresponding to arrhythmias may be identified. At step 974, the previous events corresponding to arrhythmias may be processed or analyzed to determine the total number of premature atrial contractions (PAC) over the total beats in a certain amount of time (i.e., the PAC burden). For example, the techniques described herein may be used to determine PACs in the ECG data and ultimately PAC burden. At step 976 a time period with a high likelihood to experience atrial fibrillation may be determined based on the PAC burden. For example, the techniques described herein may be used generate inferences regarding a likelihood of atrial fibrillation based on the PAC burden.

At step 978, a message may be sent to a mobile device and/or to a sensing device to cause the sensing device to generate or obtain ECG data and/or other data relevant during the time period. For example, the message may be sent to a mobile device and the mobile device may request such data from the sensing device. Alternatively, the request may be sent directly to the sensing device. In yet another example, a user may need to cause the sensing device to record ECG data and the message may instruct the user to start recording the ECG at a certain time. At step 968, the system may receive ECG data and/or other data relevant to the arrhythmia and corresponding to the time period. In this manner, the system and/or mobile device may trigger ECG recordings at times when the patient is likely to experience atrial fibrillation.

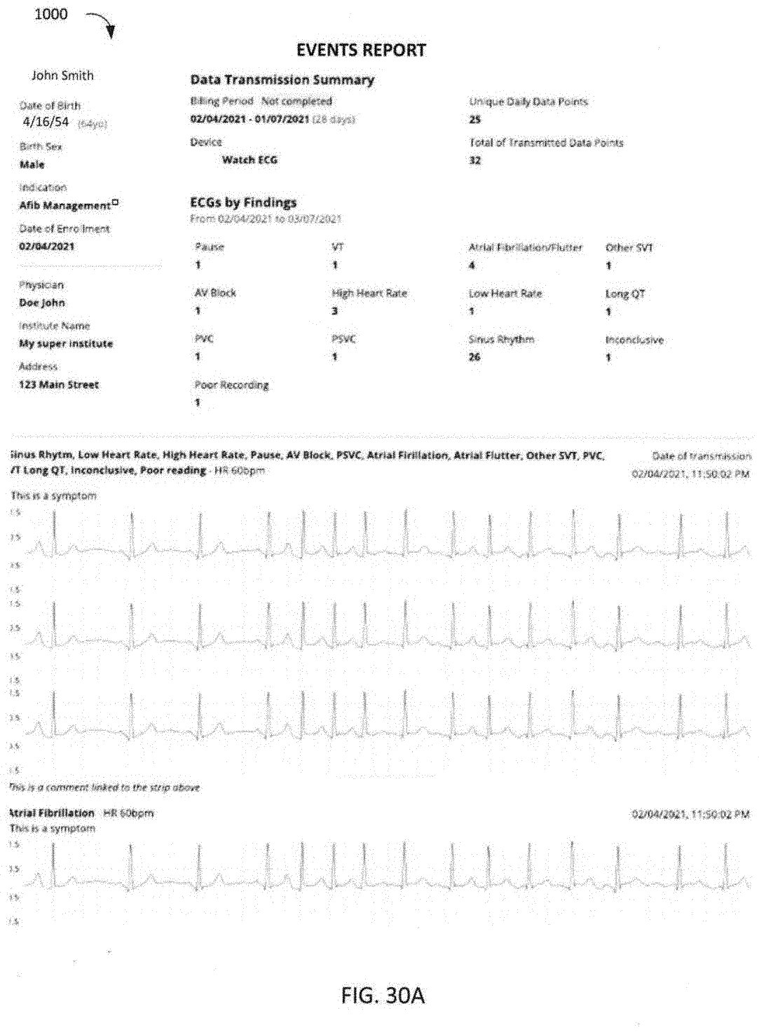

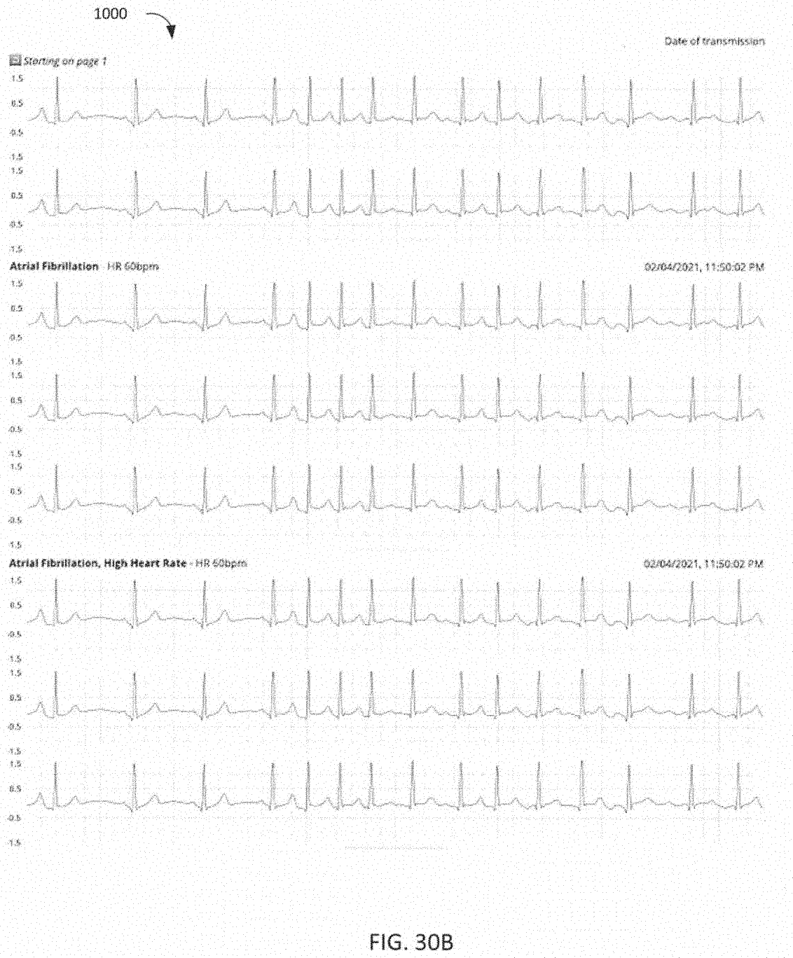

Referring now to FIGS. 30A-30B, an exemplary events report is illustrated. As shown in FIGS. 30A-30B, events report 1000 may include patient information (e.g., name, date of birth, indication, etc.), physician information (e.g., name, institution name, address, etc.), data transmission summary (e.g., device, transmitted data points, billing period, etc.), ECG findings summarizing abnormalities, descriptors, and/or conditions), and one or more ECG representations. For example, portions of ECG strips corresponding to the various abnormalities, descriptors, and/or conditions may be included in events report 1000.

Referring now to FIGS. 31A-31F, various user interfaces are illustrated for displaying patients, indications, classifications, and/or events. It is understood that the user interfaces illustrated in FIGS. 31A-31F may be displayed on any computing device described herein, such as system device 14 described above with respect to FIG. 2.

Referring now to FIG. 31A, patient registration interface 1004 is illustrated. As shown in FIG. 31A, patient registration interface 1004 may include entries for contact information (e.g., email, phone number, etc.), medical history (e.g., indication, medication, etc.) and may permit a healthcare provider to manually prioritize certain criteria (e.g., conditions, descriptors, abnormalities, other information).

Referring now to FIG. 31B, patient list interface 1006 is illustrated. As shown in FIG. 31B, patient list interface 1006 may include a list of patients (e.g., a list of patient's associated with a doctor and/or institution). Patient list interface 1006 may include the patient's name, a date of birth of the patient, an indication associated with the patient, enrollment data, an account status, and/or any other relevant information.

Referring now to FIG. 31C, registration interface 1010 is illustrated. As shown in FIG. 31D, registration interface 1010 may include entries for the patient's name, sex, date of birth, email, phone number, and/or medical history. For example, the medical history entries may include an entry for an indication corresponding to the patient and/or one or more medications taken by the patient.

Referring now to FIG. 31D, registration interface 1012, which may be the same as registration interface 1010, is illustrated. As shown in FIG. 31E, under the “medical history” section of registration interface 1012 there may be indication menu 1014 which may include indications that may be selected. For example, indications may be include Post Atrial Fibrillation ablation, Palpitations, AFib management, and/or none. It is understood that any other indication may be included in indication menu 1014.

Referring now to FIG. 31E, event interface 1018 is illustrated. As shown in FIG. 31C event interface may be accessed from an event list (e.g., by selecting a patient's name). Event interface FIG. 31C may include a patient's name, a classification for the event (e.g., atrial fibrillation), portions of ECG strips corresponding to the event, symptoms, heart rate, and/or any other relevant information. Event interface 1018 may further include a “reclassify” button for reclassifying the event. It is understood that the classification of the event may be determined by the ECG platform and/or may be determined by a sensing device (e.g., sensing device 930 of FIG. 25A).

Referring now to FIG. 31F, event interface 1020, which may be the same as registration interface 1018, is illustrated. As shown in FIG. 31F, event interface may include reclassification menu 1022 next to a classification provided in event interface 1020. For example, reclassification menu 1022 may include several reclassification options such as, sinus rhythm, low heart rate, high heart rate, pause, AV block, PSVC, atrial fibrillation, and/or any other condition, abnormality, and/or descriptor that may classify an event. In this manner, a classification provided by a sensing device (e.g., sensing device 930 of FIG. 25A) may be reclassified by a healthcare provider on using the ECG platform.

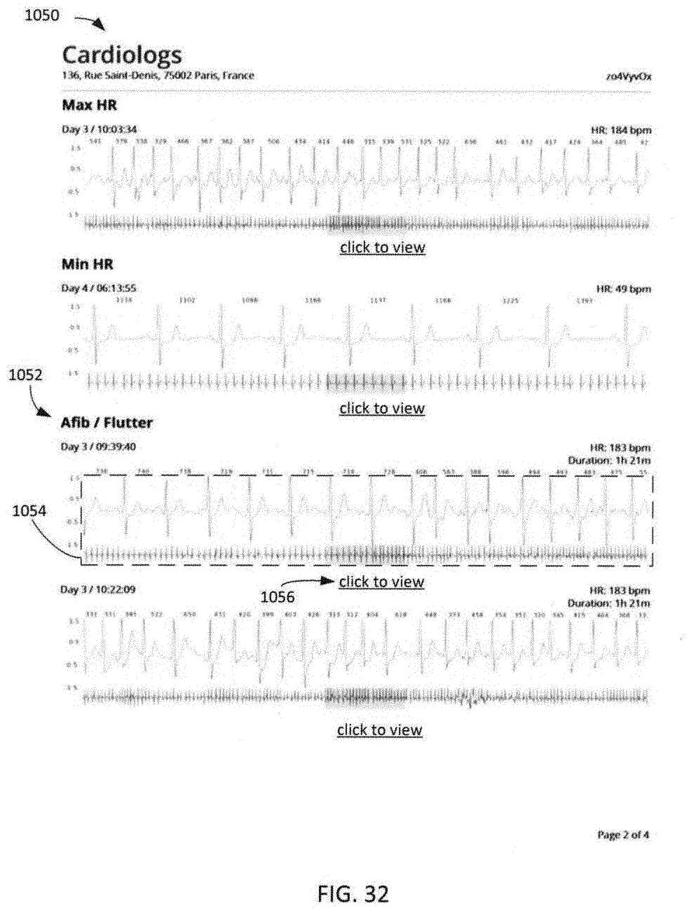

Referring now to FIG. 32, ECG report 1050 is illustrated which may be an exemplary portion of a more comprehensive ECG report such as the report described above with respect to FIGS. 15A-15D. As shown in FIG. 32, ECG report 1050 may include patient information such as the patient's name, primary indication, whether the patient has a pace maker, the patients date of birth, gender and/or a patient ID, and may also include other information such as the overseeing physician, the name of the institute, the date of the analysis and the like. It is understood that ECG report 1050 may be a digital rendering that may be presented on a computing device (e.g., laptop, desktop, tablet, mobile device, etc.) and/or may be a physical print out (e.g., on paper).

As shown in FIG. 32, ECG report 1050 may include various plots (e.g., plot 1052) corresponding to relevant ECG information and/or data. For example, ECG report 1050 may include ECG plots corresponding to maximum heart rate, minimum heart rate, atrial fibrillation, flutter, and/or any other type of ECG, cardiac, physiological and/or biological information. The plots (e.g., plot 1052) may be any type of plot such as an ECG strip, R-R plot, or heart rate density plot, for example. The plots may also indicate, identify or otherwise correspond to a medical condition, event and/or abnormality.

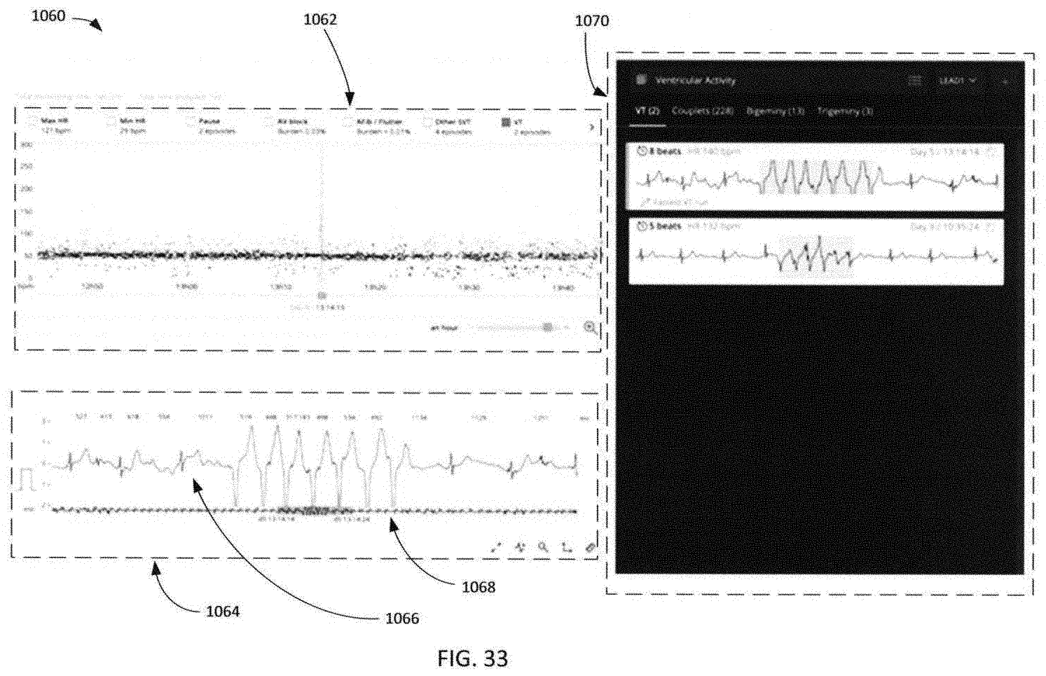

Plot 1052 and/or any other plot in ECG report 1050 may be interactive. For example, plot 1052 may include clickable portion 1054 and/or clickable link 1056, which each may be clicked or otherwise engaged by a user on a computing device. It is understood that clickable link 1056 may be text, an image, an icon, and/or the like. In one example, a physician and/or healthcare provider may receive a digital version of ECG report 1050 and may desire to view more of the signal and/or underlying data in more detail and thus may click clickable portion 1054 of a clickable ECG plot and/or clickable link 1056 using a computing device (e.g., using a touchscreen and/or mouse). Upon clicking clickable portion 1054 and/or clickable link 1056, the user may be redirected to ECG platform 37 and specifically to a viewer version of ECG application 29. For example, the user may be redirected to a viewer application (.eg., the viewer application and interface illustrated in FIG. 33). It is understood that ECG report 1050 may include one or more clickable link 1056 and/or clickable portion.

Referring now to FIG. 33, viewer interface 1060 of a viewer application is illustrated. The viewer application may permit a user, such as a user with limited viewing rights (e.g., a limited user), to view additional information corresponding to ECG data and/or other data identified in a report and/or otherwise provide limited access to an ECG platform. For example, the viewer application may generate viewer interface 1060 and may permit a limited user to view the full ECG signal and/or additional ECG data beyond that which was provided in the report. In this manner, the limited user may interact with viewer interface 1060 to view the ECG signals, ECG strips, ECG data, and/or other relevant information. It is further understood that a user with full access to the ECG platform may similarly access viewer application and viewer interface 1060.

As shown in FIG. 33, viewer interface 1060 may be similar to interactive display 101, described above with respect to FIG. 8. For example, viewer interface 1060 may include thee three distinct portions including first portion 1062, which may include a heart rate density plot, second portion 1064 which may include a focused ECG strip 1066 and expanded ECG strip 1068, and third portion 1070 which may include selectable ECG strips organized by identified conditions, events and/or abnormalities.

The heart rate density plot in first portion 1062 may be similar to plot 110 of FIG. 8 and/or may represent the entire signal or a portion thereof and may include selectable identifiers for visually identifying events, conditions and/or abnormalities identified in the ECG signal. Focused ECG strip 1066 may be an ECG strip of a particular timeframe in the heart rate density plot. Focused ECG strip 1066 may correspond to the location along a time axis of an interactive cursor of the heart rate density plot.

Expanded ECG strip 1068 may similarly correspond to a location of the interactive cursor on the on the heart rate density plot and may include an ECG strip having a length of time longer than focused ECG strip 1066 but including the timeframe of the focused ECG strip 1066. Expanded ECG strip 1068 may have a reduced height as compared to focused ECG strip 1066. It is understood that second portion 1064 and first portion 1066 may be linked such that moving the cursor on the heart rate density plot causes the portion of the ECG signal displayed in the focused ECG strip 1066 and the expanded ECG strip 1068 to change based on the location of the cursor on the time axis of the heart rate density plot.

The selectable ECG strips in third portion 1070 may be organized by identified conditions, events, and/or abnormalities. For example, the selectable ECG strips may be organized by ventricular tachycardia (VT), couplets, bigeminy, or trigeminy, for example. Each selectable ECG strip may be selected using the viewer application to view that portion of the ECG signal correspond to the selected ECG strip on first portion 1062 and the second portion 1064. Specifically, the cursor on the heart rate density plot may move to the portion of the heart rate density plot corresponding to the selected ECG strip. Further, focused ECG strip 1066 and expanded ECG strip 1068 will display the selected ECG strip and an expanded version of the selected ECG strip, respectively. In one example, the ECG strips in third portion 1070 may only be those strips included in the ECG report. Alternatively, all identified ECG strips by ECG system may be included in third portion 1070.

Viewer interface 1060 may display greater or fewer plots than that shown in FIG. 33, and/or may display other plots and/or other ECG, biological, physiological and/or any other relevant data. Furthermore, viewer interface 1060 may display comments and/or notes corresponding to the ECG data and/or strips and may optionally permit a limited user to make comments and/or notes. In yet another example, viewer interface 1060 may permit the limited user to provide feedback corresponding to the identified events, conditions and/or abnormalities. For example, the limited user may be able to identify or de-identify an ECG strip as associated with a given condition, event, and/or abnormality. Additionally, and/or alternatively, a limited user may modify and/or revise a report, add comments to a report, add conclusions to a report, and/or sign a report via viewer interface 1060.

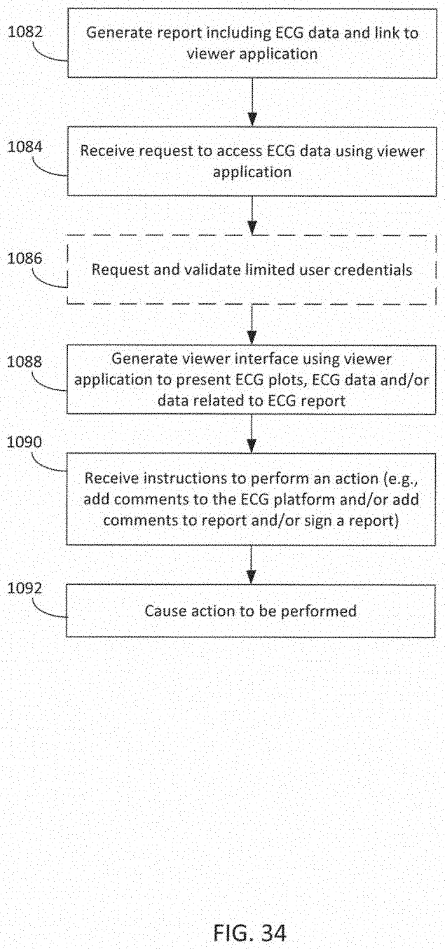

Referring now to FIG. 34, an exemplary process for redirecting a user from the report to the viewer application and viewer interface is depicted. To initiate the process, at block 1082 an ECG system may generate a report as described above with respect to step 68 of FIG. 4 and FIG. 33. At block 1084 an ECG may receive a request to access ECG data using a viewer application, which may be part of the ECG system (e.g., may be an application on the ECG platform). The request to access ECG data may be an automated request or message initiated by an individual viewing a report that has selected a selectable ECG strip and/or selectable link. For example, a healthcare provider may view a digital version of the ECG report on a computing device and may select a selectable ECG strip and/or link to be redirected to the viewer application.

At block 1086, the ECG system, in response to the request to access the viewer application, may request and validate user credentials. For example, the healthcare provider may be a registered limited user of the ECG system and may have a limited user profile with corresponding credentials (e.g., username and passcode). In response to receiving the request to access the viewer application, the ECG system may request the credentials from the limited user and may validate those credentials using the user profile.

At block 1088, the ECG system, via the viewer application, may generate a viewer interface to present ECG plots, ECG data, and/or other data related to the ECG report. For example, the ECG system may generate a viewer interface similar to viewer interface 1062, described above with respect to FIG. 33. At block 1090, the ECG system may receive instructions from user to perform an action (e.g., request to add comments to the ECG platform and/or add comments (e.g., conclusions) to and/or sign an ECG report). For example, a user may use the viewer application to move the user in the heart rate density plot to view various portions of the ECG signal, may select a selectable ECG strip for viewing, may request to add comments corresponding to an ECG strip, and/or may request to comment on and/or sign an ECG report. At block 1092, the ECG system may cause the action to be performed on the viewer application (e.g., sign report, add comments to report, add/or comments to ECG platform) based on the received instructions.

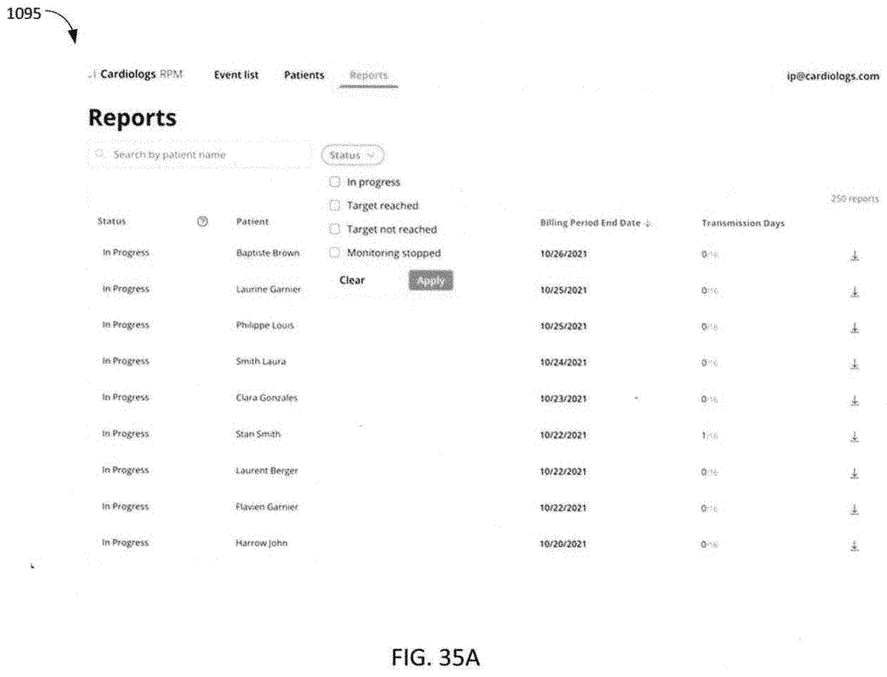

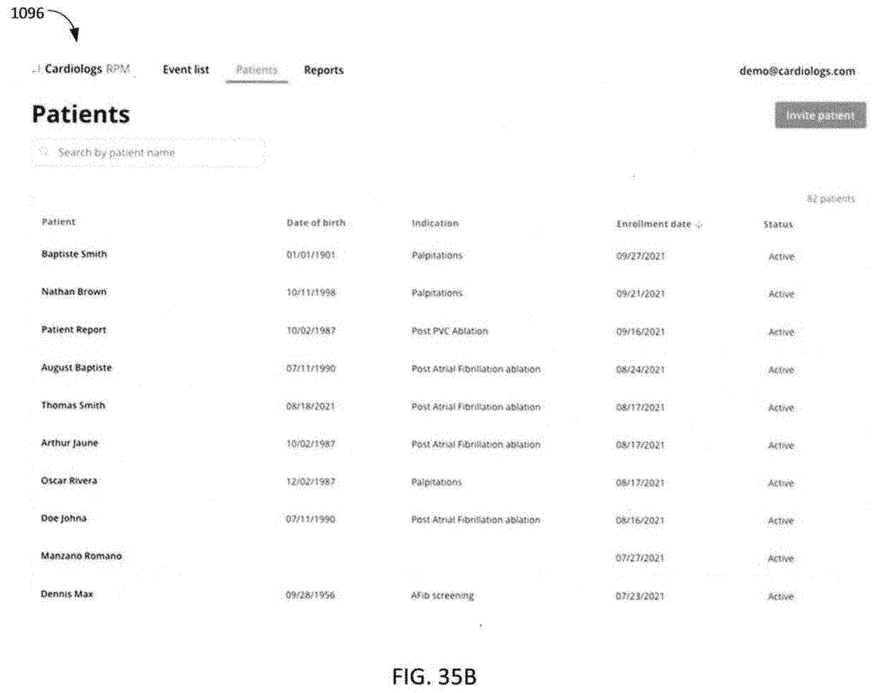

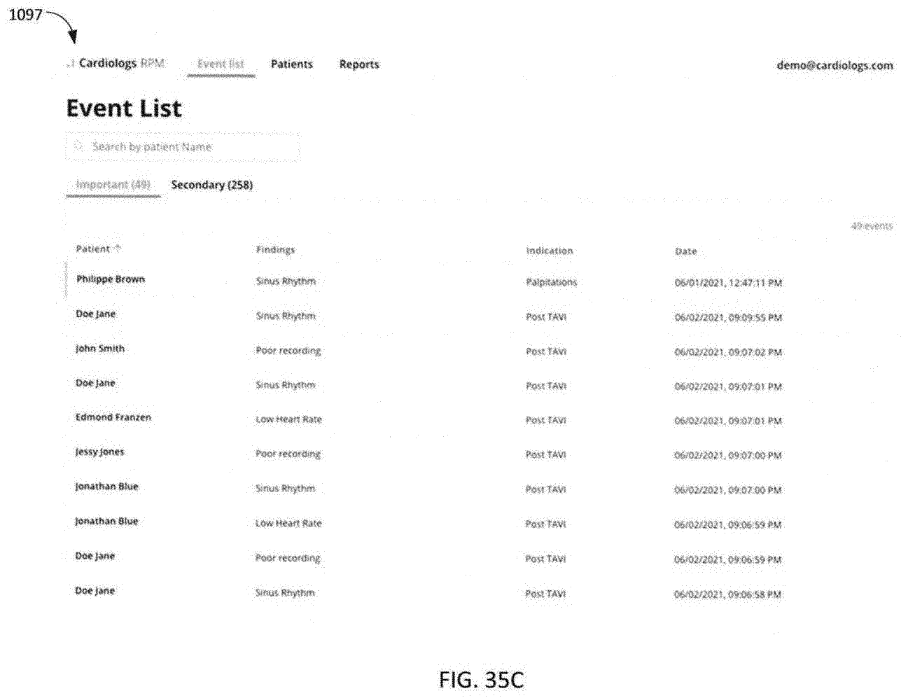

Referring now to FIGS. 35A-35C, report, patients and event list interfaces are illustrated. As shown in FIG. 35A, report interface 1095 may provide a status for reports in the ECG system. For example, report interface 1095 may include a column for the patient's name, the status of the report (e.g., in progress, target reached, target not reached, monitoring stopped), billing period end date, and/or transmission days. Further, report interface 1095 may include a search field (e.g., for the patient name) and a status filter (e.g., filter by in progress).

As shown in FIG. 35B, patient interface 1096 may include relevant information for patients in the ECG system. Patient interface 1096 may include a column for the patient's name, date of birth, indication, enrollment date, and/or status (e.g., active). Patient interface 1096 may include a search field (e.g., by patient name) and/or may be filtered.

As shown in FIG. 35C, event list interface 1097 may include relevant events for patients. For example, event list interface 1097 may include tabs for important and/or second secondary events and under each tab may include a column for patient name, findings (e.g., sinus rhythm, low heart rate, etc.) indication (e.g., palpitations) and/or date. Event list interface 1097 may include a search field (e.g., by patient name) and/or may be filtered.

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A computerized-method for analyzing electrocardiogram (ECG) data of a patient, the computerized method comprising:
obtaining, from a first device, a set of patient ECG data corresponding to a patient, the set of patient ECG data generated over a first plurality of time points as sampled by a sensing device;
obtaining, from a second device, a set of patient sensor data corresponding to the patient, the set of patient sensor data generated over a second plurality of time points, the second plurality of time points corresponding to the first plurality of time points;
processing at least a portion of the set of patient ECG data and at least a portion of the set of sensor data using an algorithm to determine a presence of one or more abnormalities, conditions, or descriptors corresponding to a cardiac event associated with the set of patient ECG data and the set of patient sensor data, the algorithm trained using a plurality of sets of ECG data different from the set of ECG data and a plurality of sets of sensor data different from the set of patient sensor data;
generating information, based on the processing, to indicate the presence of the one or more abnormalities, conditions, or descriptors corresponding to a cardiac event associated with the set of patient ECG data and set of patient sensor data; and
sending the information corresponding to the presence of the one or more abnormalities, conditions, or descriptors determined for the set of patient ECG data and the set of patient sensor data for display.

2. The computerized-method of claim 1, wherein the second device comprises a photoplethysmogram (PPG) sensor.

3. The computerized-method of claim 1, wherein the patient sensor data comprises one or more of heart rate, SpO2, respiratory rate data.

4. The computerized-method of claim 1, wherein the first device comprises an implantable loop recorder (ILR).

5. The computerized-method of claim 1, further comprising generating a database associating the ECG data with the first device and the patient sensor data with the second device.

6. The computerized-method of claim 1, further comprising obtaining, from the second device, a set of second sensor data corresponding to the patient and different than the set of patient sensor data.

7. The computerized-method of claim 6, wherein the set of second sensor data is generated over a third plurality of time points corresponding to the first plurality of time points.

8. The computerized-method of claim 6, further comprising processing at least a portion of the set of second sensor data using the algorithm, wherein the algorithm is further trained using a plurality of sets of second sensor data different from the set of second sensor data.

9. A computerized-method for analyzing electrocardiogram (ECG) data of a patient, the computerized-method comprising:
determining patient ECG data indicative of at least one cardiac event;
processing at least a portion of the patient ECG data using an algorithm to determine a presence of one or more descriptors corresponding to the at least one cardiac event associated with the patient ECG data, the algorithm trained using a plurality of sets of ECG data different from the patient ECG data;
determining a cardiac event and a descriptor corresponding to the cardiac event;
generating an event interface indicating the descriptor and comprising a graphical representation of the cardiac event; and
receiving input corresponding to the descriptor.

10. The computerized-method of claim 9, wherein the input reclassifies the cardiac event as a second descriptor.

11. The computerized-method of claim 10, further comprising generating an event interface indicating the second descriptor and comprising a graphical representation of the cardiac event.

12. The computerized-method of claim 10, wherein the second descriptor is used to train the algorithm.

13. The computerized-method of claim 9, wherein the event interface further comprises one or more of heart rate information or event duration information.

14. A computerized-method for analyzing electrocardiogram (ECG) data of a patient, the computerized-method comprising:
determining ECG history data, the ECG history data corresponding at least one arrhythmia event and sampled at a variety of time points;
processing ECG history data using an algorithm trained to determine a time point corresponding to a risk of an arrhythmia;
determining a first time period associated with the risk of an arrhythmia; and
sending a request for ECG data corresponding to the time period.

15. The computerized-method of claim 14, wherein the request for ECG data is sent to a user's mobile device.

16. The computerized-method of claim 14, wherein the request for ECG data is sent to a sensor device.

17. The computerized-method of claim 14, wherein the risk of an arrhythmia is a risk of atrial fibrillation.

18. The computerized-method of claim 14, wherein the algorithm is trained to determine a premature atrial contraction (PAC) burden.

19. A computerized-method for analyzing electrocardiogram (ECG) data of a patient, the computerized-method comprising:
determining the ECG data indicative of at least one ECG event;
processing ECG history data using an algorithm trained to determine at least one of a condition, descriptor or abnormality;
determining a plurality of results corresponding to the at least one the condition, descriptor or abnormality;
determining an indication associated with the patient;
determining a prioritized order of the plurality of results based on the indication; and
causing the prioritized order of the plurality of results to be presented on a computing device.

20. The computerized-method of claim 19, further comprising:
receiving a request to reprioritize the order of the plurality of results;
determining a second prioritized order of the plurality of results based on the request to reprioritize.

21. The computerized-method of claim 19, wherein the plurality of results comprises a first condition, and further comprising:
determining an association between the indication and a first condition; and
prioritizing the first condition based on the association.
